(19) United States
(12) Patent Application Publication
(10) Pub. No.: US 2012/0135980 A1
(43) Pub. Date: May 31, 2012

(54) GAMMA SECRETASE MODULATORS

(76) Inventors: Theodros Asberom, West Orange, NJ (US); Zhaoning Zhu, Plainsboro, NJ (US); John W. Clader, Milton, VT (US); Zhong-Yue Sun, Parlin, NJ (US); Mihirbaran Mandal, Scotch Plains, NJ (US); Gioconda Gallo, Summit, NJ (US); Xiaoxiang Liu, River Vale, NJ (US)

(21) Appl. No.: 13/140,992

(22) PCT Filed: Dec. 18, 2009
(86) PCT No.: PCT/US09/68685

§ 371 Date: Jan. 11, 2012

Related U.S. Application Data
(60) Provisional application No. 61/139,668, filed on Dec. 22, 2008.

(51) Int. Cl. A61K 31/553; A61P 25/00; A61K 31/55; A61K 31/551; C07D 498/04; C07D 513/04

(52) U.S. Cl. 514/211.09; 540/578; 540/568; 540/552; 514/214.02; 514/221

(57) ABSTRACT

This invention provides novel compounds that are modulators of gamma secretase. The compounds have the formula (Chemical formula should be inserted here as it appears on abstract in paper form). Also disclosed are methods of modulating gamma secretase activity and methods of treating Alzheimer's Disease using the compounds of formula (I).

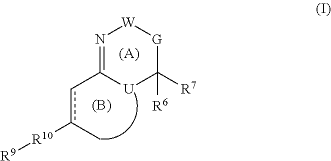

### REFERENCE TO RELATED APPLICATION

This Application claims the benefit of U.S. Provisional Application Ser. No. 61/139,668 filed Dec. 22, 2008.

### FIELD OF THE INVENTION

The present invention relates to certain heterocyclic compounds useful as gamma secretase modulators (including inhibitors, antagonists and the like), pharmaceutical compositions comprising the compounds, and methods of treating various diseases using the compounds and compositions. Examples of the diseases and conditions include, for example, Alzheimers disease, mild cognitive impairment (MCI), Downs Syndrome, Glaucoma, Cerebral amyloid angiopathy, stroke or dementia, Microgliosis and brain inflammation, and Olfactory function loss.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the β amyloid protein is critical for the seeding of amyloid formation: implications for the pathogenesis of Alzheimer's disease, Biochemistry, May 11, 1993, 32(18), p. 4693-4697) and constitute the main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.). Furthermore, it is known that mutations of APP and presenelin genes, which is are observed in familial Alzheimer's disease, increase production of Aβ40 and Aβ42 (for example, see Gouras G K, et al, Intraneuronal Aβ142 accumulation in human brain, American Journal of Pathology, January 2000, 156(1), p. 15-20. Also, see Scheuner D, et al, Nature Medicine, August 1996, 2(8), p. 864-870; and Forman M, et al, Differential effects of the Swedish mutant amyloid precursor protein on β-amyloid accumulation and secretion in neurons and normeuronal cells, Journal of Biological Chemistry, Dec. 19, 1997, 272(51), p. 32247-32253.). Therefore, compounds which reduce production of Aβ40 and Aβ42 are expected to be agents for controlling progress of Alzheimer's disease or for preventing the disease.

These Aβs are produced when APP is cleaved by beta secretase and subsequently cleaved by gamma secretase. In consideration of this, creation of inhibitors of γ-secretase and β-secretase has been attempted for the purpose of reducing production of Aβs. Many of these known secretase inhibitors are peptides or peptidomimetics such as L-685,458. L-685,458, an aspartyl protease transition state mimic, is a potent inhibitor of γ-secretase activity, Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone AG, published May 4, 2006); WO 2004/110350 (Neurogenetics, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

This invention provides novel compounds that are gamma secretase modulators, of the formula:

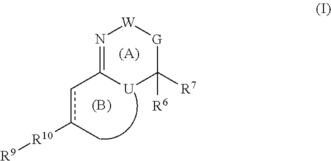

or a pharmaceutically acceptable salt, ester, or solvate thereof, wherein all substituents are defined below, and all substituents are independently selected.

This invention also provides compounds of formula (I).

This invention also includes the compounds of formula I in all its isolated forms.

This invention also provides compounds of formula (I) in pure and isolated form.

This invention also provides compounds of formula (I) selected from the group consisting of: the compounds of paragraphs (1) to (214).

This invention also provides compounds of formula (I) selected from the group consisting of: compounds 1A to 10A, 1B to 10B, 1C to 10C, 1D to 10D, 1E to 3E, 1F to 4F, 1G to 4G, the final Compounds of Examples 1, 2 and 4 to 9, and compounds 1H to 11H.

This invention also provides compounds of formula (I) selected from the group consisting of the final Compounds of Examples 1, 2 and 4 to 9, and compounds 1H to 11H.

This invention also provides pharmaceutical compositions comprising an effective amount of one or more (e.g., one) compounds of formula (I), or a pharmaceutically acceptable salt, ester or solvate thereof, and a pharmaceutically acceptable carrier.

This invention also provides pharmaceutical compositions comprising an effective amount of one or more (e.g., one) compounds of formula (I), or a pharmaceutically acceptable salt, ester or solvate thereof, and an effective amount of one or more (e.g., one) other pharmaceutically active ingredients (e.g., drugs), and a pharmaceutically acceptable carrier.

The compounds of Formula (I) can be useful as gamma secretase modulators and can be useful in the treatment and prevention of diseases such as, for example, central nervous system disorders such as Alzheimers disease and Downs Syndrome.

Thus, this invention also provides methods for: (1) method for modulating (including inhibiting, antagonizing and the like) gamma-secretase; (2) treating one or more neurodegenerative diseases; (3) inhibiting the deposition of amyloid protein (e.g., amyloid beta protein) in, on or around neurological tissue (e.g., the brain); (4) Alzheimer's disease; and (5) treating Downs syndrome; wherein each method comprises administering an effective amount of one or more (e.g., one) compounds of formula (I) to a patient in need of such treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula (I) and the administration of an effective amount of one or more (e.g., one) other pharmaceutical active ingredients (e.g., drugs).

This invention also provides methods for: (1) treating mild cognitive impairment; (2) treating glaucoma; (3) treating cerebral amyloid angiopathy; (4) treating stroke; (5) treating dementia; (6) treating microgliosis; (7) treating brain inflammation; and (8) treating olfactory function loss; wherein each method comprises administering an effective amount of one or more (e.g., one) compounds of formula (I) to a patient in need of such treatment.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described below), the combined quantities of the compound of formula (I) and the other pharmaceutically active ingredient being effective to treat the diseases or conditions mentioned in any of the above methods.

This invention also provides any of the above mentioned methods, pharmaceutical compositions or kit wherein the compound of formula (I) is selected from the group consisting of: the compounds of paragraphs (1) to (214).

This invention also provides any of the above mentioned methods, pharmaceutical compositions or kit wherein the compound of formula (I) is selected from the group consisting of: 1A to 10A, 1B to 10B, 1C to 10C, 1D to 10D, 1E to 3E, 1F to 4F, 1G to 4G, the final Compounds of Examples 1, 2 and 4 to 9, and compounds 1H to 11H.

This invention also provides any of the above mentioned methods, pharmaceutical compositions or kit wherein the compound of formula (I) is selected from the group consisting of: the final Compounds of Examples 1, 2 and 4 to 9, and compounds 1H to 11H.

### DETAILED DESCRIPTION OF THE INVENTION

This invention provides compounds, useful as gamma secretase modulators, of formula (I):

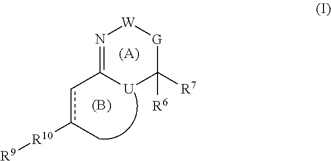

or a pharmaceutically acceptable salt, ester, or solvate thereof, wherein:

G, U, W, R⁶, R⁷, R⁹, and R¹⁰, are independently selected;

letters (A) and (B) in formula (I) are reference letters to identify the rings present in formula (I);

G is selected from the group consisting of —C(R³)(R⁴)—, —C(O)— and —N(R¹³)—, with the proviso that:
when W is —O— or —S— then G is not —N(R¹³)— or —C(O)—; and
when W is —SO— or —S(O)₂— then G is not —C(O)—;

U is CR⁵ or N;

W is selected from the group consisting of: —O—, —C(O)—, —S—, —S(O)—, —S(O₂)—, and —C(R¹¹)(R¹²)—;

the dotted line in Ring (B) represents an optional bond;

Ring (B) is a 5 to 8 membered ring (including the atoms common to Ring (A)), and: (1) when U is CR⁵ said Ring (B) optionally comprises 1 to 2 heteroatoms independently selected from the group consisting of O, NR² and S, and (2) when U is N said Ring (B) optionally comprises 1 to 2 additional heteroatoms independently selected from the group consisting of O, NR² and S; and said Ring (B) is optionally substituted with 1 to 5 independently selected R²¹ groups;

Each R² is independently selected from the group consisting of: H, —OH, —O-alkyl (i.e., alkoxy), —O-(halo substituted alky) (such as, for example, —O-fluoroalkyl), —NH(R^{4A}), —N(R^{4A})₂ (wherein each R^{4A} is independently selected), —NH₂, —S(O)R^{4A}, —S(O)(OR^{4A}), —S(O)₂R^{4A}, —S(O)₂(OR^{4A}), —S(O)NHR^{4A}, —S(O)N(R^{4A})₂, —S(O)NH₂, —S(O)₂NHR^{4A}, —S(O)₂N(R^{4A})₂, —S(O)₂NH₂, —CN, —C(O)₂R^{4A}, —C(O)NHR⁴, —C(O)N(R^{4A})₂, —C(O)NH₂, —C(O)R^{4A}, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

each R³ and R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-; wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- group is optionally substituted with 1-5 independently selected R²¹ groups;

each R^{4A} is independently selected from the group consisting of: unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

R⁵ is selected from the group consisting of: H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-; and wherein each of said R⁵ alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- groups are optionally substituted with 1-5 independently selected R²¹ substituents;

R⁶ and R⁷ are each independently selected from the group consisting of: H, —C(O)R¹⁵, —C(O)OR¹⁵, —C(O)N(R¹⁵)(R¹⁶), —C(═NOR¹⁵)R¹⁶, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, benzofusedcycloalkyl (i.e., fused benzocycloalkyl), fused benzoheterocycloalkyl, fused heteroarylcycloalkyl, fused heteroarylheterocycloalkyl; and wherein each of said R⁶ and R⁷ alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl-, benzofusedcycloalkyl, fused benzoheterocycloalkyl, fused heteroarylcycloalkyl, and fused heteroarylheterocycloalkyl group is optionally substituted with 1-5 independently selected R²¹ substituents; or

R⁶ and R⁷, taken together with the carbon atom to which they are bound, form a spirocyclic carbocyclic moiety or a spirocyclic heterocyclic moiety, and:
(a) optionally, said spirocyclic carbocyclic moiety is substituted with 1-4 independently selected R²¹ substituents,
(b) optionally, said spirocyclic heterocyclic moiety is substituted with 1-4 independently selected R²¹ substituents,
(c) optionally, said spirocyclic carbocyclic moiety is fused with an aryl, heteroaryl, cycloalkyl, or heterocycloalkyl ring to form a fused ring moiety, and optionally, each ring of said fused ring moiety is substituted with 1-4 independently selected R²¹ substituents;
(d) optionally, said spirocyclic heterocyclic moiety is fused with an aryl, heteroaryl, cycloalkyl, or heterocycloalkyl ring to form a fused ring moiety, and optionally, each ring of said fused ring moiety is substituted with 1-4 independently selected R²¹ substituents;

R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-; wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- group is optionally substituted with 1-3 independently selected R²¹ groups;

R⁹ is selected from the group consisting of: alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, and, optionally, each R⁹ group is substituted with 1-3 independently selected R²¹ groups;

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl-,

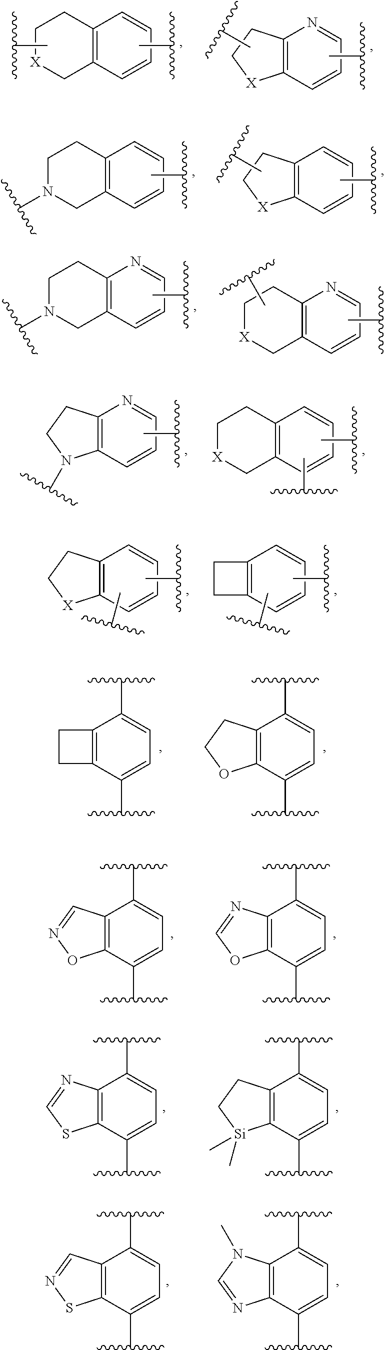

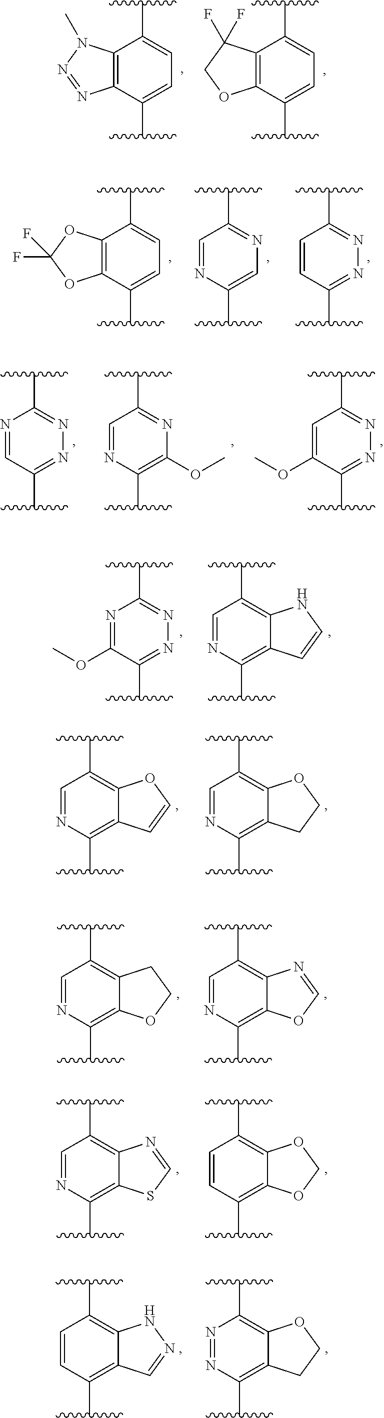

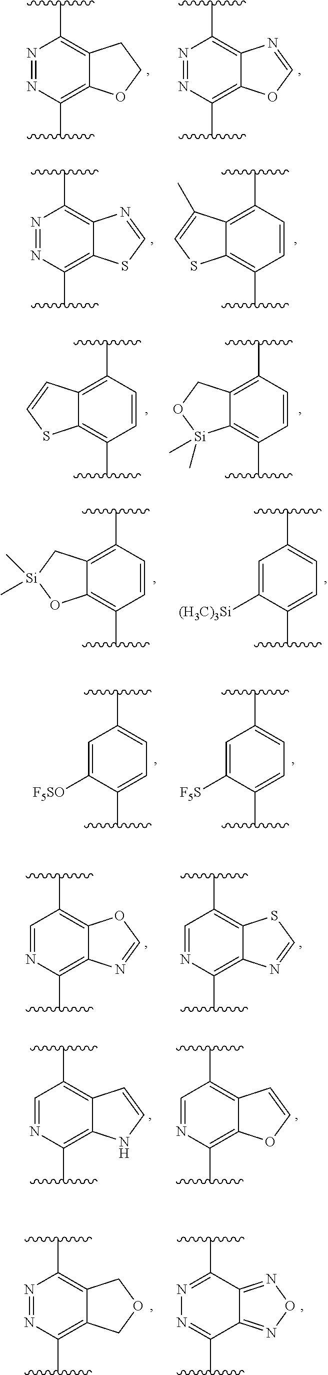

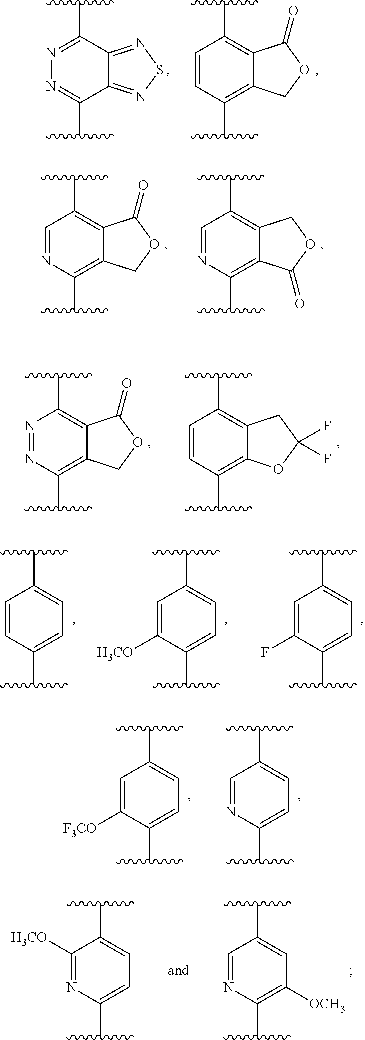

wherein X is selected from the group consisting of: O, N(R¹⁴) or S; and, optionally, each of said R¹⁰ groups are substituted with 1-3 independently selected R²¹ substitutents;

R¹¹ and R¹² are each independently selected from the group consisting of: H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-; and wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- group is optionally substituted with 1-5 independently selected R²¹;

R¹³ is independently selected from the group consisting of H, alkyl, arylalkyl, heteroarylalkyl-, cycloalkylalkyl-, heterocycloalkylalkyl-, arylcycloalkylalkyl-, heteroarylcycloalkylalkyl-, arylheterocycloalkylalkyl-, heteroaryiheterocycloalkylalkyl-, cycloalkyl, arylcycloalkyl-, heteroarylcycloalkyl-, heterocycloalkyl-, arylheterocycloalkyl-, heteroarylheterocycloalkyl-, alkenyl, arylalkenyl-, cycloalkenyl, arylcycloalkenyl-, heteroarylcycloalkenyl-, heterocycloalkenyl, arylheterocycloalkenyl-, heteroarylheterocycloalkenyl-, alkynyl, arylalkynyl-, aryl, cycloalkylaryl-, heterocycloalkylaryl-, heterocycloalkenylaryl-, heteroaryl, cycloalkylheteroaryl-, heterocycloalkylheteroaryl-, cycloalkenylaryl-, heterocycloalkenylaryl-, —OR¹⁵, —CN, —C(O)R⁸, —C(O)OR⁹, —S(O)R¹⁰, —S(O)₂R¹⁰, —C(O)N(R¹¹)(R¹²), —S(O)N(R¹¹)(R¹²), —S(O)₂N(R¹¹)(R¹²), —NO₂, —N═C(R⁸)₂ and —N(R⁸)₂; and wherein said R¹³ alkyl, arylalkyl-, heteroarylalkyl-, cycloalkylalkyl-, heterocycloalkylalkyl-, arylcycloalkylalkyl-, heteroarylcycloalkylalkyl-, arylheterocycloallylalkyl-, heteroarylheterocycloalkylalkyl-, cycloalkyl, arylcycloalkyl-, heteroarylcycloalkyl-, heterocycloalkyl, arylheterocycloalkyl-, heteroarylheterocycloalkyl-, alkenyl, arylalkenyl-, cycloalkenyl, arylcycloalkenyl-, heteroarylcycloalkenyl-, heterocycloalkenyl, arylheterocycloalkenyl-, heteroarylheterocycloalkenyl-, alkynyl, arylalkynyl-, aryl, cycloalkylaryl-, heterocycloalkylaryl-, heterocycloalkenylaryl-, heteroaryl, cycloalkylheteroaryl-, heterocycloalkylheteroaryl-, cycloalkenylaryl-, and heterocycloalkenylaryl- groups are optionally substituted with 1 to 5 groups independently selected from the group consisting of: alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R^{16A}, —N(R¹⁵)S(O)₂R^{16A}, —CH₂—N—N(R¹⁵)S(O)₂N(R¹⁶)(R¹⁷), —N(R¹⁵)_{S}(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, ═NOR¹⁵, —N₃, —NO₂ and —S(O)₂R^{15A};

R¹⁴ is selected from the group consisting of H, alkyl, alkenyl, alkynyl, cycloalkyl, cycloalkylalkyl-, cycloalkenyl, heterocyclyl, heterocyclenyl, heterocyclylalkyl-, heterocyclyalkenyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), and wherein each alkyl, alkenyl, alkynyl, cycloalkyl, cycloalkylalkyl-, cycloalkenyl, heterocyclyl, heterocyclenyl, heterocyclylalkyl-, heterocyclyalkenyl-, aryl, arylalkyl-, heteroaryl, heteroarylalkyl- group is optionally substituted with 1-5 independently selected R²¹ groups;

Each R^{15A} is independently selected from the group consisting of alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁹)₁₋₅-alkyl, (R¹⁸)₁₋₅-cycloalkyl, (R¹⁹)₁₋₅-cycloalkylalkyl-, (R¹⁹)₁₋₅-heterocyclyl, (R¹⁸)₁₋₅-heterocyclylalkyl-, (R¹⁸)₁₋₅-aryl, (R¹⁸)₁₋₅-arylalkyl-, (R¹⁸)₁₋₅-heteroaryl and (R¹⁸)₁₋₅-heteroarylalkyl-; and wherein each R¹⁸ in each group can be on any substitutable atom;

Each R^{16A} is independently selected from the group consisting of alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)₁₋₅-alkyl, (R¹⁸)₁₋₅-cycloalkyl, (R¹⁹)₁₋₅-cycloalkylalkyl-, (R¹⁹)₁₋₅-heterocyclyl, (R¹⁹)₁₋₅-heterocyclylalkyl-, (R¹⁸)₁₋₅-aryl, (R¹⁸)₁₋₅-arylalkyl-, (R¹⁸)₁₋₅-heteroaryl and (R¹⁸)₁₋₅-heteroarylalkyl-; and wherein each R¹⁸ in each group can be on any substitutable atom;

R¹⁵, R¹⁶ and R¹⁷ are independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)₁₋₅-alkyl, (R¹⁹)₁₋₅-cycloalkyl, (R¹⁹)₁₋₅-cycloalkylalkyl-, (R¹⁸)₁₋₅-heterocyclyl, (R¹⁸)₁₋₅-heterocyclylalkyl-, (R¹⁸)₁₋₅-aryl, (R¹⁸)₁₋₅-arylalkyl-, (R¹⁹)₁₋₅-heteroaryl and (R¹⁸)₁₋₅-heteroarylalkyl-; and wherein each R¹⁸ in each group can be on any substitutable atom;

Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, two R¹⁸ moieties on adjacent carbons can be taken together with the atoms to which they are bound to form:

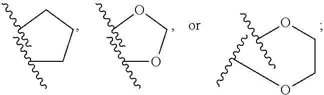

R¹⁹ is selected from the group consisting of: alkyl, cycloalkyl, aryl, arylalkyl and heteroarylalkyl;

R²⁰ is selected from the group consisting of: alkyl, cycloalkyl, aryl, halo substituted aryl, arylalkyl, heteroaryl and heteroarylalkyl;

Each R²¹ is independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SF₅, —OSF₅, —Si(R^{15A})₃ wherein each R^{15A} is independently selected, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, —N₃, —NO₂ and —S(O)₂R^{15A}; and, optionally, each of said alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl R²¹ groups are substituted with 1 to 5 independently selected R²² groups; and

Each R²² is independently selected from the group consisting of: alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R^{15A})₃ wherein each R^{15A} is independently selected, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)_{C}(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R^{15A} and —S(O)₂R^{15A}.

Those skilled in the art will appreciate that the moiety:

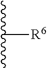

can have the stereochemistry

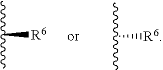

The moiety

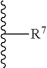

can have the stereochemistry

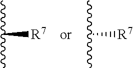

Thus, in one embodiment of this invention the R⁶ and R⁷ moieties can have the stereochemistry:

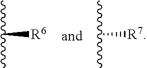

And in another embodiment of this invention the R⁶ and R⁷ moieties can have the stereochemistry:

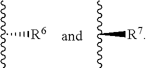

The R⁶ and R⁷ benzofusedcycloalkyl (i.e., fused benzocycloalkyl), fused benzoheterocycloalkyl, fused heteroarylcycloalkyl, and fused heteroarylheterocycloalkyl groups, can be optionally substituted with 1-5 independently selected R²¹ groups. In one example, the R²¹ groups are halo (e.g., F).

Examples of the fused ring R⁶ and R⁷ groups include, but are not limited to:

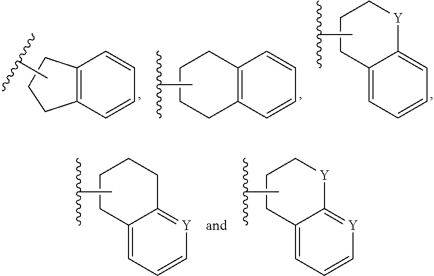

wherein each Y is independently selected from the group consisting of: —O—, —NR¹⁴— and —C(R²¹)_{q}— (wherein q is 0, 1 or 2 and each R²¹ is independently selected), and wherein R¹⁴ and R²¹ are as defined for formula (I). Examples of these fused ring R⁶ and R⁷ groups include, for example:

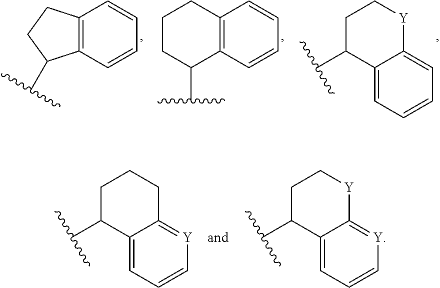

The compounds of this invention are useful for treating central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

Thus, for example, the compounds of this invention can be used to treat the following diseases or conditions: Alzheimers disease, mild cognitive impairment (MC1), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P Lamber, Proc. Natl. Acad. Sci. USA 95, 6448-53 (1998)), and Olfactory function loss (Getchell, et. al. Neurobiology of Aging, 663-673, 24, 2003).

In one embodiment R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl-,

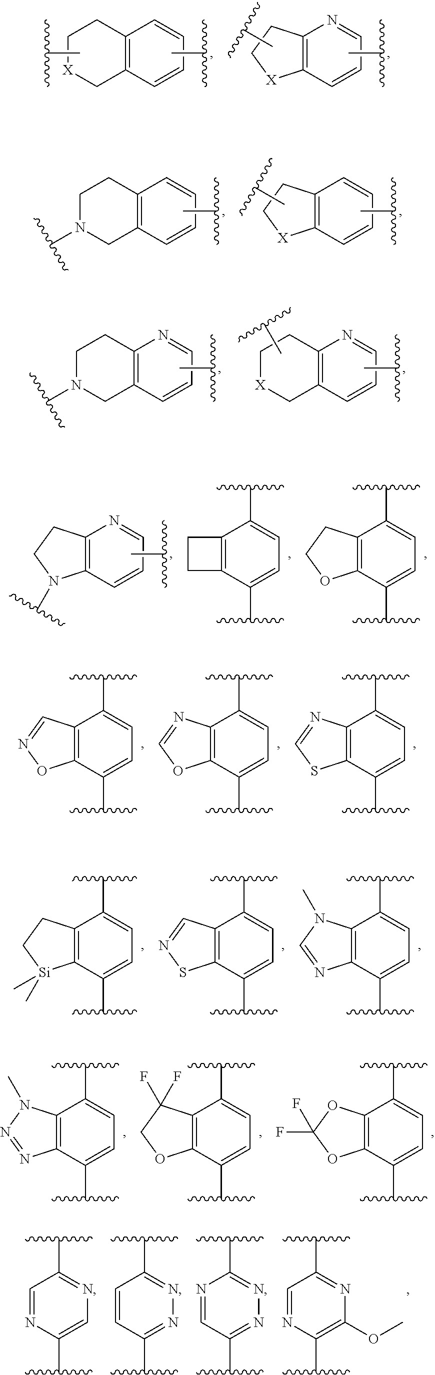

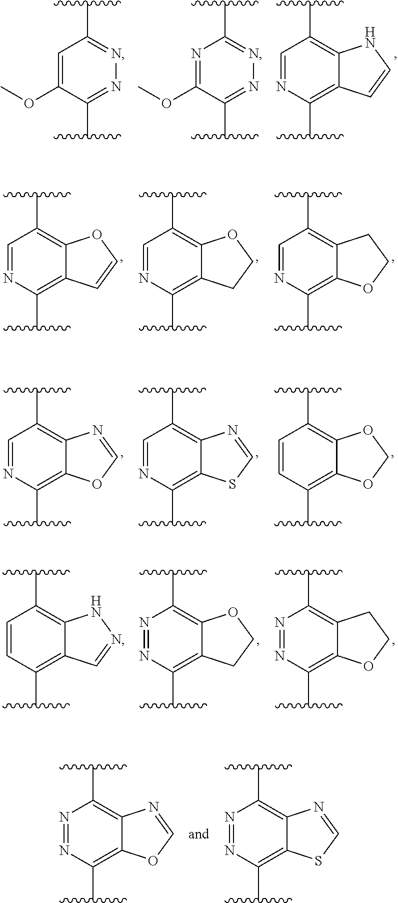

wherein X is selected from the group consisting of: O, N(R¹⁴) or S; and, optionally, each of said R¹⁰ groups are substituted with 1-3 independently selected R²¹ substitutents.

In one embodiment of this invention U is CR⁵.

In another embodiment of this invention U is N.

In one embodiment of this invention B is a five membered ring.

In another embodiment of this invention B is a five membered ring and the optional bond is present.

In another embodiment of this invention B is a five membered ring and the optional bond is absent.

In one embodiment of this invention B is a six membered ring.

In another embodiment of this invention B is a six membered ring and the optional bond is present.

In another embodiment of this invention B is a six membered ring and the optional bond is absent.

In one embodiment of this invention B is a seven membered ring.

In another embodiment of this invention B is a seven membered ring and the optional bond is present.

In another embodiment of this invention B is a seven membered ring and the optional bond is absent.

In one embodiment of this invention B is an eight membered ring.

In another embodiment of this invention B is an eight membered ring and the optional bond is present.

In another embodiment of this invention B is an eight membered ring and the optional bond is absent.

In one embodiment of this invention B is a five membered ring and U is CR⁵.

In another embodiment of this invention B is a five membered ring, U is CR⁵, and the optional bond is present.

In another embodiment of this invention B is a five membered ring, U is CR⁵, and the optional bond is absent.

In one embodiment of this invention B is a six membered ring, and U is CR⁵.

In another embodiment of this invention B is a six membered ring, U is CR⁵, and the optional bond is present.

In another embodiment of this invention B is a six membered ring, U is CR⁵, and the optional bond is absent.

In one embodiment of this invention B is a seven membered ring, and U is CR⁵.

In another embodiment of this invention B is a seven membered ring, U is CR⁵, and the optional bond is present.

In another embodiment of this invention B is a seven membered ring, U is CR⁵, and the optional bond is absent.

In one embodiment of this invention B is an eight membered ring, and U is CR⁵.

In another embodiment of this invention B is an eight membered ring, U is CR⁵, and the optional bond is present.

In another embodiment of this invention B is an eight membered ring, U is CR⁵, and the optional bond is absent.

In one embodiment of this invention B is a five membered ring and U is N.

In another embodiment of this invention B is a five membered ring, U is N, and the optional bond is present.

In another embodiment of this invention B is a five membered ring, U is N, and the optional bond is absent.

In one embodiment of this invention B is a six membered ring, and U is N.

In another embodiment of this invention B is a six membered ring, U is N, and the optional bond is present.

In another embodiment of this invention B is a six membered ring, U is N, and the optional bond is absent.

In one embodiment of this invention B is a seven membered ring, and U is N.

In another embodiment of this invention B is a seven membered ring, U is N, and the optional bond is present.

In another embodiment of this invention B is a seven membered ring, U is N, and the optional bond is absent.

In one embodiment of this invention B is an eight membered ring, and U is N.

In another embodiment of this invention B is an eight membered ring, U is N, and the optional bond is present.

In another embodiment of this invention B is an eight membered ring, U is N, and the optional bond is absent.

In one embodiment of this invention B is a five membered ring, U is CR⁵, and there are 1 or 2 heteroatoms present in ring B.

In another embodiment of this invention B is a five membered ring, U is CR⁵, there are 1 or 2 heteroatoms present in ring B, and the optional bond is present.

In another embodiment of this invention B is a five membered ring, U is CR⁵, there are 1 or 2 heteroatoms present in ring B, and the optional bond is absent.

In one embodiment of this invention B is a six membered ring, and U is CR⁵, and there are 1 or 2 heteroatoms present in ring B.

In another embodiment of this invention B is a six membered ring, U is CR⁵, there are 1 or 2 heteroatoms present in ring B, and the optional bond is present.

In another embodiment of this invention B is a six membered ring, U is CR⁵, there are 1 or 2 heteroatoms present in ring B, and the optional bond is absent.

In one embodiment of this invention B is a seven membered ring, U is CR⁵, and there are 1 or 2 heteroatoms present in ring B.

In another embodiment of this invention B is a seven membered ring, U is CR⁵, there are 1 or 2 heteroatoms present in ring B, and the optional bond is present.

In another embodiment of this invention B is a seven membered ring, U is CR⁵, there are 1 or 2 heteroatoms present in ring B, and the optional bond is absent.

In one embodiment of this invention B is an eight membered ring, U is CR⁵, and there are 1 or 2 heteroatoms present in ring B.

In another embodiment of this invention B is an eight membered ring, U is CR⁵, there are 1 or 2 heteroatoms present in ring B, and the optional bond is present.

In another embodiment of this invention B is an eight membered ring, U is CR⁵, there are 1 or 2 heteroatoms present in ring B, and the optional bond is absent.

In one embodiment of this invention B is a five membered ring, U is N, and there are 1 or 2 additional heteroatoms present in ring B.

In another embodiment of this invention B is a five membered ring, U is N, there are 1 or 2 additional heteroatoms present in ring B, and the optional bond is present.

In another embodiment of this invention B is a five membered ring, U is N, there are 1 or 2 additional heteroatoms present in ring B, and the optional bond is absent.

In one embodiment of this invention B is a six membered ring, U is N, and there are 1 or 2 additional heteroatoms present in ring B.

In another embodiment of this invention B is a six membered ring, U is N, and the optional bond is present.

In another embodiment of this invention B is a six membered ring, U is N, there are 1 or 2 additional heteroatoms present in ring B, and the optional bond is absent.

In one embodiment of this invention B is a seven membered ring, U is N, and there are 1 or 2 additional heteroatoms present in ring B.

In another embodiment of this invention B is a seven membered ring, U is N, there are 1 or 2 additional heteroatoms present in ring B, and the optional bond is present.

In another embodiment of this invention B is a seven membered ring, U is N, there are 1 or 2 additional heteroatoms present in ring B, and the optional bond is absent.

In another embodiment of this invention B is a seven membered ring, U is N, one 1 additional heteroatom is present in ring B, and the optional bond is absent.

In another embodiment of this invention B is a seven membered ring, U is N, one 1 additional heteroatom is present in ring B, said additional heteroatom is N (i.e., a —NH— moiety), and the optional bond is absent.

In one embodiment of this invention B is an eight membered ring, U is N, and there are 1 or 2 additional heteroatoms present in ring B

In another embodiment of this invention B is an eight membered ring, U is N, there are 1 or 2 additional heteroatoms present in ring B, and the optional bond is present.

In another embodiment of this invention B is an eight membered ring, U is N, there are 1 or 2 additional heteroatoms present in ring B, and the optional bond is absent.

The paragraphs below ((1) to (250)) are numbered for ease of reference.

(1) In one embodiment of this invention W is —O—.

(2) In another embodiment of this invention W is —O— and U is N.

(3) In another embodiment of this invention W is —O—, U is N, R³ is H, and R⁴ is H.

(4) In another embodiment of this invention W is —O—, U is N, and R³ and R⁴ are independently selected from the group consisting of: H and alkyl (e.g. methyl).

(5) In another embodiment of this invention W is —O— and U is CR⁵.

(6) In another embodiment of this invention W is —O—, U is CR⁵, and R⁵ is H.

(7) In another embodiment of this invention W is —O—, U is CR⁵, and R⁵ is alkyl (e.g. methyl).

(8) In another embodiment of this invention W is —O—, U is CR⁵, R³ is H, and R⁴ is H.

(9) In another embodiment of this invention W is —O—, U is CR⁵, R³ is H, R⁴ is H, and R⁵ is H.

(10) In another embodiment of this invention W is —O—, U is CR⁵, R³ is H, R⁴ is H, and R⁵ is alkyl (e.g. methyl).

(11) In another embodiment of this invention W is —O—, U is CR⁵, R³ and R⁴ are independently selected from the group consisting of: H and alkyl (e.g. methyl), and R⁵ is H

(12) In another embodiment of this invention W is —O—, U is CR⁵, R³ and R⁴ are independently selected from the group consisting of: H and alkyl (e.g. methyl), and R⁵ is alkyl (e.g. methyl).

(13) In one embodiment of this invention W is —S—.

(14) In another embodiment of this invention W is —S— and U is N.

(15) In another embodiment of this invention W is —S—, U is N, R³ is H, and R⁴ is H.

(16) In another embodiment of this invention W is —S—, U is N, and R³ and R⁴ are independently selected from the group consisting of: H and alkyl (e.g. methyl).

(17) In another embodiment of this invention W is —S— and U is CR⁵.

(18) In another embodiment of this invention W is —S—, U is CR⁵, and R⁵ is H.

(19) In another embodiment of this invention W is —S—, U is CR⁵, and R⁵ is alkyl (e.g. methyl).

(20) In another embodiment of this invention W is —S—, U is CR⁵, R³ is H, and R⁴ is H.

(21) In another embodiment of this invention W is —S—, U is CR⁵, R³ is H, R⁴ is H, and R⁵ is H.

(22) In another embodiment of this invention W is —S—, U is CR⁵, R³ is H, R⁴ is H, and R⁵ is alkyl (e.g. methyl).

(23) In another embodiment of this invention W is —S—, U is CR⁵, R³ and R⁴ are independently selected from the group consisting of: H and alkyl (e.g. methyl), and R⁵ is H.

(24) In another embodiment of this invention W is —S, U is CR⁵, R³ and R⁴ are independently selected from the group consisting of: H and alkyl (e.g. methyl), and R⁵ is alkyl (e.g. methyl).

(25) In one embodiment of this invention W is —C(O)—.

(26) In another embodiment of this invention W is —C(O)— and U is N.

(27) In another embodiment of this invention W is —C(O)—, U is N, and G is —C(R³)(R⁴).

(28) In another embodiment of this invention W is —C(O)—, U is N, G is —C(R³)(R⁴), R³ is H, and R⁴ is H.

(29) In another embodiment of this invention W is —C(O)—, U is N, G is —C(R³)(R⁴), and R³ and R⁴ are each independently selected from the group consisting of: H and alkyl (e.g. methyl).

(30) In another embodiment of this invention W is —C(O)— and U is CR⁵.

(31) In another embodiment of this invention W is —C(O)—, U is CR⁵, and R⁵ is H.

(32) In another embodiment of this invention W is —C(O)—, U is CR⁵, and R⁵ is alkyl (e.g. methyl).

(33) In another embodiment of this invention W is —C(O)—, U is CR⁵, and G is —C(R³)(R⁴)—.

(34) In another embodiment of this invention W is —C(O)—, U is CR⁵, G is —C(R³)(R⁴)—, R³ is H, and R⁴ is H.

(35) In another embodiment of this invention W is —C(O)—, U is CR⁵, G is —C(R³)(R⁴)—, R³ and R⁴ are each independently selected from the group consisting of: H and alkyl (e.g. methyl).

(36) In another embodiment of this invention W is —C(O)—, U is CR⁵, R⁵ is H, and G is —C(R³)(R⁴)—.

(37) In another embodiment of this invention W is —C(O)—, U is CR⁵, R⁵ is H, G is —C(R³)(R⁴)—, R³ is H, and R⁴ is H.

(38) In another embodiment of this invention W is —C(O)—, U is CR⁵, R⁵ is H, G is —C(R³)(R⁴)—, R³ and R⁴ are each independently selected from the group consisting of: H and alkyl (e.g. methyl).

(39) In another embodiment of this invention W is —C(O)—, U is CR⁵, R⁵ is alkyl (e.g. methyl), and G is —C(R³)(R⁴)—.

(40) In another embodiment of this invention W is —C(O)—, U is CR⁵, R⁵ is alkyl (e.g. methyl), G is —C(R³)(R⁴)—, R³ is H, and R⁴ is H.

(41) In another embodiment of this invention W is —C(O)—, U is CR⁵, R⁵ is alkyl (e.g. methyl), G is —C(R³)(R⁴)—, R³ and R⁴ are each independently selected from the group consisting of: H and alkyl (e.g. methyl).

(42) In another embodiment of this invention W is —C(O)—, U is N, G is —C(O)—.

(43) In another embodiment of this invention W is —C(O)—, U is CR⁵, and G is —C(O)—.

(44) In another embodiment of this invention W is —C(O)—, U is CR⁵, R⁵ is H, and G is —C(O)—.

(45) In another embodiment of this invention W is —C(O—), U is CR⁵, R⁵ is alkyl (e.g. methyl), and G is —C(O)—.

(46) In another embodiment of this invention W is —C(O)—, U is CR⁵, R⁵ is alkyl (e.g. methyl), and G is —C(O)—.

(47) In another embodiment of this invention W is —C(O)—, U is N, and G is —N(R¹³)—.

(48) In another embodiment of this invention W is —C(O)—, U is CR⁵, and G is —N(R¹³)—.

(49) In another embodiment of this invention W is —C(O)—, U is CR⁵, R⁵ is H, and G is —N(R¹³)—.

(50) In another embodiment of this invention W is —C(O)—, U is CR⁵, R⁵ is alkyl (e.g. methyl), and G is —N(R¹³)—.

(51) In another embodiment of this invention W is —C(O)—, U is N, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(52) In another embodiment of this invention W is —C(O)—, U is CR⁵, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(53) In another embodiment of this invention W is —C(O)—, U is CR⁵, R⁵ is H, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(54) In another embodiment of this invention W is —C(O)—, U is CR⁵, R⁵ is alkyl (e.g. methyl), G is —N(R¹³)—, and R¹³ is H.

(55) In another embodiment of this invention W is —C(O)—, U is N, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(56) In another embodiment of this invention W is —C(O)—, U is CR⁵, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(57) In another embodiment of this invention W is —C(O)—, U is CR⁵, R⁵ is H, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(58) In another embodiment of this invention W is —C(O)—, U is CR⁵, R⁵ is alkyl (e.g. methyl), G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(59) In one embodiment of this invention W is —S(O)—.

(60) In another embodiment of this invention W is —S(O)— and U is N.

(61) In another embodiment of this invention W is —S(O)—, U is N, and G is —C(R³)(R⁴)—.

(62) In another embodiment of this invention W is —S(O)—, U is N, G is —C(R³)(R⁴)—, R³ is H, and R⁴ is H.

(63) In another embodiment of this invention W is —S(O)—, U is N, G is —C(R³)(R⁴)—, and R³ and R⁴ are each independently selected from the group consisting of: H and alkyl (e.g. methyl).

(64) In another embodiment of this invention W is —S(O)— and U is CR⁵.

(65) In another embodiment of this invention W is —S(O)—, U is CR⁵, and R⁵ is H.

(66) In another embodiment of this invention W is —S(O)—, U is CR⁵, and R⁵ is alkyl (e.g. methyl).

(67) In another embodiment of this invention W is —S(O)—, U is CR⁵, and G is —C(R³)(R⁴)—.

(68) In another embodiment of this invention W is —S(O)—, U is CR⁵, G is —C(R³)(R⁴)—, R³ is H, and R⁴ is H.

(69) In another embodiment of this invention W is —S(O)—, U is CR⁵, G is —C(R³)(R⁴)—, R³ and R⁴ are each independently selected from the group consisting of: H and alkyl (e.g. methyl).

(70) In another embodiment of this invention W is —S(O)—, U is CR⁵, R⁵ is H, and G is —C(R³)(R⁴)—.

(71) In another embodiment of this invention W is —S(O)—, U is CR⁵, R⁵ is H, G is —C(R³)(R⁴)—, R³ is H, and R⁴ is H.

(72) In another embodiment of this invention W is —S(O)—, U is CR⁵, R⁵ is H, G is —C(R³)(R⁴)—, R³ and R⁴ are each independently selected from the group consisting of: H and alkyl (e.g. methyl).

(73) In another embodiment of this invention W is —S(O)—, U is CR⁵, R⁵ is alkyl (e.g. methyl), and G is —C(R³)(R⁴)—.

(74) In another embodiment of this invention W is —S(O)—, U is CR⁵, R⁵ is alkyl (e.g. methyl), G is —C(R³)(R⁴)—, R³ is H, and R⁴ is H.

(75) In another embodiment of this invention W is —S(O)—, U is CR⁵, R⁵ is alkyl (e.g. methyl), G is —C(R³)(R⁴)—, R³ and R⁴ are each independently selected from the group consisting of: H and alkyl (e.g. methyl).

(76) In another embodiment of this invention W is —S(O)—, U is N, and G is —N(R¹³)—.

(77) In another embodiment of this invention W is —S(O)—, U is CR⁵, and G is —N(R¹³)—.

(78) In another embodiment of this invention W is —S(O)—, U is CR⁵, R⁵ is H, and G is —N(R¹³)—.

(79) In another embodiment of this invention W is —S(O)—, U is CR⁵, R⁵ is alkyl (e.g. methyl), and G is —N(R¹³)—.

(80) In another embodiment of this invention W is —S(O)—, U is N, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(81) In another embodiment of this invention W is —S(O)—, U is CR⁵, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(82) In another embodiment of this invention W is —S(O)—, U is CR⁵, R⁵ is H, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(83) In another embodiment of this invention W is —S(O)—, U is CR⁵, R⁵ is alkyl (e.g. methyl), G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(84) In another embodiment of this invention W is —S(O)—, U is N, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(85) In another embodiment of this invention W is —S(O)—, U is CR⁵, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(86) In another embodiment of this invention W is —S(O)—, U is CR⁵, R⁵ is H, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(87) In another embodiment of this invention W is —S(O)—, U is CR⁵, R⁵ is alkyl (e.g. methyl), G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(88) In one embodiment of this invention W is —S(O₂)—.

(89) In another embodiment of this invention W is —S(O₂)— and U is N.

(90) In another embodiment of this invention W is —S(O₂)—, U is N, and G is —C(R³)(R⁴)—.

(91) In another embodiment of this invention W is —S(O₂)—, U is N, G is —C(R³)(R⁴)—, R³ is H, and R⁴ is H.

(92) In another embodiment of this invention W is —S(O₂)—, U is N, G is —C(R³)(R⁴)—, and R³ and R⁴ are independently selected from the group consisting of: H and alkyl (e.g. methyl).

(93) In another embodiment of this invention W is —S(O₂)— and U is CR⁵.

(94) In another embodiment of this invention W is —S(O₂)—, U is CR⁵, and R⁵ is H.

(95) In another embodiment of this invention W is —S(O₂)—, U is CR⁵, and R⁵ is alkyl (e.g. methyl).

(96) In another embodiment of this invention W is —S(O₂)—, U is CR⁵, and G is —C(R³)(R⁴)—.

(97) In another embodiment of this invention W is —S(O₂)—, U is CR⁵, G is —C(R³)(R⁴)—, R³ is H, and R⁴ is H.

(98) In another embodiment of this invention W is —S(O₂)—, U is CR⁵, G is —C(R³)(R⁴)—, R³ and R⁴ are each independently selected from the group consisting of: H and alkyl (e.g. methyl).

(99) In another embodiment of this invention W is —S(O₂)—, U is CR⁵, R⁵ is H, and G is —C(R³)(R⁴).

(100) In another embodiment of this invention W is —S(O₂)—, U is CR⁵, R⁵ is H, G is —C(R³)(R⁴)—, R³ is H, and R⁴ is H.

(101) In another embodiment of this invention W is —S(O₂)—, U is CR⁵, R⁵ is H, G is —C(R³)(R⁴)—, R³ and R⁴ are each independently selected from the group consisting of: H and alkyl (e.g. methyl).

(102) In another embodiment of this invention W is —S(O₂)—, U is CR⁵, R⁵ is alkyl (e.g. methyl), and G is —C(R³)(R⁴)—.

(103) In another embodiment of this invention W is —S(O₂)—, U is CR⁵, R⁵ is alkyl (e.g. methyl), G is —C(R³)(R⁴)—, R³ is H, and R⁴ is H.

(104) In another embodiment of this invention W is —S(O₂)—, U is CR⁵, R⁵ is alkyl (e.g. methyl), G is —C(R³)(R⁴)—, R³ and R⁴ are each independently selected from the group consisting of: H and alkyl (e.g. methyl).

(105) In another embodiment of this invention W is S(O₂), U is N, G is —N(R¹³)—.

(106) In another embodiment of this invention W is S(O₂), U is CR⁵, and G is —N(R¹³)—.

(107) In another embodiment of this invention W is S(O₂), U is CR⁵, R⁵ is H, and G is —N(R¹³)—.

(108) In another embodiment of this invention W is S(O₂), U is CR⁵, R⁵ is alkyl (e.g. methyl), and G is —N(R¹³)—.

(109) In another embodiment of this invention W is S(O₂), U is N, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(110) In another embodiment of this invention W is S(O₂), U is CR⁵, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(111) In another embodiment of this invention W is S(O₂), U is CR⁵, R⁵ is H, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(112) In another embodiment of this invention W is S(O₂), U is CR⁵, R⁵ is alkyl (e.g. methyl), G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(113) In another embodiment of this invention W is S(O₂), U is N, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(114) In another embodiment of this invention W is S(O₂), U is CR⁵, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(115) In another embodiment of this invention W is S(O₂), U is CR⁵, R⁵ is H, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(116) In another embodiment of this invention W is S(O₂), U is CR⁵, R⁵ is alkyl (e.g. methyl), G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(117) In one embodiment of this invention W is —C(R¹¹)(R¹²)—.

(118) In one embodiment of this invention W is —C(R¹¹)(R¹²)—, R¹¹ is H, and R¹² is H.

(119) In one embodiment of this invention W is —C(R¹¹)(R¹²)—, and R¹¹ and R¹² are each independently selected from the group consisting of: H and alkyl (e.g., methyl).

(120) In another embodiment of this invention W is —C(R¹¹)(R¹²)— and U is N.

(121) In another embodiment of this invention W is —C(R¹¹)(R¹²)—, U is N, R¹¹ is H, and R¹² is H.

(122) In another embodiment of this invention W is —C(R¹¹)(R¹²)—, U is N, and

R¹¹ and R¹² are each independently selected from the group consisting of: H and alkyl (e.g., methyl).

(123) In another embodiment of this invention W is —C(R¹¹)(R¹²)—, U is N, and G is —C(R³)(R⁴)—.

(124) In another embodiment of this invention W is —C(R¹¹)(R¹²)—, U is N, G is —C(R³)(R⁴)—, R¹¹ is H, and R¹² is H.

(125) In another embodiment of this invention W is —C(R¹¹)(R¹²)—, U is N, G is —C(R³)(R⁴)—, and R¹¹ and R¹² are each independently selected from the group consisting of: H and alkyl (e.g., methyl).

(126) In another embodiment of this invention W is —C(R¹¹)(R¹²)—, U is N, G is —C(R³)(R⁴)—, R³ is H, and R⁴ is H.

(127) In another embodiment of this invention W is —C(R¹¹)(R¹²)—, U is N, G is —C(R³)(R⁴)—, R³ is H, R⁴ is H, R¹¹ is H, and R¹² is H.

(128) In another embodiment of this invention W is —C(R¹¹)(R¹²)—, U is N, G is —C(R³)(R⁴)—, R³ is H, R⁴ is H, and R¹¹ and R¹² are each independently selected from the group consisting of: H and alkyl (e.g., methyl).

(129) In another embodiment of this invention W is —C(R¹¹)(R¹²)—, U is N, G is —C(R³)(R⁴)—, and R³ and R⁴ are independently selected from the group consisting of: H and alkyl (e.g. methyl).

(130) In another embodiment of this invention W is —C(R¹¹)(R¹²)—, U is N, G is —C(R³)(R⁴)—, R³ and R⁴ are independently selected from the group consisting of: H and alkyl (e.g. methyl), R¹¹ is H, and R¹² is H.

(131) In another embodiment of this invention W is —C(R¹¹)(R¹²)—, U is N, G is —C(R³)(R⁴)—, R³ and R⁴ are independently selected from the group consisting of: H and alkyl (e.g. methyl), and R¹¹ and R¹² are each independently selected from the group consisting of: H and alkyl (e.g., methyl).

(132) In another embodiment of this invention W is —C(R¹¹)(R¹²)— and U is CR⁵.

In another embodiment of this invention W is —C(R¹¹)(R¹²)—, U is CR⁵, R¹¹ is H, and R¹² is H.

(133) In another embodiment of this invention W is —C(R¹¹)(R¹²)—, U is CR⁵, and R¹¹ and R¹² are each independently selected from the group consisting of: H and alkyl (e.g., methyl).

(134) In another embodiment of this invention W is —C(R¹¹)(R¹²)—, U is CR⁵, and R⁵ is H.

(135) In another embodiment of this invention W is —C(R¹¹)(R¹²)—, U is CR⁵, R⁵ is H, R¹¹ is H, and R¹² is H.

(136) In another embodiment of this invention W is —C(R¹¹)(R¹²)—, U is CR⁵, R⁵ is H, and R¹¹ and R¹² are each independently selected from the group consisting of: H and alkyl (e.g., methyl).

(137) In another embodiment of this invention W is —C(R¹¹)(R¹²)—, U is CR⁵, and R⁵ is alkyl (e.g. methyl).

(138) In another embodiment of this invention W is —C(R¹¹)(R¹²)—, U is CR⁵, R⁵ is alkyl (e.g. methyl), R¹¹ is H, and R¹² is H.

(139) In another embodiment of this invention W is —C(R¹¹)(R¹²)—, U is CR⁵, R⁵ is alkyl (e.g. methyl), and R¹¹ and R¹² are each independently selected from the group consisting of: H and alkyl (e.g., methyl).

(140) In another embodiment of this invention W is —C(R¹¹)(R¹²)—, U is CR⁵, and G is —C(R³)(R⁴)—.

(141) In another embodiment of this invention W is —C(R¹¹)(R¹²)—, U is CR⁵, G is —C(R³)(R⁴)—, R¹¹ is H, and R¹² is H.

(142) In another embodiment of this invention W is —C(R¹¹)(R¹²)—, U is CR⁵, G is —C(R³)(R⁴)—, and R¹¹ and R¹² are each independently selected from the group consisting of: H and alkyl (e.g., methyl).

(143) In another embodiment of this invention W is —C(R¹¹)(R¹²)—, U is CR⁵, G is —C(R³)(R⁴), R³ is H, and R⁴ is H.

(144) In another embodiment of this invention W is —C(R¹¹)(R¹²)—, U is CR⁵, G is —C(R³)(R⁴), R³ is H, R⁴ is H, R¹¹ is H, and R¹² is H.

(145) In another embodiment of this invention W is —C(R¹¹)(R¹²)—, U is CR⁵, G is —C(R³)(R⁴), R³ is H, R⁴ is H, and R¹¹ and R¹² are each independently selected from the group consisting of: H and alkyl (e.g., methyl).

(146) In another embodiment of this invention W is —C(R¹¹)(R¹²)—, U is CR⁵, G is —C(R³)(R⁴)—, and R³ and R⁴ are each independently selected from the group consisting of: H and alkyl (e.g. methyl).

(147) In another embodiment of this invention W is —C(R¹¹)(R¹²)—, U is CR⁵, G is —C(R³)(R⁴)—, R³ and R⁴ are each independently selected from the group consisting of: H and alkyl (e.g. methyl), R¹¹ is H, and R¹² is H.

(148) In another embodiment of this invention W is —C(R¹¹)(R¹²)—, U is CR⁵, G is —C(R³)(R⁴)—, R³ and R⁴ are each independently selected from the group consisting of: H and alkyl (e.g. methyl), and R¹¹ and R¹² are each independently selected from the group consisting of: H and alkyl (e.g., methyl).

(149) In another embodiment of this invention W is —C(R¹¹)(R¹²)—, U is CR⁵, R⁵ is H, and G is —C(R³)(R⁴)—.

(150) In another embodiment of this invention W is —C(R¹¹)(R¹²)—, U is CR⁵, R⁵ is H, G is —C(R³)(R⁴)—, R¹¹ is H, and R¹² is H.

(151) In another embodiment of this invention W is —C(R¹¹)(R¹²)—, U is CR⁵, R⁵ is H, G is —C(R³)(R⁴)—, and R¹¹ and R¹² are each independently selected from the group consisting of: H and alkyl (e.g., methyl).

(152) In another embodiment of this invention W is —C(R¹¹)(R¹²)—, U is CR⁵, R⁵ is H, G is —C(R³)(R⁴)—, R³ is H, and R⁴ is H.

(153) In another embodiment of this invention W is —C(R¹¹)(R¹²)—, U is CR⁵, R⁵ is H, G is —C(R³)(R⁴)—, R³ is H, R⁴ is H, R¹¹ is H, and R¹² is H.

(154) In another embodiment of this invention W is —C(R¹¹)(R¹²)—, U is CR⁵, R⁵ is H, G is —C(R³)(R⁴)—, R³ is H, R⁴ is H, and R¹¹ and R¹² are each independently selected from the group consisting of: H and alkyl (e.g., methyl).

(155) In another embodiment of this invention W is —C(R¹²)—, U is CR⁵, R⁵ is H, G is —C(R³)(R⁴)—, and R³ and R⁴ are each independently selected from the group consisting of: H and alkyl (e.g. methyl).

(156) In another embodiment of this invention W is —C(R¹¹)(R¹²)—, U is CR⁵, R⁵ is H, G is —C(R³)(R⁴)—, R³ and R⁴ are each independently selected from the group consisting of: H and alkyl (e.g. methyl), R¹¹ is H, and R¹² is H.

(157) In another embodiment of this invention W is —C(R¹¹)(R¹²)—, U is CR⁵, R⁵ is H, G is —C(R³)(R⁴)—, R³ and R⁴ are each independently selected from the group consisting of: H and alkyl (e.g. methyl), and R¹¹ and R¹² are each independently selected from the group consisting of: H and alkyl (e.g., methyl).

(158) In another embodiment of this invention W is —C(R¹¹)(R¹²)—, U is CR⁵, R⁵ is alkyl (e.g. methyl), and G is —C(R³)(R⁴)—.

(159) In another embodiment of this invention W is —C(R¹¹)(R¹²)—, U is CR⁵, R⁵ is alkyl (e.g. methyl), G is —C(R³)(R⁴)—, R¹¹ is H, and R¹² is H.

(160) In another embodiment of this invention W is —C(R¹¹)(R¹²)—, U is CR⁵, R⁵ is alkyl (e.g. methyl), G is —C(R³)(R⁴)—, and R¹¹ and R¹² are each independently selected from the group consisting of: H and alkyl (e.g., methyl).

(161) In another embodiment of this invention W is —C(R¹¹)(R¹²)—, U is CR⁵, R⁵ is alkyl (e.g. methyl), G is —C(R³)(R⁴)—, R³ is H, and R⁴ is H.

(162) In another embodiment of this invention W is —C(R¹¹)(R¹²)—, U is CR⁵, R⁵ is alkyl (e.g. methyl), G is —C(R³)(R⁴)—, R³ is H, R⁴ is H, R¹¹ is H, and R¹² is H.

(163) In another embodiment of this invention W is —C(R¹¹)(R¹²)—, U is CR⁵, R⁵ is alkyl (e.g. methyl), G is —C(R³)(R⁴)—, R³ is H, R⁴ is H, and R¹¹ and R¹² are each independently selected from the group consisting of: H and alkyl (e.g., methyl).

(164) In another embodiment of this invention W is —C(R¹¹)(R¹²)—, U is CR⁵, R⁵ is alkyl (e.g. methyl), G is —C(R³)(R⁴)—, and R³ and R⁴ are each independently selected from the group consisting of: H and alkyl (e.g. methyl).

(165) In another embodiment of this invention W is —C(R¹¹)(R¹²)—, U is CR⁵, R⁵ is alkyl (e.g. methyl), G is —C(R³)(R⁴)—, R³ and R⁴ are each independently selected from the group consisting of: H and alkyl (e.g. methyl), R¹¹ is H, and R¹² is H.

(166) In another embodiment of this invention W is —C(R¹¹)(R¹²)—, U is CR⁵, R⁵ is alkyl (e.g. methyl), G is —C(R³)(R⁴)—, R³ and R⁴ are each independently selected from the group consisting of: H and alkyl (e.g. methyl), and R¹¹ and R¹² are each independently selected from the group consisting of: H and alkyl (e.g., methyl).

(167) In another embodiment of this invention W is —C(R¹¹)(R¹²)—, U is N, and G is —C(O)—.

(168) In another embodiment of this invention W is —C(R¹¹)(R¹²)—, U is N, G is —C(O)—, R¹¹ is H, and R¹² is H.

(169) In another embodiment of this invention W is —C(R¹¹)(R¹²)—, U is N, G is —C(O)—, and R¹¹ and R¹² are each independently selected from the group consisting of: H and alkyl (e.g., methyl).

(170) In another embodiment of this invention W is —C(R¹¹)(R¹²)—, U is CR⁵, and G is —C(O)—.

(171) In another embodiment of this invention W is —C(R¹¹)(R¹²)—, U is CR⁵, G is —C(O)—, R¹¹ is H, and R¹² is H.

(172) In another embodiment of this invention W is —C(R¹¹)(R¹²)—, U is CR⁵, G is —C(O)—, and R¹¹ and R¹² are each independently selected from the group consisting of: H and alkyl (e.g., methyl).

(173) In another embodiment of this invention W is —C(R¹¹)(R¹²)—, U is CR⁵, R⁵ is H, and G is —C(O)—.

(174) In another embodiment of this invention W is —C(R¹¹)(R¹²)—, U is CR⁵, R⁵ is H, G is —C(O)—, R¹¹ is H, and R¹² is H.

(175) In another embodiment of this invention W is —C(R¹¹)(R¹²)—, U is CR⁵, R⁵ is H, G is —C(O)—, and R¹¹ and R¹² are each independently selected from the group consisting of: H and alkyl (e.g., methyl).

(176) In another embodiment of this invention W is —C(R¹¹)(R¹²)—, U is CR⁵, R⁵ is alkyl (e.g. methyl), and G is —C(O)—.

(177) In another embodiment of this invention W is —C(R¹¹)(R¹²)—, U is CR⁵, R⁵ is alkyl (e.g. methyl), G is —C(O)—, R¹¹ is H, and R¹² is H.

(178) In another embodiment of this invention W is —C(R¹¹)(R¹²)—, U is CR⁵, R⁵ is alkyl (e.g. methyl), G is —C(O)—, and R¹¹ and R¹² are each independently selected from the group consisting of: H and alkyl (e.g., methyl).

(179) In another embodiment of this invention W is —C(R¹¹)(R¹²)—, U is N, and G is —N(R¹³)—.

(180) In another embodiment of this invention W is —C(R¹¹)(R¹²)—, U is N, G is —N(R¹³)—, R¹¹ is H, and R¹² is H.

(181) In another embodiment of this invention W is —C(R¹¹)(R¹²)—, U is N, G is —N(R¹³)—, and R¹¹ and R¹² are each independently selected from the group consisting of: H and alkyl (e.g., methyl).

(182) In another embodiment of this invention W is —C(R¹¹)(R¹²)—, U is CR⁵, and G is —N(R¹³)—.

(183) In another embodiment of this invention W is —C(R¹¹)(R¹²)—, U is CR⁵, G is —N(R¹³)—, R¹¹ is H, and R¹² is H.

(184) In another embodiment of this invention W is —C(R¹¹)(R¹²)—, U is CR⁵, G is —N(R¹³)—, and R¹¹ and R¹² are each independently selected from the group consisting of: H and alkyl (e.g., methyl).

(185) In another embodiment of this invention W is —C(R¹¹)(R¹²)—, U is CR⁵, R⁵ is H, and G is —N(R¹³)—.

(186) In another embodiment of this invention W is —C(R¹¹)(R¹²)—, U is CR⁵, R⁵ is H, G is —N(R¹³)—, R¹¹ is H, and R¹² is H.

(187) In another embodiment of this invention W is —C(R¹¹)(R¹²)—, U is CR⁵, R⁵ is H, G is —N(R¹³)—, and R¹¹ and R¹² are each independently selected from the group consisting of: H and alkyl (e.g., methyl).

(188) In another embodiment of this invention W is —C(R¹¹)(R¹²)—, U is CR⁵, R⁵ is alkyl (e.g. methyl), and G is —N(R¹³)—.

(189) In another embodiment of this invention W is —C(R¹¹)(R¹²)—, U is CR⁵, R⁵ is alkyl (e.g. methyl), G is —N(R¹³)—, R¹¹ is H, and R¹² is H.

(190) In another embodiment of this invention W is —C(R¹¹)(R¹²)—, U is CR⁵, R⁵ is alkyl (e.g. methyl), G is —N(R¹³)—, and R¹¹ and R¹² are each independently selected from the group consisting of: H and alkyl (e.g., methyl).

(191) In another embodiment of this invention W is —C(R¹¹)(R¹²)—, U is N, G is —N(R¹³)—, and R¹³ is H.

(192) In another embodiment of this invention W is —C(R¹¹)(R¹²)—, U is N, G is —N(R¹³)—, R¹³ is H, R¹¹ is H, and R¹² is H.

(193) In another embodiment of this invention W is —C(R¹¹)(R¹²)—, U is N, G is —N(R¹³)—, R¹³ is H, and R¹¹ and R¹² are each independently selected from the group consisting of: H and alkyl (e.g., methyl).

(194) In another embodiment of this invention W is —C(R¹¹)(R¹²)—, U is CR⁵, G is —N(R¹³)—, and R¹³ is H.

(195) In another embodiment of this invention W is —C(R¹¹)(R¹²)—, U is CR⁵, G is —N(R¹³)—, R¹³ is H, R¹¹ is H, and R¹² is H.

(196) In another embodiment of this invention W is —C(R¹¹)(R¹²)—, U is CR⁵, G is —N(R¹³)—, R¹³ is H, and R¹¹ and R¹² are each independently selected from the group consisting of: H and alkyl (e.g., methyl).

(197) In another embodiment of this invention W is —C(R¹¹)(R¹²)—, U is CR⁵, R⁵ is H, G is —N(R¹³)—, and R¹³ is H.

(198) In another embodiment of this invention W is —C(R¹¹)(R¹²)—, U is CR⁵, R⁵ is H, G is —N(R¹³)—, R¹³ is H, R¹¹ is H, and R¹² is H.

(199) In another embodiment of this invention W is —C(R¹¹)(R¹²)—, U is CR⁵, R⁵ is H, G is —N(R¹³)—, R¹³ is H, and R¹¹ and R¹² are each independently selected from the group consisting of: H and alkyl (e.g., methyl).

(200) In another embodiment of this invention W is —C(U is CR⁵, R⁵ is alkyl (e.g. methyl), G is —N(R¹³)—, and R¹³ is H.

(201) In another embodiment of this invention W is —C(R¹¹)(R¹²)—, U is CR⁵, R⁵ is alkyl (e.g. methyl), G is —N(R¹³)—, R¹³ is H, R¹¹ is H, and R¹² is H.

(202) In another embodiment of this invention W is —C(R¹¹)(R¹²)—, U is CR⁵, R⁵ is alkyl (e.g. methyl), G is —N(R¹³)—, R¹³ is H, and R¹¹ and R¹² are each independently selected from the group consisting of: H and alkyl (e.g., methyl).

(203) In another embodiment of this invention W is —C(R¹¹)(R¹²)—, U is N, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(204) In another embodiment of this invention W is —C(R¹¹)(R¹²)—, U is N, G is —N(R¹³)—, R¹¹ is H, and R¹² is H, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(205) In another embodiment of this invention W is —C(R¹¹)(R¹²)—, U is N, G is —N(R¹³)—, R¹¹ and R¹² are each independently selected from the group consisting of: H and alkyl (e.g., methyl), and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(206) In another embodiment of this invention W is —C(R¹¹)(R¹²)—, U is CR⁵, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(207) In another embodiment of this invention W is —C(R¹¹)(R¹²)—, U is CR⁵, G is —N(R¹³)—, R¹¹ is H, and R¹² is H, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(208) In another embodiment of this invention W is —C(R¹¹)(R¹²)—, U is CR⁵, G is —N(R¹³)—, R¹¹ and R¹² are each independently selected from the group consisting of: H and alkyl (e.g., methyl), and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(209) In another embodiment of this invention W is —C(R¹¹)(R¹²)—, U is CR⁵, R⁵ is H, G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(210) In another embodiment of this invention W is —C(R¹¹)(R¹²)—, U is CR⁵, R⁵ is H, G is —N(R¹³)—, R¹¹ is H, and R¹² is H, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(211) In another embodiment of this invention W is —C(R¹¹)(R¹²)—, U is CR⁵, R⁵ is H, G is —N(R¹³)—, R¹¹ and R¹² are each independently selected from the group consisting of: H and alkyl (e.g., methyl), and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(212) In another embodiment of this invention W is —C(R¹¹)(R¹²)—, U is CR⁵, R⁵ is alkyl (e.g. methyl), G is —N(R¹³)—, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(213) In another embodiment of this invention W is —C(R¹¹)(R¹²)—, U is CR⁵, R⁵ is alkyl (e.g. methyl), G is —N(R¹³)—, R¹¹ is H, and R¹² is H, and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(214) In another embodiment of this invention W is —C(R¹¹)(R¹²)—, U is CR⁵, R⁵ the group consisting of: H and alkyl (e.g., methyl), and R¹³ is selected from the group consisting of: H, alkyl, cycloalkyl, phenyl, and phenyl substituted with 1 to 5 (e.g., 1 to 3) independently selected R²¹ substituents. In one example, R¹³ is H.

(215) Other embodiments are directed to any one of the embodiments described in any one of the paragraphs numbered (1) to (214) above wherein B is a five membered ring.

(216) Other embodiments are directed to any one of the embodiments described in any one of the paragraphs numbered (1) to (214) above wherein B is a five membered ring, and the optional bond is present.

(217) Other embodiments are directed to any one of the embodiments described in any one of the paragraphs numbered (1) to (214) above wherein B is a five membered ring, and the optional bond is absent.

(218) Other embodiments are directed to any one of the embodiments described in any one of the paragraphs numbered (1) to (214) above wherein B is a six membered ring.

(219) Other embodiments are directed to any one of the embodiments described in any one of the paragraphs numbered (1) to (214) above wherein B is a six membered ring, and the optional bond is present.

(220) Other embodiments are directed to any one of the embodiments described in any one of the paragraphs numbered (1) to (214) above wherein B is a six membered ring, and the optional bond is absent.

(221) Other embodiments are directed to any one of the embodiments described in any one of the paragraphs numbered (1) to (214) above wherein B is a seven membered ring.

(222) Other embodiments are directed to any one of the embodiments described in any one of the paragraphs numbered (1) to (214) above wherein B is a seven membered ring, and the optional bond is present.

(223) Other embodiments are directed to any one of the embodiments described in any one of the paragraphs numbered (1) to (214) above wherein B is a seven membered ring, and the optional bond is absent.

(224) Other embodiments are directed to any one of the embodiments described in any one of the paragraphs numbered (1) to (214) above wherein B is an eight membered ring.

(225) Other embodiments are directed to any one of the embodiments described in any one of the paragraphs numbered (1) to (214) above wherein B is an eight membered ring, and the optional bond is present.

(226) Other embodiments are directed to any one of the embodiments described in any one of the paragraphs numbered (1) to (214) above wherein B is an eight membered ring, and the optional bond is absent.

(227) Other embodiments are directed to any one of the embodiments described in any one of the paragraphs numbered (1) to (214) above wherein B is a five membered ring, and there are 1 or 2 heteroatoms present in ring B (not including U when U is N, that is the 1 or 2 heteroatoms in ring B are in addition to U when U is N).

(228) Other embodiments are directed to any one of the embodiments described in any one of the paragraphs numbered (1) to (214) above wherein B is a five membered ring, and there are 1 or 2 heteroatoms present in ring B (not including U when U is N, that is the 1 or 2 heteroatoms in ring B are in addition to U when U is N), and the optional bond is present.

(229) Other embodiments are directed to any one of the embodiments described in any one of the paragraphs numbered (1) to (214) above wherein B is a five membered ring, and there are 1 or 2 heteroatoms present in ring B (not including U when U is N, that is the 1 or 2 heteroatoms in ring B are in addition to U when U is N), and the optional bond is absent.

(230) Other embodiments are directed to any one of the embodiments described in any one of the paragraphs numbered (1) to (214) above wherein B is a six membered ring, and there are 1 or 2 heteroatoms present in ring B (not including U when U is N, that is the 1 or 2 heteroatoms in ring B are in addition to U when U is N).

(231) Other embodiments are directed to any one of the embodiments described in any one of the paragraphs numbered (1) to (214) above wherein B is a six membered ring, and there are 1 or 2 heteroatoms present in ring B (not including U when U is N, that is the 1 or 2 heteroatoms in ring B are in addition to U when U is N), and the optional bond is present.

(232) Other embodiments are directed to any one of the embodiments described in any one of the paragraphs numbered (1) to (214) above wherein B is a six membered ring, and there are 1 or 2 heteroatoms present in ring B (not including U when U is N, that is the 1 or 2 heteroatoms in ring B are in addition to U when U is N), and the optional bond is absent.

(233) Other embodiments are directed to any one of the embodiments described in any one of the paragraphs numbered (1) to (214) above wherein B is a seven membered ring, and there are 1 or 2 heteroatoms present in ring B (not including U when U is N, that is the 1 or 2 heteroatoms in ring B are in addition to U when U is N).

(234) Other embodiments are directed to any one of the embodiments described in any one of the paragraphs numbered (1) to (214) above wherein B is a seven membered ring, and there are 1 or 2 heteroatoms present in ring B (not including U when U is N, that is the 1 or 2 heteroatoms in ring B are in addition to U when U is N), and the optional bond is present.

(235) Other embodiments are directed to any one of the embodiments described in any one of the paragraphs numbered (1) to (214) above wherein B is a seven membered ring, and there are 1 or 2 heteroatoms present in ring B (not including U when U is N, that is the 1 or 2 heteroatoms in ring B are in addition to U when U is N), and the optional bond is absent.

(236) Other embodiments are directed to any one of the embodiments described in any one of the paragraphs numbered (1) to (214) above wherein B is an eight membered ring, and there are 1 or 2 heteroatoms present in ring B (not including U when U is N, that is the 1 or 2 heteroatoms in ring B are in addition to U when U is N).

(237) Other embodiments are directed to any one of the embodiments described in any one of the paragraphs numbered (1) to (214) above wherein B is an eight membered ring, and there are 1 or 2 heteroatoms present in ring B (not including U when U is N, that is the 1 or 2 heteroatoms in ring B are in addition to U when U is N), and the optional bond is present.

(238) Other embodiments are directed to any one of the embodiments described in any one of the paragraphs numbered (1) to (214) above wherein B is an eight membered ring, and there are 1 or 2 heteroatoms present in ring B (not including U when U is N, that is the 1 or 2 heteroatoms in ring B are in addition to U when U is N), and the optional bond is absent.

(239) Other embodiments are directed to any one of the embodiments described in any one of the paragraphs numbered (1) to (214) above wherein B is a five membered ring, and there is 1 heteroatom present in ring B (not including U when U is N, that is the 1 heteroatom in ring B is in addition to U when U is N).

(240) Other embodiments are directed to any one of the embodiments described in any one of the paragraphs numbered (1) to (214) above wherein there is 1 heteroatom present in ring B (not including U when U is N, that is the 1 heteroatom in ring B is in addition to U when U is N), and the optional bond is present.

(241) Other embodiments are directed to any one of the embodiments described in any one of the paragraphs numbered (1) to (214) above wherein B is a five membered ring, and there is 1 heteroatom present in ring B (not including U when U is N, that is the 1 heteroatom in ring B is in addition to U when U is N), and the optional bond is absent.

(242) Other embodiments are directed to any one of the embodiments described in any one of the paragraphs numbered (1) to (214) above wherein B is a six membered ring, and there is 1 heteroatom present in ring B (not including U when U is N, that is the 1 heteroatom in ring B is in addition to U when U is N).

(243) Other embodiments are directed to any one of the embodiments described in any one of the paragraphs numbered (1) to (214) above wherein B is a six membered ring, and there is 1 heteroatom present in ring B (not including U when U is N, that is the 1 heteroatom in ring B is in addition to U when U is N), and the optional bond is present.

(244) Other embodiments are directed to any one of the embodiments described in any one of the paragraphs numbered (1) to (214) above wherein B is a six membered ring, and there is 1 heteroatom present in ring B (not including U when U is N, that is the 1 heteroatom in ring B is in addition to U when U is N), and the optional bond is absent.

(245) Other embodiments are directed to any one of the embodiments described in any one of the paragraphs numbered (1) to (214) above wherein B is a seven membered ring, and there is 1 heteroatom present in ring B (not including U when U is N, that is the 1 heteroatom in ring B is in addition to U when U is N).

(246) Other embodiments are directed to any one of the embodiments described in any one of the paragraphs numbered (1) to (214) above wherein B is a seven membered ring, and there is 1 heteroatom present in ring B (not including U when U is N, that is the 1 heteroatom in ring B is in addition to U when U is N), and the optional bond is present.

(247) Other embodiments are directed to any one of the embodiments described in any one of the paragraphs numbered (1) to (214) above wherein B is a seven membered ring, and there is 1 heteroatom present in ring B (not including U when U is N, that is the 1 heteroatom in ring B is in addition to U when U is N), and the optional bond is absent.

(248) Other embodiments are directed to any one of the embodiments described in any one of the paragraphs numbered (1) to (214) above wherein B is an eight membered ring, and there is 1 heteroatom present in ring B (not including U when U is N, that is the 1 heteroatom in ring B is in addition to U when U is N).

(249) Other embodiments are directed to any one of the embodiments described in any one of the paragraphs numbered (1) to (214) above wherein B is an eight membered ring, and there is 1 heteroatom present in ring B (not including U when U is N, that is the 1 heteroatom in ring B is in addition to U when U is N), and the optional bond is present.

(250) Other embodiments are directed to any one of the embodiments described in any one of the paragraphs numbered (1) to (214) above wherein B is an eight membered ring, and there is 1 heteroatom present in ring B (not including U when U is N, that is the 1 heteroatom in ring B is in addition to U when U is N), and the optional bond is absent.

In one embodiment R² (of the NR² moiety) is H.

In another embodiment R² (of the NR² moiety) is alkyl, such as, for example, methyl, ethyl or isopropyl.

In another embodiment R² (of the NR² moiety) is aryl, such as, for example, phenyl.

In another embodiment R² (of the NR² moiety) is substituted aryl, such as, for example, substituted phenyl.

In another embodiment R² (of the NR² moiety) is —C(O)R^{4A} wherein R^{4A} is alkyl (such as, for example, methyl, ethyl or isopropyl).

In another embodiment R² (of the NR² moiety) is —C(O)R^{4A} wherein R^{4A} is aryl, such as, for example, phenyl. moiety is selected from the group consisting of: H, alkyl (such as, for example, methyl, ethyl or isopropyl), (aryl, such as, for example, phenyl), —C(O)R^{4A} wherein R^{4A} is alkyl (such as, for example, methyl, ethyl or isopropyl), and —C(O)R^{4A} wherein R^{4A} is substituted aryl, such as, for example, substituted phenyl)

In another embodiment of this invention R⁵ is H.

In another embodiment of this invention ring (B) is not substituted with any R²¹ groups. Thus, other embodiments of this invention are directed to any one of the embodiments described in paragraphs (1) to (250) above wherein ring (B) is not substituted with any R²¹ groups.

In another embodiment of this invention ring (B) is substituted with 1 to 5 independently selected R²¹ groups. Thus, other embodiments of this invention are directed to any one of the embodiments described in paragraphs (1) to (250) above wherein ring (B) is substituted with 1 to 5 independently selected R²¹ groups.

In another embodiment of this invention, there are 1 to 5 R²¹ groups present in formula (I), and at least one (e.g., 1 to 2) R²¹ is selected from the group consisting of: —SF₅, —OSF₅ and —Si(R^{15A})₃, wherein each R¹⁵ is independently selected.

In another embodiment of this invention, there are 1 to 5 R²¹ groups present in formula (I), and at least one R²¹ is selected from the group consisting of: —SF₅ and —Si(R^{15A})₃, and each R¹⁵ is the same or different alkyl group.

In another embodiment of this invention, there are 1 to 5 R²¹ groups present in formula (I), and at least one R²¹ is selected from the group consisting of: —SF₅, —OSF₅ and —Si(CH₃)₃.

In another embodiment of this invention, there are 1 to 5 R²¹ groups present in formula (I), and one of the R²¹ groups is selected from the group consisting of: —SF₅, OSF₅ and —Si(R^{15A})₃.

In another embodiment of this invention, there are 1 to 5 R²¹ groups present in formula (I), and one of the R²¹ groups is selected from the group consisting of: —SF₅, OSF₅ and —Si(R^{15A})₃, and each R^{15A} is the same or different alkyl group.

In another embodiment of this invention, there are 1 to 5 R²¹ groups present in formula (I), and one of the R²¹ groups is selected from the group consisting of: —SF₅, —OSF₅ and —Si(CH₃)₃.

In another embodiment of this invention, there are 2 to 5 R²¹ groups present in formula (I), and two of the R²¹ groups are selected from the group consisting of: —SF₅, OSF₅ and —Si(R^{15A})₃, wherein each R^{15A} is independently selected.

In another embodiment of this invention, there are 2 to 5 R²¹ groups present in formula (I), and two of the R²¹ groups are selected from the group consisting of: —SF₅, OSF₅ and —Si(R^{15A})₃, and each R^{15A} is the same or different alkyl group.

In another embodiment of this invention, there are 2 to 5 R²¹ groups present in formula (I), and two of the R²¹ groups are selected from the group consisting of: —SF₅, —OSF₅ and —Si(CH₃)₃.

In another embodiment of this invention, there are 1 to 5 R²¹ groups present in formula (I), and at least one (e.g., 1 to 2) R²¹ is selected from the group consisting of: —SF₅ and —Si(R^{15A})₃, wherein each R^{15A} is independently selected.

In another embodiment of this invention, there are 1 to 5 R²¹ groups present in formula (I), and at least one R²¹ is selected from the group consisting of: —SF₅ and —Si(R^{15A})₃, and each R^{15A} is the same or different alkyl group.

In another embodiment of this invention, there are 1 to 5 R²¹ groups present in formula (I), and at least one R²¹ is selected from the group consisting of: —SF₅ and —Si(CH₃)₃.

In another embodiment of this invention, there are 1 to 5 R²¹ groups present in formula (I), and one of the R²¹ groups is selected from the group consisting of: —SF₅ and —Si(R^{15A})₃.

In another embodiment of this invention, there are 1 to 5 R²¹ groups present in formula (I), and one of the R²¹ groups is selected from the group consisting of: —SF₅ and —Si(R^{15A})₃, and each R¹⁵ is the same or different alkyl group.

In another embodiment of this invention, there are 1 to 5 R²¹ groups present in formula (I), and one of the R²¹ groups is selected from the group consisting of: —SF₅ and —Si(CH₃)₃.

In another embodiment of this invention, there are 2 to 5 R²¹ groups present in formula (I), and two of the R²¹ groups are selected from the group consisting of: —SF₅ and —Si(R^{15A})₃, wherein each R^{15A} is independently selected.

In another embodiment of this invention, there are 2 to 5 R²¹ groups present in formula (I), and two of the R²¹ groups are selected from the group consisting of: —SF₅ and —Si(R^{15A})₃, and each R¹⁵ is the same or different alkyl group.

In another embodiment of this invention, there are 2 to 5 R²¹ groups present in formula (I), and two of the R²¹ groups are selected from the group consisting of: —SF₅ and —Si(CH₃)₃.

In another embodiment of this invention, there are 1 to 5 R²¹ groups present in formula (I), and one of the R²¹ groups is —SF₅.

In another embodiment of this invention, there are 2 to 5 R²¹ groups present in formula (I), and two of the R²¹ groups are —SF₅.

In another embodiment of this invention, there are 1 to 5 R²¹ groups present in formula (I), and one of the R²¹ groups is —OSF₅.

In another embodiment of this invention, there are 2 to 5 R²¹ groups present in formula (I), and two of the R²¹ groups are —OSF₅.

In another embodiment of this invention, there are 1 to 5 R²¹ groups present in formula (I), and one of the R²¹ groups is —Si(R^{15A})₃.

In another embodiment of this invention, there are 1 to 5 R²¹ groups present in formula (I), and one of the R²¹ groups is —Si(R^{15A})₃ and each R^{15A} is the same or different alkyl group.

In another embodiment of this invention, there are 1 to 5 R²¹ groups present in formula (I), and one of the R²¹ groups is —Si(CH₃)₃.

In another embodiment of this invention, there are 2 to 5 R²¹ groups present in formula (I), and two of the R²¹ groups are the same or different —Si(R^{15A})₃, wherein each R^{15A} is independently selected.

In another embodiment of this invention, there are 2 to 5 R²¹ groups present in formula (I), and two of the R²¹ groups are the same or different —Si(R^{15A})₃ and each R^{15A} is the same or different alkyl group.

In another embodiment of this invention, there are 2 to 5 R²¹ groups present in formula (I), and two of the R²¹ groups are —Si(CH₃)₃.

In another embodiment of this invention R⁷ is substituted with R²¹ groups, and at least one (e.g. 1 to 2) of the R²¹ groups is selected from the group consisting of: —SF₅, —OSF₅ and —Si(R^{15A})₃, wherein each R^{15A} is independently selected.

In another embodiment of this invention R⁷ is substituted with R²¹ groups, and at least one (e.g. 1 to 2) of the R²¹ groups is selected from the group consisting of: —SF₅, —OSF₅ and —Si(R^{15A})₃, and each R^{15A} is the same or different alkyl group.

In another embodiment of this invention R⁷ is substituted with R²¹ groups, and at least one (e.g. 1 to 2) of the R²¹ groups is selected from the group consisting of: —SF₅, —OSF₅ and —Si(CH₃)₃.

In another embodiment of this invention R⁷ is substituted with R²¹ groups, and one R²¹ group is selected from the group consisting of: —SF₅, —OSF₅ and —Si(R^{15A})₃, wherein each R^{15A} is independently selected.

In another embodiment of this invention R⁷ is substituted with R²¹ groups, and one R²¹ group is selected from the group consisting of: —SF₅, —OSF₅ and —Si(R^{15A})₃, and each R^{15A} is the same or different alkyl group.

In another embodiment of this invention R⁷ is substituted with R²¹ groups, and one R²¹ group is selected from the group consisting of: —SF₅, —OSF₅ and —Si(CH₃)₃.

In another embodiment of this invention R⁷ is substituted with R²¹ groups, and two R²¹ groups are selected from the group consisting of: —SF₅, —OSF₅ and —Si(R^{15A})₃, wherein each R^{15A} is independently selected.

In another embodiment of this invention R⁷ is substituted with R²¹ groups, and two R²¹ groups are selected from the group consisting of: —SF₅, —OSF₅ and —Si(R^{15A})₃, and each R^{15A} is the same or different alkyl group.

In another embodiment of this invention R⁷ is substituted with R²¹ groups, and two R²¹ groups are selected from the group consisting of: —SF₅, —OSF₅ and —Si(CH₃)₃.

In another embodiment of this invention R⁷ is substituted with R²¹ groups, and one R²¹ group is —SF₅.

In another embodiment of this invention R⁷ is substituted with R²¹ groups, and two R²¹ groups are —SF₅.

In another embodiment of this invention R⁷ is substituted with R²¹ groups, and one R²¹ group is —OSF₅.

In another embodiment of this invention R⁷ is substituted with R²¹ groups, and two R²¹ groups are —OSF₅.

In another embodiment of this invention R⁷ is substituted with R²¹ groups, and one R²¹ group is —Si(R^{15A})₃, wherein each R^{15A} is independently selected.

In another embodiment of this invention R⁷ is substituted with R²¹ groups, and one R²¹ group is —Si(R^{15A})₃ and each R^{15A} is the same or different alkyl group.

In another embodiment of this invention R⁷ is substituted with R²¹ groups, and one R²¹ group is —Si(CH₃)₃.

In another embodiment of this invention R⁷ is substituted with R²¹ groups, and two of the R²¹ groups are the same or different —Si(R^{15A})₃, wherein each R^{15A} is independently selected.

In another embodiment of this invention R⁷ is substituted with R²¹ groups, and two of the R²¹ groups are the same or different —Si(R^{15A})₃ group, and each R^{15A} is the same or different alkyl group.

In another embodiment of this invention R⁷ is substituted with R²¹ groups, and two of the R²¹ group are —Si(CH₃)₃.

In another embodiment of this invention R⁷ is an aryl group substituted with R²¹ groups, and at least one (e.g., 1 to 2) R²¹ group is selected from the group consisting of: —SF₅, —OSF₅ and —Si(R^{15A})₃, wherein each R^{15A} is independently selected.

In another embodiment of this invention R⁷ is an aryl group group substituted with R²¹ groups, and at least one (e.g., 1 to 2) R²¹ group is selected from the group consisting of: —SF₅, —OSF₅ and —Si(R^{15A})₃, and each R^{15A} is the same or different alkyl group.

In another embodiment of this invention R⁷ is an aryl group substituted with R²¹ groups, and at least one (e.g., 1 to 2) R²¹ group is selected from the group consisting of: —SF₅, —OSF₅ and —Si(CH₃)₃.

In another embodiment of this invention R⁷ is an aryl group substituted with R²¹ groups, and said aryl moiety is phenyl, and at least one (e.g., 1 to 2) R²¹ group is selected from the group consisting of: —SF₅, —OSF₅ and —Si(R^{15A})₃, wherein each R^{15A} is independently selected.

In another embodiment of this invention R⁷ is an aryl group substituted with R²¹ groups, and said aryl moiety is phenyl, and at least one (e.g., 1 to 2) R²¹ group is selected from the group consisting of: —SF₅, —OSF₅ and —Si(R^{15A})₃, and each R^{15A} is the same or different alkyl group.

In another embodiment of this invention R⁷ is an aryl group substituted with R²¹ groups, and said aryl moiety is phenyl, and at least one (e.g., 1 to 2) R²¹ group is selected from the group consisting of: —SF₅, —OSF₅ and —Si(CH₃)₃.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R^{15A})₃, wherein each R^{15A} is independently selected.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R^{15A})₃, and each R^{15A} is the same or different alkyl group.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(CH₃)₃.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R^{15A})₃, wherein each R^{15A} is independently selected.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R^{15A})₃, and each R^{15A} is the same or different alkyl group.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(CH₃)₃.

In another embodiment of this invention R⁷ is an aryl group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R^{15A})₃, wherein each R^{15A} is independently selected.

In another embodiment of this invention R⁷ is an aryl group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R^{15A})₃, and each R^{15A} is the same or different alkyl group.

In another embodiment of this invention R⁷ is an aryl group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(CH₃)₃.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —SF₅.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —OSF₅.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —Si(R^{15A})₃, wherein each R^{15A} is independently selected.

In another embodiment of this invention R⁷ is an aryl group group substituted with R²¹ groups, and said aryl moiety is phenyl, and said phenyl is substituted with at least one (e.g., 1 to 3, or 1 to 2) R²¹ group, and one R²¹ group on said phenyl is —Si(R^{15A})₃, and each R^{15A} is the same or different alkyl group.

In another embodiment of this invention R⁷ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —Si(CH₃)₃.

In another embodiment of this invention R⁷ is an aryl group substituted with R²¹ groups, and said aryl moiety is phenyl, and said phenyl is substituted with at least two (e.g., 2 to 3) R²¹ groups, and two of the R²¹ groups on said phenyl are —SF₅.

In another embodiment of this invention R⁷ is an aryl group substituted with R²¹ groups, and said aryl moiety is phenyl, and said phenyl is substituted with at least two (e.g., 2 to 3) R²¹ groups, and two of the R²¹ groups on said phenyl are —OSF₅.

In another embodiment of this invention R⁷ is an aryl group substituted with R²¹ groups, and said aryl moiety is phenyl, and said phenyl is substituted with at least two (e.g., 2 to 3) R²¹ groups, and two of the R²¹ groups on said phenyl are —Si(R^{15A})₃, wherein each R^{15A} is independently selected.

In another embodiment of this invention R⁷ is an aryl group substituted with R²¹ groups, and said aryl moiety is phenyl, and said phenyl is substituted with at least two (e.g., 2 to 3) R²¹ groups, and two of the R²¹ groups on said phenyl are —Si(R^{15A})₃, and each R^{15A} is the same or different alkyl group.

In another embodiment of this invention R⁷ is an aryl group substituted with R²¹ groups, and said aryl moiety is phenyl, and said phenyl is substituted with at least two (e.g., 2 to 3) R²¹ groups, and two of the R²¹ groups on said phenyl are —Si(CH₃)₃.

In another embodiment of this invention R⁶ is alkyl.

In another embodiment of this invention R⁶ is a C₁ to C₃ alkyl group.

In another embodiment of this invention R⁶ is methyl.

In another embodiment of this invention R⁶ is ethyl.

In another embodiment of this invention R⁶ is a C₃ alkyl group.

In another embodiment of this invention R⁶ is isopropyl.

In another embodiment R⁶ is —C(O)OR¹⁵.

In another embodiment R⁶ is —C(O)OR¹⁵ wherein R¹⁵ is alkyl.

In another embodiment R⁶ is —C(O)OR¹⁵ wherein R¹⁵ is methyl.

In another embodiment R⁶ is alkyl substituted with 1-5 R²¹ groups.

In another embodiment R⁶ is alkyl substituted with one R²¹ group.

In another embodiment R⁶ is alkyl substituted with one R²¹ group, and said R²¹ group is —OR¹⁵.

In another embodiment R⁶ is alkyl substituted with one R²¹ group, and said R²¹ group is —OR¹⁵, and said R¹⁵ is alkyl.

In another embodiment R⁶ is alkyl substituted with one R²¹ group, and said R²¹ group is —OR¹⁵, and said R¹⁵ is methyl.

In another embodiment R⁶ is —CH₂R²¹ (i.e. alkyl substituted with one R²¹ group, wherein said alkyl is —CH₂—).

In another embodiment R⁶ is —CH₂OR¹⁵ (i.e. alkyl substituted with one R²¹ group, wherein said alkyl is —CH₂—, and said R²¹ group is —OR¹⁵).

In another embodiment R⁶ is —CH₂OR¹⁵ (i.e. alkyl substituted with one R²¹ group, wherein said alkyl is —CH₂—, and said R²¹ group is —OR¹⁵), wherein said R¹⁵ group is alkyl.

In another embodiment R⁶ is —CH₂OR¹⁵ (i.e. alkyl substituted with one R²¹ group, wherein said alkyl is —CH₂—, and said R²¹ group is —OR¹⁵), wherein said R¹⁵ group is methyl.

In another embodiment R⁶ is —C(O)NR¹⁵R¹⁶.

In another embodiment R⁶ is —C(O)NR¹⁵R¹⁶ wherein R¹⁵ and R¹⁶ are each independently selected from the group consisting of: H and alkyl.

In another embodiment R⁶ is —C(O)NR¹⁵R¹⁶ wherein R¹⁵ and R¹⁶ are the same or different alkyl.

In another embodiment R⁶ is —C(O)NR¹⁵R¹⁶ wherein R¹⁵ and R¹⁶ are each independently selected from the group consisting of: H and methyl.

In another embodiment R⁶ is —C(O)NR¹⁵R¹⁶ wherein R¹⁵ and R¹⁶ are each methyl.

Other embodiments of this invention are directed to any one of the embodiments described in paragraphs (1) to (250) above wherein R⁶ is alkyl. In one such embodiment R⁶ is a C₁ to C₃ alkyl group. In another embodiment R⁶ is methyl.

In another embodiment R⁶ is ethyl. In another embodiment R⁶ is a C₃ alkyl group. In another embodiment R⁶ is isopropyl.

In another embodiment of this invention R⁷ is an unsubstituted aryl group (e.g., an unsubstituted phenyl group). Thus, in another embodiment R⁷ is phenyl.

In another embodiment of this invention R⁷ is a substituted aryl group (e.g., a substituted phenyl group). Thus, in another embodiment R⁷ is a substituted phenyl group.

In another embodiment of this invention R⁷ is an aryl group substituted with 1 to 3 independently selected R²¹ groups.

In another embodiment of this invention R⁷ is an aryl group substituted with one to 3 R²¹ groups, and each R²¹ group is the same or different halo.

In another embodiment of this invention R⁷ is an aryl group substituted with one to 3 R²¹ groups, and each R²¹ group is F.

In another embodiment of this invention R⁷ is phenyl, and said phenyl is substituted with one or more independently selected R²¹ groups.

In another embodiment of this invention R⁷ is phenyl, and said phenyl is substituted with 1 to 3 independently selected R²¹ groups.

In another embodiment of this invention R⁷ is phenyl, and said phenyl is substituted with 1 to 3 R²¹ groups, and each R²¹ group is the same or different halo.

In another embodiment of this invention R⁷ is phenyl, and said phenyl is substituted with three R²¹ halo groups, and each R²¹ group is the same or different halo.

In another embodiment of this invention R⁷ is phenyl, and said phenyl is substituted with two R²¹ halo groups, and each R²¹ group is the same or different halo.

In another embodiment of this invention R⁷ is phenyl, and said phenyl is substituted with one R²¹ halo group.

In another embodiment of this invention R⁷ is phenyl, and said phenyl is substituted with 1 to 3 F (i.e., said phenyl is substituted with 1 to 3 R²¹ groups, and said R²¹ groups are halo, and said halo is F).

In another embodiment of this invention R⁷ is phenyl, and said phenyl is substituted with one F (i.e., said phenyl is substituted with one R²¹ group, and said R²¹ group is halo, and said halo is F).

In another embodiment of this invention R⁷ is phenyl, and said phenyl is substituted with two F atoms (i.e., said phenyl is substituted with two R²¹ groups, and said R²¹ groups are halo, and said halo is F).

In another embodiment of this invention R⁷ is phenyl, and said phenyl is substituted with three F atoms (i.e., said phenyl is substituted with three R²¹ groups, and said R²¹ groups are halo, and said halo is F).

In another embodiment of this invention R⁷ is phenyl, and said phenyl is substituted with one —CN group.

In another embodiment of this invention R⁷ is phenyl, and said phenyl is substituted with one or two R²¹ alkyl groups (e.g. methyl groups), wherein each R²¹ group is substituted with 1 to 3 R²² halo groups (e.g. F groups).

In another embodiment of this invention R⁷ is phenyl, and said phenyl is substituted with one or two —CF₃ groups (i.e. there are one or two R²¹ alkyl groups (i.e. methyl groups) each substituted with 3 R²² halo (i.e. F) groups).

In another embodiment of this invention R⁷ is selected from the group consisting of:

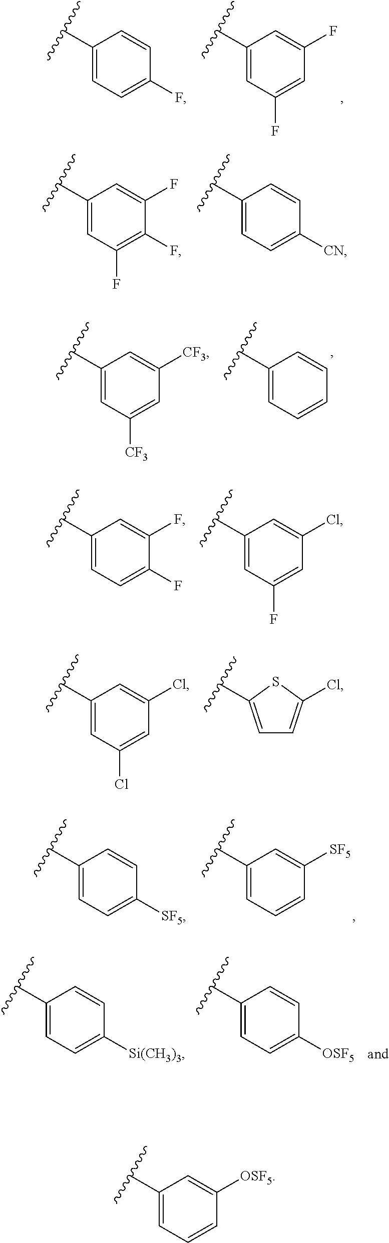

In another embodiment of this invention R⁷ is:

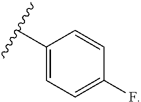

In another embodiment of this invention R⁷ is:

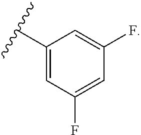

In another embodiment of this invention R⁷ is:

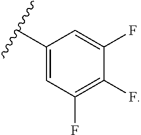

In another embodiment of this invention R⁷ is:

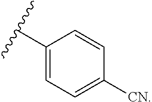

In another embodiment of this invention R⁷ is:

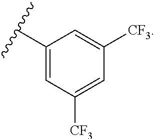

In another embodiment of this invention R⁷ is:

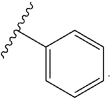

In another embodiment of this invention R⁷ is:

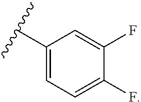

In another embodiment of this invention R⁷ is:

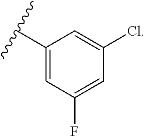

In another embodiment of this invention R⁷ is:

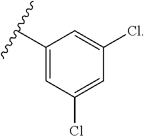

In another embodiment of this invention R⁷ is:

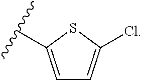

In another embodiment of this invention R⁷ is:

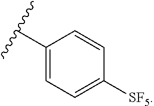

In another embodiment of this invention R⁷ is:

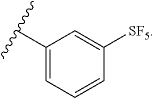

In another embodiment of this invention R⁷ is:

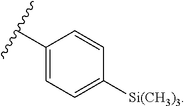

In another embodiment of this invention R⁷ is:

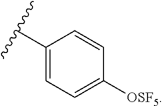

In another embodiment of this invention R⁷ is:

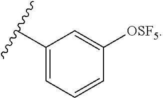

Other embodiments of this invention are directed to any one of the embodiments above directed to Ring (B) wherein:
(a) R⁶ is:
   (1) alkyl, or
   (2) C₁ to C₃ alkyl, or
   (3) methyl, or
   (4) ethyl, or
   (5) a C₃ alkyl group, or
   (6) isopropyl, or
   (7) —C(O)OR¹⁶, or
   (8) —C(O)OR¹⁶ wherein R¹⁵ is alkyl, or
   (9) —C(O)OR¹⁶ wherein R¹⁵ is methyl, or
   (10) alkyl substituted with 1-5 R²¹ groups, or
   (11) alkyl substituted with one R²¹ group, or
   (12) alkyl substituted with one R²¹ group, and said R²¹ group is —OR¹⁶, or
   (13) alkyl substituted with one R²¹ group, and said R²¹ group is —OR¹⁵, and said R¹⁵ is alkyl, or
   (14) alkyl substituted with one R²¹ group, and said R²¹ group is —OR¹⁵, and said R¹⁵ is methyl, or
   (15) —CH₂R²¹ (i.e. alkyl substituted with one R²¹ group, wherein said alkyl is —CH₂—), or
   (16) —CH₂OR¹⁵ (i.e. alkyl substituted with one R²¹ group, wherein said alkyl is —CH₂—, and said R²¹ group is —OR¹⁵), or
   (17) —CH₂OR¹⁵ (i.e. alkyl substituted with one R²¹ group, wherein said alkyl is —CH₂—, and said R²¹ group is —OR¹⁵), wherein said R¹⁵ group is alkyl, or
   (18) —CH₂OR¹⁵ (i.e. alkyl substituted with one R²¹ group, wherein said alkyl is —CH₂—, and said R²¹ group is —OR¹⁵), wherein said R¹⁵ group is methyl, or
   (19) —C(O)NR¹⁵R¹⁶, or
   (20) —C(O)NR¹⁵R¹⁶ wherein R¹⁵ and R¹⁶ are each independently selected from the group consisting of: H and alkyl, or
   (21) —C(O)NR¹⁵R¹⁶ wherein R¹⁵ and R¹⁶ are the same or different alkyl, or
   (22) —C(O)NR¹⁵R¹⁶ wherein R¹⁵ and R¹⁶ are each independently selected from the group consisting of: H and methyl, or
   (23) —C(O)NR¹⁵R¹⁶ wherein R¹⁵ and R¹⁶ are each methyl; and
(b) R⁷ is as defined in any one of the embodiments above that are directed to R⁷.

Other embodiments of this invention are directed to any one of the embodiments above directed to Ring (B) wherein:
(a) R⁶ is:
   (1) alkyl, or
   (2) C₁ to C₃ alkyl, or
   (3) methyl, or
   (4) ethyl, or
   (5) a C₃ alkyl group, or
   (6) isopropyl, or
   (7) —C(O)OR¹⁵, or
   (8) —C(O)OR¹⁵ wherein R¹⁵ is alkyl, or
   (9) —C(O)OR¹⁵ wherein R¹⁵ is methyl, or
   (10) alkyl substituted with 1-5 R²¹ groups, or
   (11) alkyl substituted with one R²¹ group, or
   (12) alkyl substituted with one R²¹ group, and said R²¹ group is —OR¹⁵, or
   (13) alkyl substituted with one R²¹ group, and said R²¹ group is —OR¹⁵, and said R¹⁵ is alkyl, or
   (14) alkyl substituted with one R²¹ group, and said R²¹ group is —OR¹⁵, and said R¹⁵ is methyl, or
   (15) —CH₂R²¹ (i.e. alkyl substituted with one R²¹ group, wherein said alkyl is —CH₂—), or
   (16) —CH₂OR¹⁵ (i.e. alkyl substituted with one R²¹ group, wherein said alkyl is —CH₂—, and said R²¹ group is —OR¹⁵), or
   (17) —CH₂OR¹⁵ (i.e. alkyl substituted with one R²¹ group, wherein said alkyl is —CH₂—, and said R²¹ group is —OR¹⁵), wherein said R¹⁵ group is alkyl, or
   (18) —CH₂OR¹⁵ (i.e. alkyl substituted with one R²¹ group, wherein said alkyl is —CH₂—, and said R²¹ group is —OR¹⁵), wherein said R¹⁵ group is methyl, or
   (19) —C(O)NR¹⁵R¹⁶, or
   (20) —C(O)NR¹⁵R¹⁶ wherein R¹⁵ and R¹⁶ are each independently selected from the group consisting of: H and alkyl, or
   (21) —C(O)NR¹⁵R¹⁶ wherein R¹⁵ and R¹⁶ are the same or different alkyl, or
   (22) —C(O)NR¹⁵R¹⁶ wherein R¹⁵ and R¹⁶ are each independently selected from the group consisting of: H and methyl, or
   (23) —C(O)NR¹⁵R¹⁶ wherein R¹⁵ and R¹⁶ are each methyl; and
(b) R⁷ is selected from the group consisting of:

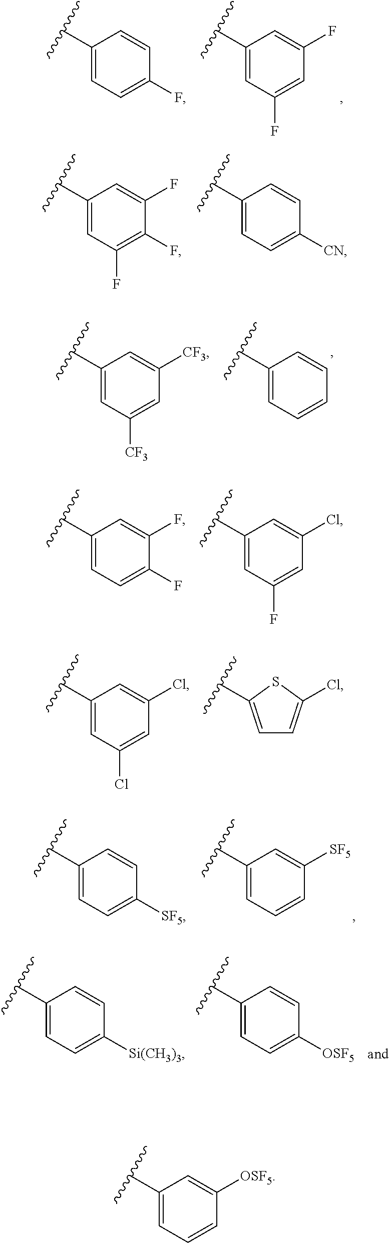

Other embodiments of this invention are directed to any one of the embodiments above directed to Ring (B) wherein:
(a) R⁶ is:
   (1) alkyl, or
   (2) C₁ to C₃ alkyl, or
   (3) methyl, or
   (4) ethyl, or
   (5) a C₃ alkyl group, or
   (6) isopropyl, or
   (7) —C(O)OR¹⁵, or
   (8) —C(O)OR¹⁵ wherein R¹⁵ is alkyl, or
   (9) —C(O)OR¹⁵ wherein R¹⁵ is methyl, or
   (10) alkyl substituted with 1-5 R²¹ groups, or
   (11) alkyl substituted with one R²¹ group, or
   (12) alkyl substituted with one R²¹ group, and said R²¹ group is —OR¹⁵, or
   (13) alkyl substituted with one R²¹ group, and said R²¹ group is —OR¹⁶, and said R¹⁵ is alkyl, or
   (14) alkyl substituted with one R²¹ group, and said R²¹ group is —OR¹⁵, and said R¹⁵ is methyl, or
   (15) —CH₂R²¹ (i.e. alkyl substituted with one R²¹ group, wherein said alkyl is —CH₂—), or
   (16) —CH₂OR¹⁵ (i.e. alkyl substituted with one R²¹ group, wherein said alkyl is —CH₂—, and said R²¹ group is —OR¹⁵), or
   (17) —CH₂OR¹⁵ (i.e. alkyl substituted with one R²¹ group, wherein said alkyl is —CH₂—, and said R²¹ group is —OR¹⁵), wherein said R¹⁵ group is alkyl, or
   (18) —CH₂OR¹⁵ (i.e. alkyl substituted with one R²¹ group, wherein said alkyl is —CH₂—, and said R²¹ group is —OR¹⁵), wherein said R¹⁵ group is methyl, or
   (19) —C(O)NR¹⁵R¹⁶, or
   (20) —C(O)NR¹⁵R¹⁶ wherein R¹⁵ and R¹⁶ are each independently selected from the group consisting of: H and alkyl, or
   (21) —C(O)NR¹⁵R¹⁶ wherein R¹⁵ and R¹⁶ are the same or different alkyl, or
   (22) —C(O)NR¹⁵R¹⁶ wherein R¹⁵ and R¹⁶ are each independently selected from the group consisting of: H and methyl, or
   (23) —C(O)NR¹⁵R¹⁶ wherein R¹⁵ and R¹⁶ are each methyl; and
(b) R⁷ is selected from the group consisting of:

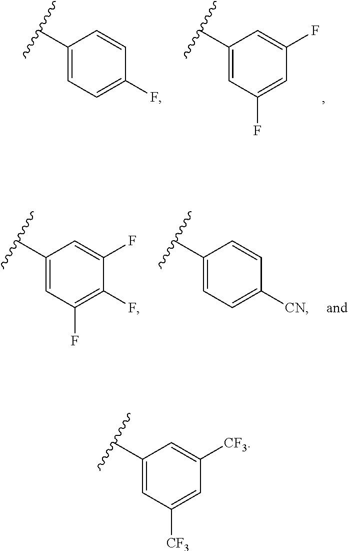

Other embodiments of this invention are directed to any one of the embodiments described in paragraphs (1) to (250) above wherein R⁷ is as described in any one of the above embodiments directed to R⁷.

Other embodiments of this invention are directed to any one of the embodiments described in paragraphs (1) to (250) above wherein R⁶ is alkyl and R⁷ is as defined in any one of the above embodiments directed to R⁷. In one such embodiment R⁶ is a C₁ to C₃ alkyl group. In another embodiment R⁶ is methyl. In another embodiment R⁶ is ethyl. In another embodiment R⁶ is a C₃ alkyl group. In another embodiment R⁶ is isopropyl.

In another embodiment of this invention R¹⁰ is selected from the group consisting of aryl and aryl substituted with one or more R²¹ groups.

Other embodiments of this invention are directed to any one of the embodiments described in paragraphs (1) to (250) above wherein R¹⁰ is selected from the group consisting of aryl and aryl substituted with one or more R²¹ groups.

In another embodiment of this invention R⁹ is selected from the group consisting of heteroaryl and heteroaryl substituted with one or more R²¹ groups, and wherein each R²¹ is independently selected.

Other embodiments of this invention are directed to any one of the embodiments described in paragraphs (1) to (250) above wherein R⁹ is selected from the group consisting of heteroaryl and heteroaryl substituted with one or more R²¹ groups, and wherein each R²¹ is independently selected.

In another embodiment of this invention R¹⁰ is selected from the group consisting of aryl and aryl substituted with one or more R²¹ groups, and R⁹ is selected from the group consisting of heteroaryl and heteroaryl substituted with one or more R²¹ groups, and wherein each R²¹ is independently selected.

Other embodiments of this invention are directed to any one of the embodiments described in paragraphs (1) to (250) above wherein R¹⁰ is selected from the group consisting of aryl and aryl substituted with one or more R²¹ groups, and R⁹ is selected from the group consisting of heteroaryl and heteroaryl substituted with one or more R²¹ groups, and wherein each R²¹ is independently selected.

Other embodiments of this invention are directed to any one of the embodiments described in paragraphs (1) to (250) above wherein R⁶ is alkyl, R⁷ is as defined in any one of the above embodiments directed to R⁷, R¹⁰ is selected from the group consisting of aryl and aryl substituted with one or more independently selected R²¹ groups, and R⁹ is selected from the group consisting of heteroaryl and heteroaryl substituted with one or more independently selected R²¹ groups. In one such embodiment R⁶ is a C₁ to C₃ alkyl group. In another embodiment R⁶ is methyl. In another embodiment R⁶ is ethyl. In another embodiment R⁶ is a C₃ alkyl group. In another embodiment R⁶ is isopropyl.

In another embodiment of this invention R¹⁰ is heteroaryl (e.g. pyridyl).

Other embodiments of this invention are directed to any one of the embodiments described in paragraphs (1) to (250) above wherein R¹⁰ is heteroaryl (e.g. pyridyl).

In another embodiment of this invention R¹⁰ is heteroaryl substituted with one or more R²¹ groups (e.g. pyridyl substituted with one or more R²¹ groups).

Other embodiments of this invention are directed to any one of the embodiments described in paragraphs (1) to (250) above wherein R¹⁰ is heteroaryl substituted with one or more R²¹ groups (e.g. pyridyl substituted with one or more R²¹ groups).

In another embodiment of this invention R¹⁰ is selected from the group consisting of heteroaryl and heteroaryl substituted with one or more R²¹ groups, and

R⁹ group is selected from the group consisting of heteroaryl and heteroaryl substituted with one or more R²¹ groups, and wherein each R²¹ is independently selected.

Other embodiments of this invention are directed to any one of the embodiments described in paragraphs (1) to (250) above wherein R¹⁰ is selected from the group consisting of heteroaryl and heteroaryl substituted with one or more R²¹ groups, and R⁹ group is selected from the group consisting of heteroaryl and heteroaryl substituted with one or more R²¹ groups, and wherein each R²¹ is independently selected.

Other embodiments of this invention are directed to the compounds of formula (I) wherein R¹⁰ is heteroaryl or heteroaryl substituted with one or more R²¹ groups, and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Other embodiments of this invention are directed to any one of the embodiments described in paragraphs (1) to (250) above wherein R¹⁶ is heteroaryl or heteroaryl substituted with one or more R²¹ groups, and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Other embodiments of this invention are directed to any one of the embodiments described in paragraphs (1) to (250) above wherein R⁶ is alkyl, R⁷ is as defined in any one of the above embodiments directed to R⁷, R¹⁰ is selected from the group consisting of heteroaryl and heteroaryl substituted with one or more independently selected R²¹ groups, and R⁹ is selected from the group consisting of heteroaryl and heteroaryl substituted with one or more independently selected R²¹ groups. In one such embodiment R⁶ is a C₁ to C₃ alkyl group. In another embodiment R⁶ is methyl. In another embodiment R⁶ is ethyl. In another embodiment R⁶ is a C₃ alkyl group. In another embodiment R⁶ is isopropyl.

In another embodiment of this invention R¹⁰ is aryl.

In another embodiment of this invention R¹⁰ aryl is aryl and said aryl is phenyl.

In another embodiment of this invention R¹⁰ is aryl substituted with one or more R²¹ groups.

In another embodiment of this invention R¹⁰ is aryl substituted with one or more R²¹ groups, and said aryl is phenyl, i.e., said R¹⁰ group is phenyl substituted with one or more R²¹ groups.

In another embodiment of this invention R¹⁰ is phenyl substituted with one or more R²¹ groups, and each R²¹ group is the same or different —OR¹⁵ group.

In another embodiment of this invention R¹⁰ is phenyl substituted with one or more R²¹ groups, and each R²¹ group is the same or different —OR¹⁵ group, and said R¹⁵ is alkyl, and each alkyl is independently selected.

In another embodiment of this invention R¹⁰ is phenyl substituted with one R²¹ group, and said R²¹ group is —OR¹⁵, and said R¹⁵ is alkyl.

In another embodiment of this invention R¹⁰ is phenyl substituted with one R²¹ group, and said R²¹ group is —OR¹⁵, and said R¹⁵ is alkyl, and said alkyl is methyl.

In another embodiment of this invention R¹⁰ is phenyl substituted with one or more (e.g., one or two, or one) independently selected R²¹ halo groups.

In another embodiment of this invention R¹⁰ is phenyl substituted with one R²¹ group, and said R²¹ group is halo.

In another embodiment of this invention R¹⁰ is phenyl substituted with one R²¹ group, and said R²¹ group is F.

In another embodiment of this invention R¹⁰ is phenyl substituted with one R²¹ group and said R²¹ is an —OR¹⁵ group, and R¹⁵ is an (R¹⁸)ₙalkyl group, and R¹⁸ is halo, and n is 1 to 3, and each halo is independently selected.

In another embodiment of this invention R¹⁰ is phenyl substituted with one R²¹ group and said R²¹ is an —OR¹⁵ group, and R¹⁵ is an (R¹⁸)ₙalkyl group, and R¹⁸ is F, and n is 3.

In another embodiment of this invention R¹⁰ is phenyl substituted with one R²¹ group and said R²¹ is an —OR¹⁵ group, and R¹⁵ is an (R¹⁸)ₙalkyl group, and R¹⁸ is F, and n is 3, and the alkyl is methyl (i.e., the R²¹ substituent is —OCF₃).

In another embodiment of this invention R⁹ is heteroaryl.

In another embodiment of this invention R⁹ is heteroaryl substituted with one or more R²¹ groups.

In another embodiment of this invention R⁹ is heteroaryl substituted with one or more R²¹ groups, and said R²¹ groups are the same or different alkyl.

In another embodiment of this invention R⁹ is heteroaryl substituted with one R²¹ group, and said R²¹ is alkyl.

In another embodiment of this invention R⁹ is heteroaryl substituted with one R²¹ group, and said R²¹ is alkyl, and said alkyl is methyl.

In another embodiment of this invention R⁹ is and said heteroaryl is imidazoyl.

In another embodiment of this invention R⁹ is imidazolyl substituted with one or more R²¹ groups.

In another embodiment of this invention R⁹ is imidazolyl substituted with one or more R²¹ groups, and said R²¹ groups are the same or different alkyl.

In another embodiment of this invention R⁹ is imidazolyl substituted with one R²¹ group, and said R²¹ is alkyl.

In another embodiment of this invention R⁹ is imidazolyl substituted with one R²¹ group, and said R²¹ is alkyl, and said alkyl is methyl.

In another embodiment of this invention R¹⁰ is selected from the group consisting of aryl and aryl substituted with one or more R²¹ groups, and said R⁹ group is selected from the group consisting of heteroaryl and heteroaryl substituted with one or more R²¹ groups, wherein each R²¹ is independently selected.

In another embodiment of this invention R¹⁰ is phenyl substituted with one or more R²¹ groups, and said R⁹ is imidazolyl substituted with one or more R²¹ groups, wherein each R²¹ is independently selected.

In another embodiment of this invention R¹⁰ is phenyl substituted with one R²¹ group, and said R⁹ is imidazolyl substituted with one R²¹ group, wherein each R²¹ is independently selected.

In another embodiment of this invention R¹⁰ is phenyl substituted with one or more independently selected —OR¹⁵ groups, and said R⁹ is imidazolyl substituted with one or more independently selected alkyl groups.

In another embodiment of this invention R¹⁰ is phenyl substituted with one or more independently selected —OR¹⁵ groups, and said R⁹ is imidazolyl substituted with one or more independently selected alkyl groups, and each R¹⁵ is the same or different alkyl group.

In another embodiment of this invention R¹⁰ is phenyl substituted with one —OR¹⁵ group, and said R⁹ is imidazolyl substituted with one alkyl group.

In another embodiment of this invention R¹⁰ is phenyl substituted with one —OR¹⁵ group, and said R⁹ is imidazolyl substituted with one alkyl group, and R¹⁵ is alkyl, and wherein the R¹⁵ alkyl group, and the alkyl group on said imidazolyl are independently selected.

In another embodiment of this invention R¹⁰ is phenyl substituted with one —OR¹⁵ group, and said R⁹ is imidazolyl substituted with one methyl group, and R¹⁵ is methyl, and wherein the R¹⁵ alkyl group, and the alkyl group on said imidazolyl are independently selected.

Other embodiments of the compounds of formula (I) are directed to any one of the above embodiments wherein R⁹ is:

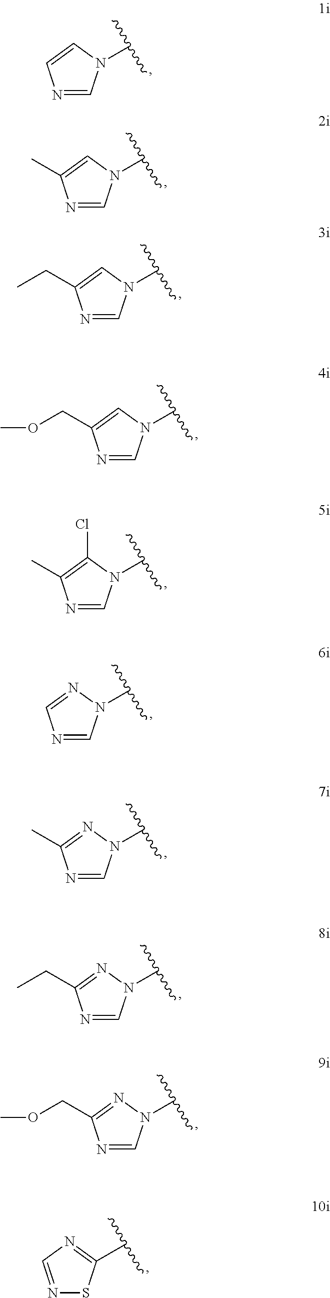

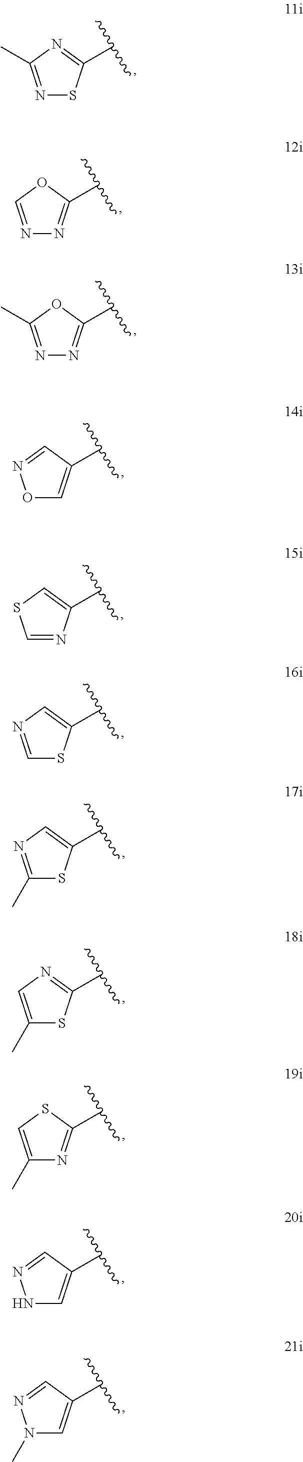

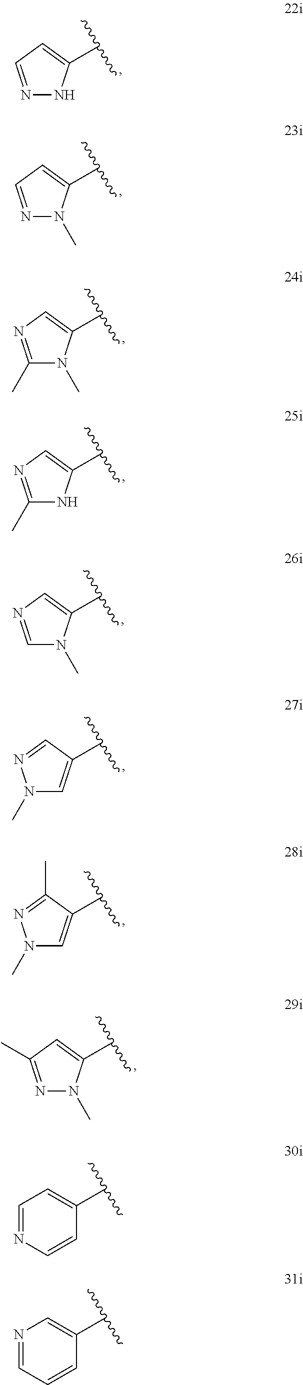

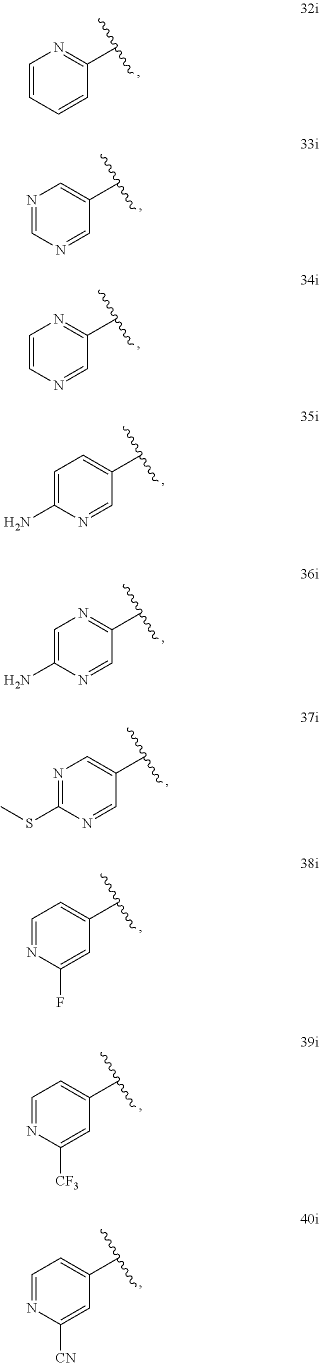

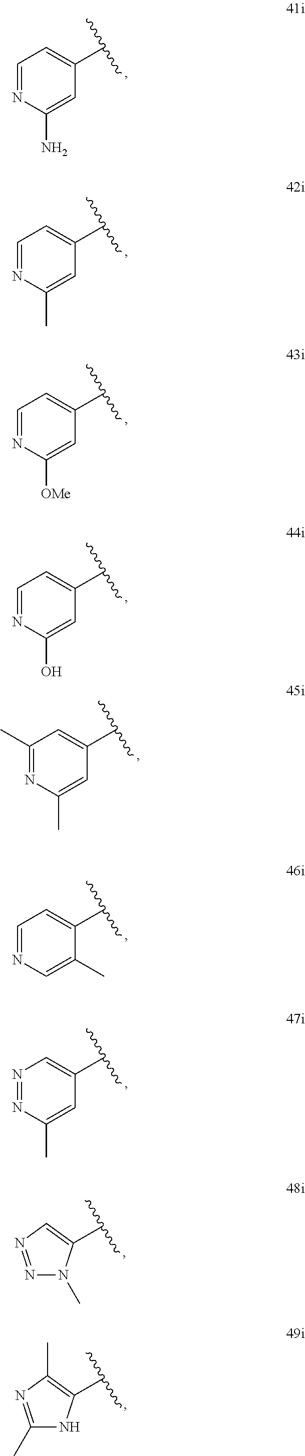

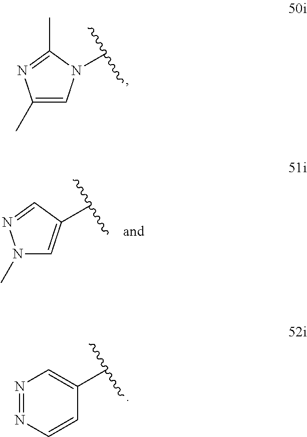

Other embodiments of the compounds of formula (I) are directed to any one of the above embodiments wherein R⁹ is:

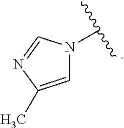

Other embodiments of the compounds of formula (I) are directed to any one of the above embodiments wherein R¹⁰ is:

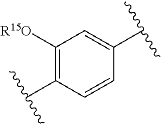

(wherein the —OR¹⁵ is ortho to the carbon to which R⁹ is bound to, i.e., the R⁹—R¹⁰-moiety is:

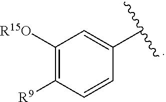

Other embodiments for the compounds of formula (I) are directed to any one of the above embodiments wherein R¹⁰ is:

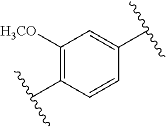

(wherein the —OCH₃ is ortho to the carbon to which R⁹ is bound to, i.e., the R⁹—R¹⁰-moiety is:

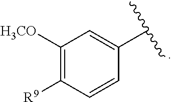

In another embodiment of this invention the R⁹—R¹⁰— moiety is:

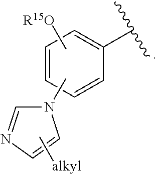

In another embodiment of this invention the R⁹—R¹⁰— moiety is:

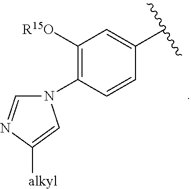

In another embodiment of this invention the R⁹—R¹⁰— moiety is:

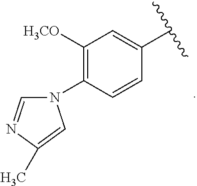

In another embodiment of this invention the R⁹—R¹⁰— moiety is:

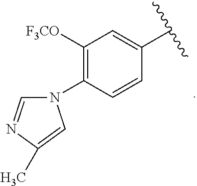

In another embodiment of this invention the R⁹—R¹⁰— moiety is:

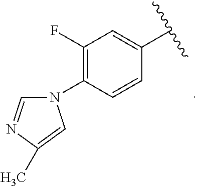

In another embodiment of this invention the R⁹—R¹⁰— moiety is:

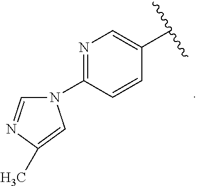

In another embodiment of this invention R⁷ is selected from the group consisting of:

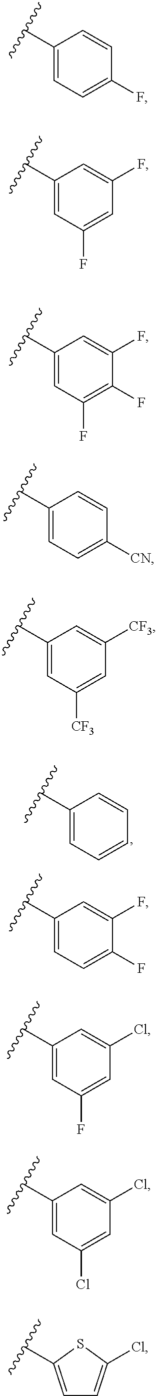

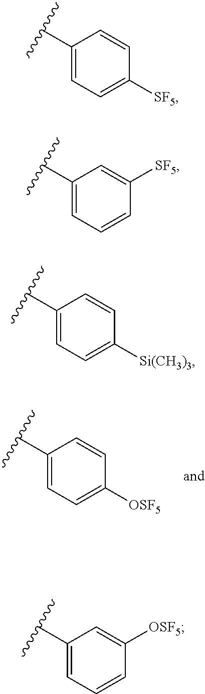

and
the R⁹—R¹⁰— moiety is selected from the group consisting of:

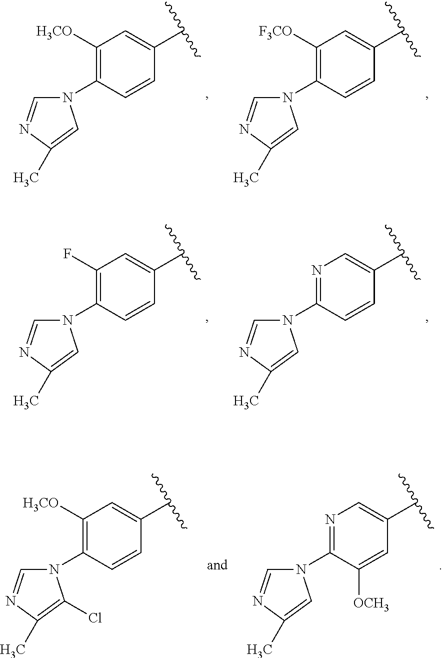

In another embodiment of this invention R⁷ is selected from the group consisting of:

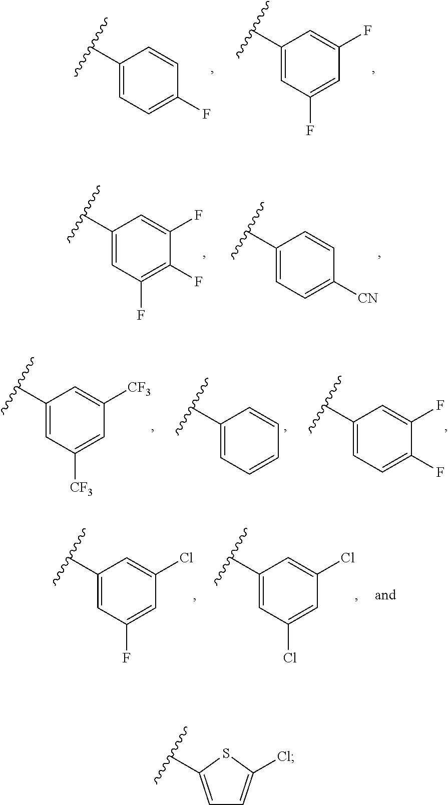

and
the R⁹—R¹⁰ moiety is selected from the group consisting of:

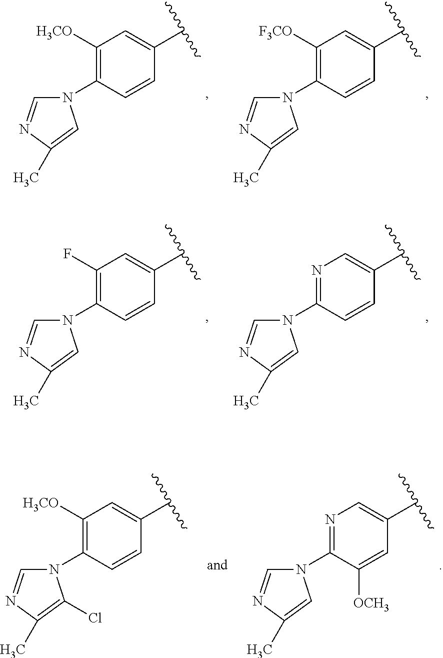

In another embodiment of this invention R⁷ is selected from the group consisting of:

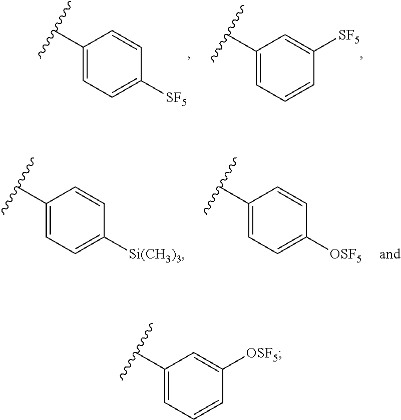

and
the R⁹—R¹⁰— moiety is selected from the group consisting of:

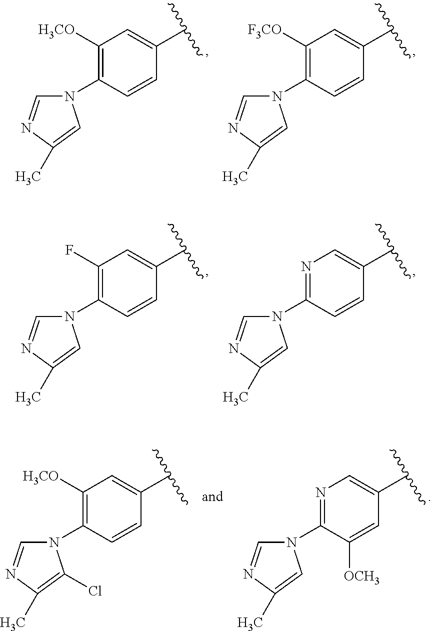

In another embodiment of this invention R⁷ is selected from the group consisting of:

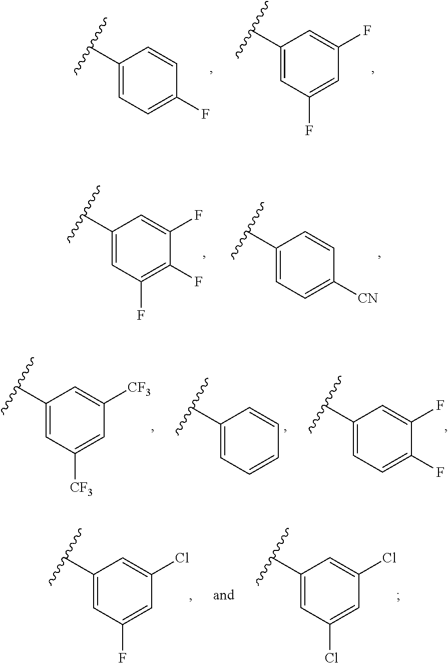

and
the R⁹—R¹⁰— moiety is selected from the group consisting of:

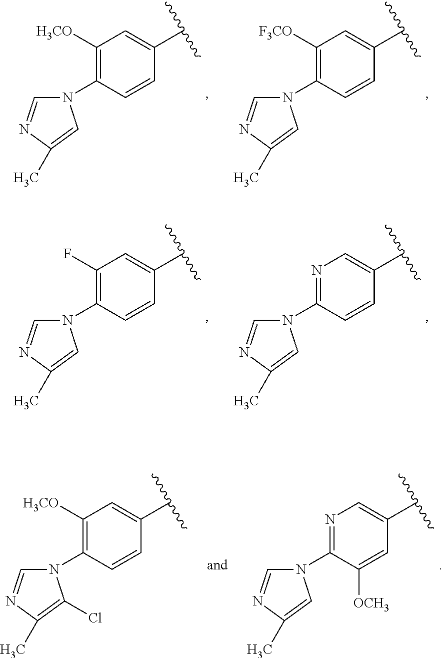

In another embodiment of this invention R⁷ is selected from the group consisting of:

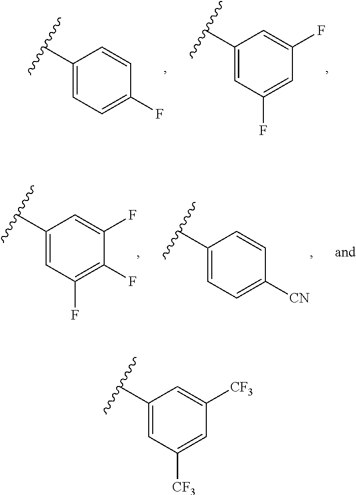

the R⁹—R¹⁰ moiety is selected from the group consisting of:

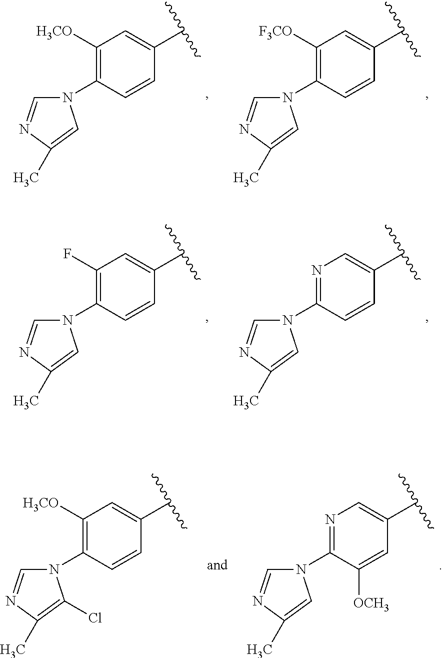

In another embodiment of this invention R⁷ is selected from the group consisting of:

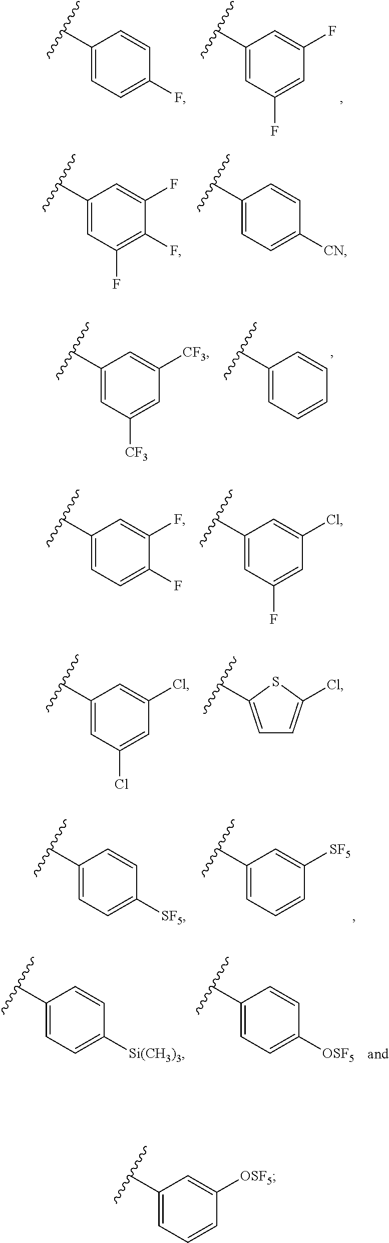

and
the R⁹—R¹⁰— moiety is:

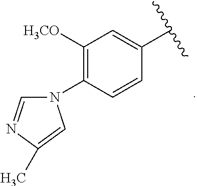

In another embodiment of this invention R⁷ is selected from the group consisting of:

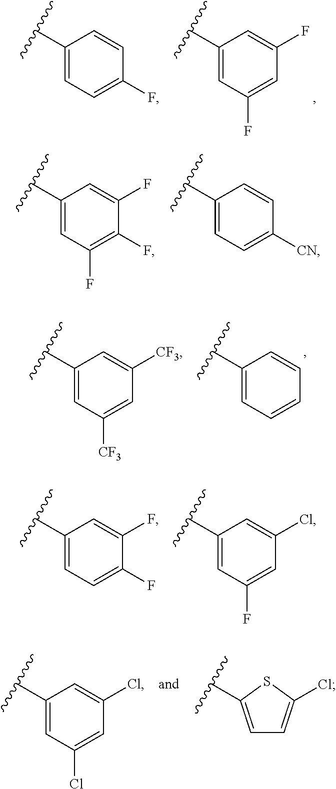

and
the R⁹—R¹⁰— moiety is:

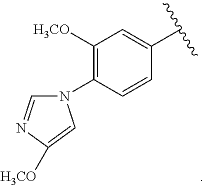

In another embodiment of this invention R⁷ is selected from the group consisting of:

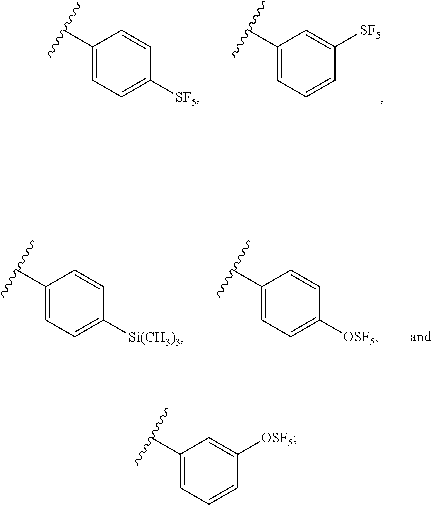

and
the R⁹—R¹⁰— moiety is:

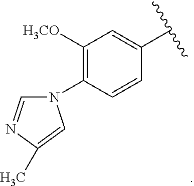

In another embodiment of this invention R⁷ is selected from the group consisting of:

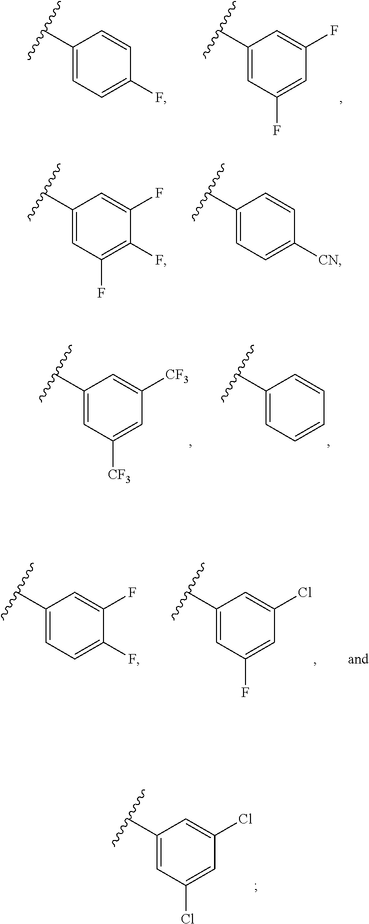

and
the R⁹—R¹⁰— moiety is:

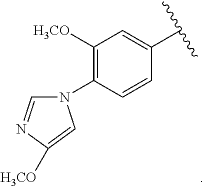

In another embodiment of this invention R⁷ is selected from the group consisting of:

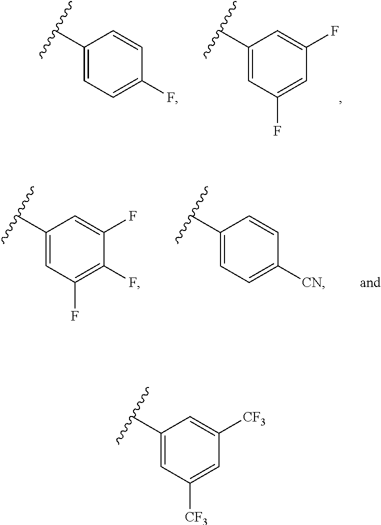

the R⁹—R¹⁰— moiety is:

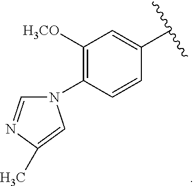

In another embodiment of this invention: (a) R⁶ is alkyl (e.g., methyl), (b) R⁷ is an aryl group, or R⁷ is an aryl group substituted with 1 to 3 independently selected R²¹ groups, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

In another embodiment of this invention: (a) R⁶ is alkyl (e.g., methyl), (b) R⁷ is phenyl, or R⁷ is phenyl substituted with 1 to 3 independently selected R²¹ groups, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

In another embodiment of this invention: (a) R⁶ is alkyl (e.g., methyl), (b) R⁷ is phenyl, or R⁷ is phenyl substituted with 1 to 3 independently selected R²¹ groups, (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

In another embodiment of this invention: (a) R⁶ is alkyl (e.g., methyl), (b) R⁷ is phenyl, or R⁷ is phenyl substituted with 1 to 3 independently selected R²¹ halo groups, (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups.

In another embodiment of this invention: (a) R⁶ is alkyl (e.g., methyl), (b) R⁷ is phenyl, or R⁷ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is alkyl (e.g., methyl), (b) R⁷ is phenyl, or R⁷ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is alkyl (e.g., methyl), (b) R⁷ is phenyl, or R⁷ is phenyl, substituted with 1 to 3 F (i.e., R⁷ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is alkyl (e.g., methyl), (b) R⁷ is phenyl, or R⁷ is phenyl, substituted with 1 to 2 F (i.e., R⁷ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is alkyl (e.g., methyl), (b) R⁷ is phenyl, or R⁷ is phenyl, substituted with 1 F (i.e., R⁷ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is alkyl (e.g., methyl), (b) R⁷ is phenyl substituted with 1 to 3 independently selected R²¹ groups, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

In another embodiment of this invention: (a) R⁶ is alkyl (e.g., methyl), (b) R⁷ is phenyl substituted with 1 to 3 independently selected R²¹ groups, (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

In another embodiment of this invention: (a) R⁶ is alkyl (e.g., methyl), (b) R⁷ is phenyl substituted with 1 to 3 independently selected R²¹ halo groups, (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups.

In another embodiment of this invention: (a) R⁶ is alkyl (e.g., methyl), (b) R⁷ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is alkyl (e.g., methyl), (b) R⁷ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is alkyl (e.g., methyl), (b) R⁷ is phenyl, substituted with 1 to 3 F (i.e., R⁷ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is alkyl (e.g., methyl), (b) R⁷ is phenyl, substituted with 1 to 2 F (i.e., R⁷ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is alkyl (e.g., methyl), (b) R⁷ is phenyl, substituted with 1 F (i.e., R⁷ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is alkyl (e.g., methyl), (b) R⁷ is phenyl, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

In another embodiment of this invention: (a) R⁶ is alkyl (e.g., methyl), (b) R⁷ is phenyl, (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

In another embodiment of this invention: (a) R⁶ is alkyl (e.g., methyl), (b) R⁷ is phenyl, (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups.

In another embodiment of this invention: (a) R⁶ is alkyl (e.g., methyl), (b) R⁷ is phenyl, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is alkyl (e.g., methyl), (b) R⁷ is phenyl, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

In another embodiment of this invention: (a) R⁶ is alkyl (e.g., methyl), (b) R⁷ is phenyl, (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is alkyl (e.g., methyl), (b) R⁷ is phenyl, (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

In another embodiment of this invention: (a) R⁶ is alkyl (e.g., methyl), (b) R⁷ is phenyl, (b) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein R¹⁰ is aryl.

Other embodiments of this invention are directed to any one of the embodiments described in paragraphs (1) to (250) above wherein R¹⁰ is aryl substituted with one or more R²¹ groups.

Other embodiments of this invention are directed to any one of the embodiments described in paragraphs (1) to (250) above wherein R¹⁹ is phenyl.

Other embodiments of this invention are directed to any one of the embodiments described in paragraphs (1) to (250) above wherein R¹⁰ is phenyl substituted with one or more R²¹ groups.

Other embodiments of this invention are directed to any one of the embodiments described in paragraphs (1) to (250) above wherein R¹⁰ is phenyl substituted with one or more R²¹ groups, and each R²¹ group is the same or different —OR¹⁵ group.

Other embodiments of this invention are directed to any one of the embodiments described in paragraphs (1) to (250) above wherein R¹⁰ is phenyl substituted with one or more R²¹ groups, and each R²¹ group is the same or different —OR¹⁵ group, and said R¹⁵ is alkyl, and each alkyl is independently selected.

Other embodiments of this invention are directed to any one of the embodiments described in paragraphs (1) to (250) above wherein R¹⁰ is phenyl substituted with one R²¹ group, and said R²¹ group is —OR¹⁵, and said R¹⁵ is alkyl.

Other embodiments of this invention are directed to any one of the embodiments described in paragraphs (1) to (250) above wherein R¹⁰ is phenyl substituted with one R²¹ group, and said R²¹ group is —OR¹⁵, and said R¹⁵ is alkyl, and said alkyl is methyl.

Other embodiments of this invention are directed to any one of the embodiments described in paragraphs (1) to (250) above wherein R¹⁰ is phenyl substituted with one or more (e.g., one or two, or one) independently selected R²¹ halo groups.

Other embodiments of this invention are directed to any one of the embodiments described in paragraphs (1) to (250) above wherein R¹⁹ is phenyl substituted with one R²¹ group, and said R²¹ group is halo.

Other embodiments of this invention are directed to any one of the embodiments described in paragraphs (1) to (250) above wherein R¹⁰ is phenyl substituted with one R²¹ group, and said R²¹ group is F.

Other embodiments of this invention are directed to any one of the embodiments described in paragraphs (1) to (250) above wherein R¹⁹ is phenyl substituted with one R²¹ group and said R²¹ is an —OR¹⁵ group, and R¹⁵ is an (R¹⁸)ₙalkyl group, and R¹⁸ is halo, and n is 1 to 3, and each halo is independently selected.

Other embodiments of this invention are directed to any one of the embodiments described in paragraphs (1) to (250) above wherein R¹⁹ is phenyl substituted with one R²¹ group and said R²¹ is an —OR¹⁵ group, and R¹⁵ is an (R¹⁸)ₙalkyl group, and R¹⁸ is F, and n is 3.

Other embodiments of this invention are directed to any one of the embodiments described in paragraphs (1) to (250) above wherein R¹⁰ is phenyl substituted with one R²¹ group and said R²¹ is an —OR¹⁵ group, and R¹⁵ is an (R¹⁸)ₙalkyl group, and R¹⁸ is F, and n is 3, and the alkyl is methyl (i.e., the R²¹ substituent is —OCF₃).

Other embodiments of this invention are directed to any one of the embodiments described in paragraphs (1) to (250) above wherein R⁹ is heteroaryl.

Other embodiments of this invention are directed to any one of the embodiments described in paragraphs (1) to (250) above wherein R⁹ is heteroaryl substituted with one or more R²¹ groups.

Other embodiments of this invention are directed to any one of the embodiments described in paragraphs (1) to (250) above wherein R⁹ is heteroaryl substituted with one or more R²¹ groups, and said R²¹ groups are the same or different alkyl.

Other embodiments of this invention are directed to any one of the embodiments described in paragraphs (1) to (250) above wherein R⁹ is heteroaryl substituted with one R²¹ group, and said R²¹ is alkyl.

Other embodiments of this invention are directed to any one of the embodiments described in paragraphs (1) to (250) above wherein R⁹ is heteroaryl substituted with one R²¹ group, and said R²¹ is alkyl, and said alkyl is methyl.

Other embodiments of this invention are directed to any one of the embodiments described in paragraphs (1) to (250) above wherein R⁹ is and said heteroaryl is imidazoyl.

Other embodiments of this invention are directed to any one of the embodiments described in paragraphs (1) to (250) above wherein R⁹ is imidazolyl substituted with one or more R²¹ groups.

Other embodiments of this invention are directed to any one of the embodiments described in paragraphs (1) to (250) above wherein R⁹ is imidazolyl substituted with one or more R²¹ groups, and said R²¹ groups are the same or different alkyl.

Other embodiments of this invention are directed to any one of the embodiments described in paragraphs (1) to (250) above wherein R⁹ is imidazolyl substituted with one R²¹ group, and said R²¹ is alkyl.

Other embodiments of this invention are directed to any one of the embodiments described in paragraphs (1) to (250) above wherein R⁹ is imidazolyl substituted with one R²¹ group, and said R²¹ is alkyl, and said alkyl is methyl.

Other embodiments of this invention are directed to any one of the embodiments described in paragraphs (1) to (250) above wherein R¹⁰ is selected from the group consisting of aryl and aryl substituted with one or more R²¹ groups, and said R⁹ group is selected from the group consisting of heteroaryl and heteroaryl substituted with one or more R²¹ groups, wherein each R²¹ is independently selected.

Other embodiments of this invention are directed to any one of the embodiments described in paragraphs (1) to (250) above wherein R¹⁰ is phenyl substituted with one or more R²¹ groups, and said R⁹ is imidazolyl substituted with one or more R²¹ groups, wherein each R²¹ is independently selected.

Other embodiments of this invention are directed to any one of the embodiments described in paragraphs (1) to (250) above wherein R¹⁰ is phenyl substituted with one R²¹ group, and said R⁹ is imidazolyl substituted with one R²¹ group, wherein each R²¹ is independently selected.

Other embodiments of this invention are directed to any one of the embodiments described in paragraphs (1) to (250) above wherein R¹⁰ is phenyl substituted with one or more independently selected —OR¹⁵ groups, and said R⁹ is imidazolyl substituted with one or more independently selected alkyl groups.

Other embodiments of this invention are directed to any one of the embodiments described in paragraphs (1) to (250) above wherein R¹⁰ is phenyl substituted with one or more independently selected —OR¹⁵ groups, and said R⁹ is imidazolyl substituted with one or more independently selected alkyl groups, and each R¹⁵ is the same or different alkyl group.

Other embodiments of this invention are directed to any one of the embodiments described in paragraphs (1) to (250) above wherein R¹⁰ is phenyl substituted with one —OR¹⁵ group, and said R⁹ is imidazolyl substituted with one alkyl group.

Other embodiments of this invention are directed to any one of the embodiments described in paragraphs (1) to (250) above wherein R¹⁰ is phenyl substituted with one —OR¹⁵ group, and said R⁹ is imidazolyl substituted with one alkyl group, and R¹⁵ is alkyl, and wherein the R¹⁵ alkyl group, and the alkyl group on said imidazolyl are independently selected.

Other embodiments of this invention are directed to any one of the embodiments described in paragraphs (1) to (250) above wherein R¹⁰ is phenyl substituted with one —OR¹⁵ group, and said R⁹ is imidazolyl substituted with one methyl group, and R¹⁵ is methyl, and wherein the R¹⁵ alkyl group, and the alkyl group on said imidazolyl are independently selected.

Other embodiments of this invention are directed to any one of the embodiments described in paragraphs (1) to (250) above wherein R⁹ is:

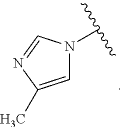

Other embodiments of this invention are directed to any one of the embodiments described in paragraphs (1) to (250) above wherein R¹⁰ is:

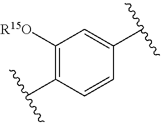

(wherein the —OR¹⁵ is ortho to the carbon to which R⁹ is bound to, i.e., the R⁹—R¹⁰— moiety is:

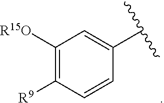

Other embodiments of this invention are directed to any one of the embodiments described in paragraphs (1) to (250) above wherein R¹⁰ is:

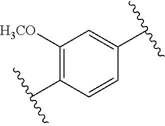

(wherein the —OCH₃ is ortho to the carbon to which R⁹ is bound to, i.e., the R⁹—R¹⁰-moiety is:

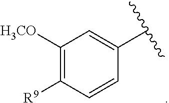

Other embodiments of this invention are directed to any one of the embodiments described in paragraphs (1) to (250) above wherein the R⁹—R¹⁰— moiety is:

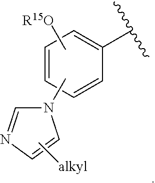

Other embodiments of this invention are directed to any one of the embodiments described in paragraphs (1) to (250) above wherein the R⁹—R¹⁰— moiety is:

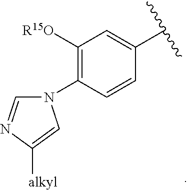

Other embodiments of this invention are directed to any one of the embodiments described in paragraphs (1) to (250) above wherein the R⁹—R¹⁰— moiety is:

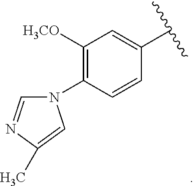

Other embodiments of this invention are directed to any one of the embodiments described in paragraphs (1) to (250) above wherein the R⁹—R¹⁰— moiety is:

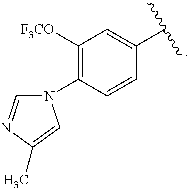

Other embodiments of this invention are directed to any one of the embodiments described in paragraphs (1) to (250) above wherein the R⁹—R¹⁰— moiety is:

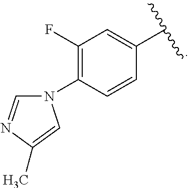

Other embodiments of this invention are directed to any one of the embodiments described in paragraphs (1) to (250) above wherein the R⁹—R¹⁰— moiety is:

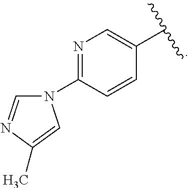

Other embodiments of this invention are directed to any one of the embodiments described in paragraphs (1) to (250) above wherein: (a) R⁶ is alkyl (e.g., methyl), (b) R⁷ is an aryl group, or R⁷ is an aryl group substituted with 1 to 3 independently selected R²¹ groups, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

Other embodiments of this invention are directed to any one of the embodiments described in paragraphs (1) to (250) above wherein: (a) R⁶ is alkyl (e.g., methyl), (b) R⁷ is phenyl, or R⁷ is phenyl substituted with 1 to 3 independently selected R²¹ groups, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

Other embodiments of this invention are directed to any one of the embodiments described in paragraphs (1) to (250) above wherein: (a) R⁶ is alkyl (e.g., methyl), (b) R⁷ is phenyl, or R⁷ is phenyl substituted with 1 to 3 independently selected R²¹ groups, (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

Other embodiments of this invention are directed to any one of the embodiments described in paragraphs (1) to (250) above wherein: (a) R⁶ is alkyl (e.g., methyl), (b) R⁷ is phenyl, or R⁷ is phenyl substituted with 1 to 3 independently selected R²¹ halo groups, (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups.

Other embodiments of this invention are directed to any one of the embodiments described in paragraphs (1) to (250) above wherein: (a) R⁶ is alkyl (e.g., methyl), (b) R⁷ is phenyl, or R⁷ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

Other embodiments of this invention are directed to any one of the embodiments described in paragraphs (1) to (250) above wherein: (a) R⁶ is alkyl (e.g., methyl), (b) R⁷ is phenyl, or R⁷ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

Other embodiments of this invention are directed to any one of the embodiments described in paragraphs (1) to (250) above wherein: (a) R⁶ is alkyl (e.g., methyl), (b) R⁷ is phenyl, or R⁷ is phenyl, substituted with 1 to 3 F (i.e., R⁷ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein: (a) R⁶ is alkyl (e.g., methyl), (b) R⁷ is phenyl, or R⁷ is phenyl, substituted with 1 to 2 F (i.e., R⁷ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein: (a) R⁶ is alkyl (e.g., methyl), (b) R⁷ is phenyl, or R⁷ is phenyl, substituted with 1 F (i.e., R⁷ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein: (a) R⁶ is alkyl (e.g., methyl), (b) R⁷ is phenyl substituted with 1 to 3 independently selected R²¹ groups, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

Other embodiments of this invention are directed to any one of the embodiments described in paragraphs (1) to (250) above wherein: (a) R⁶ is alkyl (e.g., methyl), (b) R⁷ is phenyl substituted with 1 to 3 independently selected R²¹ groups, (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

Other embodiments of this invention are directed to any one of the embodiments described in paragraphs (1) to (250) above wherein: (a) R⁶ is alkyl (e.g., methyl), (b) R⁷ is phenyl substituted with 1 to 3 independently selected R²¹ halo groups, (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups.

Other embodiments of this invention are directed to any one of the embodiments described in paragraphs (1) to (250) above wherein: (a) R⁶ is alkyl (e.g., methyl), (b) R⁷ is phenyl substituted with 1 to 2 independently selected R²¹ halo groups, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

Other embodiments of this invention are directed to any one of the embodiments described in paragraphs (1) to (250) above wherein: (a) R⁶ is alkyl (e.g., methyl), (b) R⁷ is phenyl substituted with 1 R²¹ halo group, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

Other embodiments of this invention are directed to any one of the embodiments described in paragraphs (1) to (250) above wherein: (a) R⁶ is alkyl (e.g., methyl), (b) R⁷ is phenyl, substituted with 1 to 3 F (i.e., R⁷ is phenyl substituted with 1 to 3 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein: (a) R⁶ is alkyl (e.g., methyl), (b) R⁷ is phenyl, substituted with 1 to 2 F (i.e., R⁷ is phenyl substituted with 1 to 2 R²¹ groups, and said R²¹ groups are halo, and said halo is F), (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein: (a) R⁶ is alkyl (e.g., methyl), (b) R⁷ is phenyl, substituted with 1 F (i.e., R⁷ is phenyl substituted with 1 R²¹ group, and said R²¹ group is halo, and said halo is F), (b) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein: (a) R⁶ is alkyl (e.g., methyl), (b) R⁷ is phenyl, (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

Other embodiments of this invention are directed to any one of the embodiments described in paragraphs (1) to (250) above wherein: (a) R⁶ is alkyl (e.g., methyl), (b) R⁷ is phenyl, (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

Other embodiments of this invention are directed to any one of the embodiments described in paragraphs (1) to (250) above wherein: (a) R⁶ is alkyl (e.g., methyl), (b) R⁷ is phenyl, (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups.

Other embodiments of this invention are directed to any one of the embodiments described in paragraphs (1) to (250) above wherein: (a) R⁶ is alkyl (e.g., methyl), (b) R⁷ is phenyl, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

Other embodiments of this invention are directed to any one of the embodiments described in paragraphs (1) to (250) above wherein: (a) R⁶ is alkyl (e.g., methyl), (b) R⁷ is phenyl, (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups.

Other embodiments of this invention are directed to any one of the embodiments described in paragraphs (1) to (250) above wherein: (a) R⁶ is alkyl (e.g., methyl), (b) R⁷ is phenyl, (c) R¹⁹ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein: (a) R⁶ is alkyl (e.g., methyl), (b) R⁷ is phenyl, (c) R¹⁹ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein: (a) R⁶ is alkyl (e.g., methyl), (b) R⁷ is phenyl, (b) R¹⁹ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups.

Other embodiments of this invention are directed to any one of the embodiments described in paragraphs (1) to (250) above wherein ring B is substituted with 1 or two R²¹ groups. In one example each R²¹ group is the same or different alkyl group. In another example each R²¹ is methyl. In another example ring B is substituted with two R²¹ groups. In another example ring B is substituted with two R²¹groups and each group is the same or different alkyl group. In another example ring B is substituted with two R²¹ groups and each group is methyl group. In another example ring B is substituted with one R²¹ group. In another example ring B is substituted with one R²¹ group and said R²¹ group is alkyl. In another example ring B is substituted with one R²¹ group and said R²¹ group is alkyl and said alkyl group is methyl.

In another embodiment of this invention, the compounds of formula (I) are selected from the group consisting of:

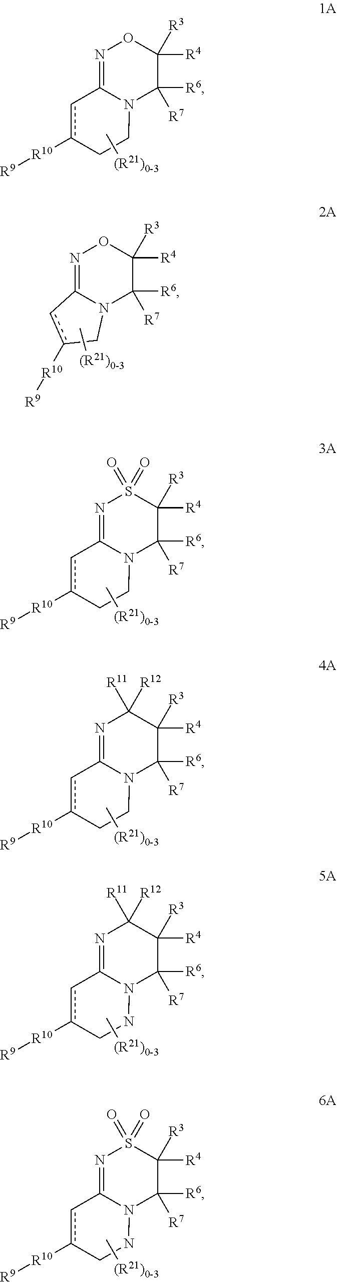

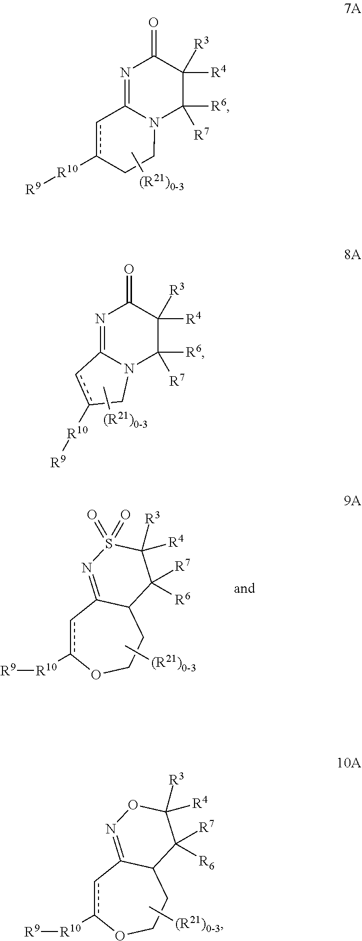

wherein R³, R⁴, R⁶, R⁷, R⁹, R¹⁰, R¹¹, R¹² and R²¹ are as defined for formula (I) or any of the embodiments thereof. In one example of this embodiment there are 0 to 2 R²¹ groups (i.e. there are no R²¹ groups, or there is one R²¹ group, or there are two independently selected R²¹ groups) in ring B. In another example of this embodiment there are two independently selected R²¹ groups in ring B. In another example of this embodiment there is one R²¹ group in ring B. In one example of this embodiment there are 0 to 2 R²¹ groups in ring B wherein said R²¹ groups are the same or different alkyl groups (e.g. methyl). In another example of this embodiment there are two independently selected R²¹ groups in ring B wherein each R²¹ group is the same or different alkyl group (e.g., methyl). In another example of this embodiment there is one R²¹ group and said R²¹ group is alkyl (e.g. methyl). In another example of this embodiment there are no R²¹ groups in ring B.

In another embodiment of this invention, the compounds of formula (I) are selected from the group consisting of:

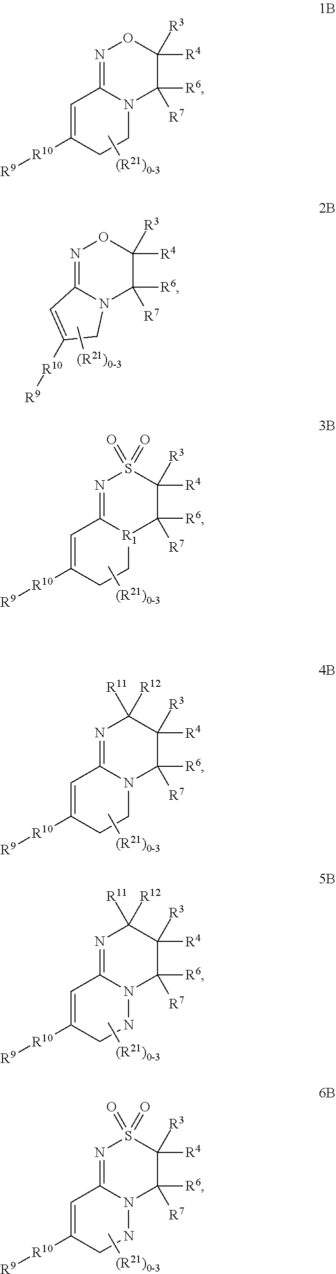

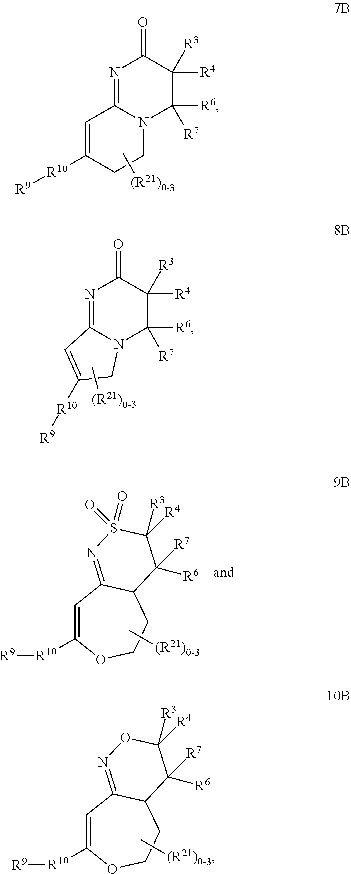

wherein R³, R⁴, R⁶, R⁷, R⁹, R¹⁰, R¹¹, R¹² and R²¹ are as defined for formula (I) or any of the embodiments thereof. In one example of this embodiment there are 0 to 2 R²¹ groups (i.e. there are no R²¹ groups, or there is one R²¹ group, or there are two independently selected R²¹ groups) in ring B. In another example of this embodiment there are two independently selected R²¹ groups in ring B. In another example of this embodiment there is one R²¹ group in ring B. In one example of this embodiment there are 0 to 2 R²¹ groups in ring B wherein said R²¹ groups are the same or different alkyl groups (e.g. methyl). In another example of this embodiment there are two independently selected R²¹ groups in ring B wherein each R²¹ group is the same or different alkyl group (e.g., methyl). In another example of this embodiment there is one R²¹ group and said R²¹ group is alkyl (e.g. methyl). In another example of this embodiment there are no R²¹ groups in ring B.

In another embodiment of this invention, the compounds of formula (I) are selected from the group consisting of:

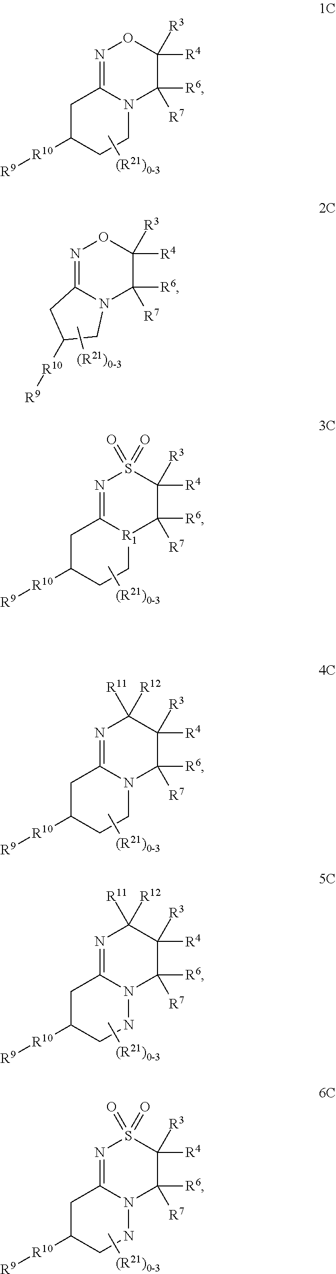

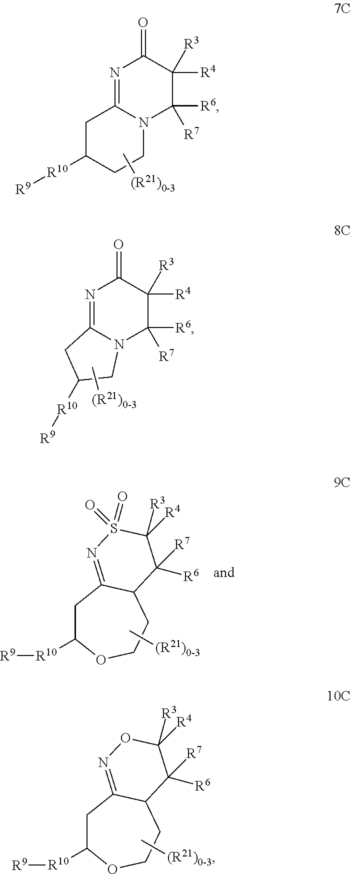

wherein R³, R⁴, R⁶, R⁷, R⁹, R¹⁰, R¹¹, R¹² and R²¹ are as defined for formula (I) or any of the embodiments thereof. In one example of this embodiment there are 0 to 2 R²¹ groups (i.e. there are no R²¹ groups, or there is one R²¹ group, or there are two independently selected R²¹ groups) in ring B. In another example of this embodiment there are two independently selected R²¹ groups in ring B. In another example of this embodiment there is one R²¹ group in ring B. In one example of this embodiment there are 0 to 2 R²¹ groups in ring B wherein said R²¹ groups are the same or different alkyl groups (e.g. methyl). In another example of this embodiment there are two independently selected R²¹ groups in ring B wherein each R²¹ group is the same or different alkyl group (e.g., methyl). In another example of this embodiment there is one R²¹ group and said R²¹ group is alkyl (e.g. methyl). In another example of this embodiment there are no R²¹ groups in ring B.

In another embodiment of this invention, the compounds of formula (I) are selected from the group consisting of:

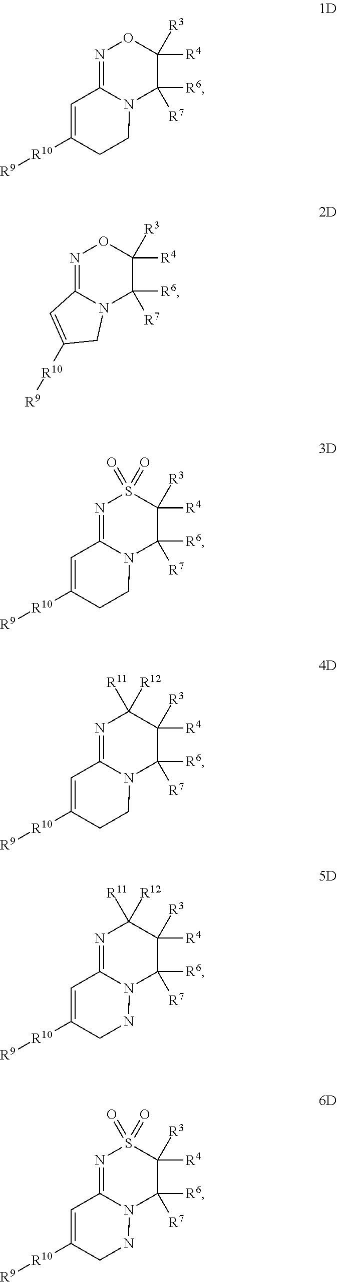

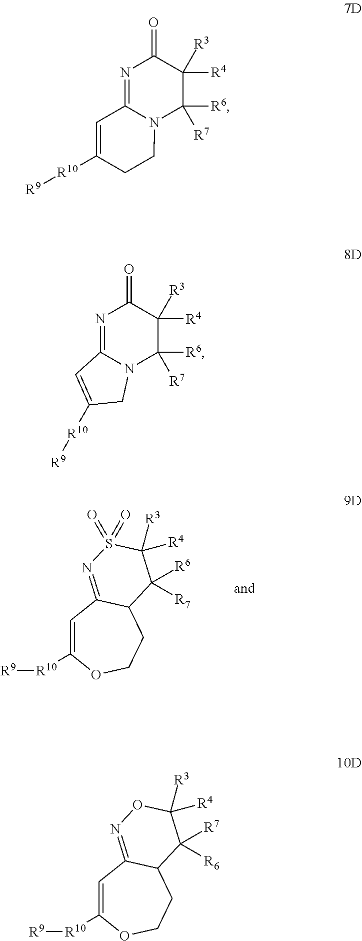

wherein R³, R⁴, R⁶, R⁷, R⁹, R¹⁰, R¹¹, and R¹² are as defined for formula (I) or any of the embodiments thereof.

In another embodiment of this invention, the compounds of formula (I) are selected from the group consisting of:

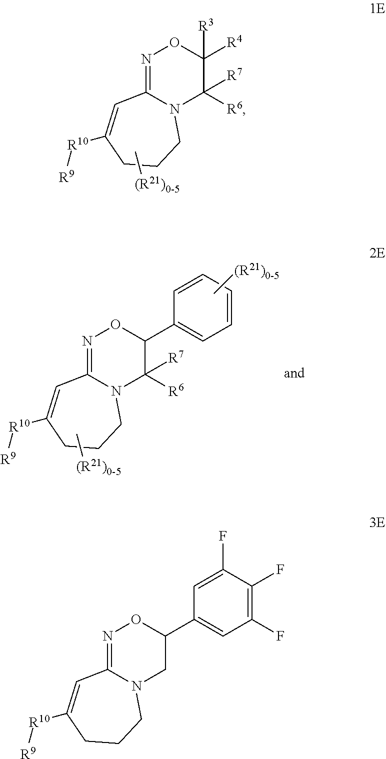

wherein R³, R⁴, R⁶, R⁷, R⁹, R¹⁰, and R²¹ are as defined for formula (I) or any of the embodiments thereof. In one example of this embodiment there are 0 to 2 R²¹ groups (i.e. there are no R²¹ groups, or there is one R²¹ group, or there are two independently selected R²¹ groups) in ring B. In another example of this embodiment there are two independently selected R²¹ groups in ring B. In another example of this embodiment there is one R²¹ group in ring B. In one example of this embodiment there are 0 to 2 R²¹ groups in ring B wherein said R²¹ groups are the same or different alkyl groups (e.g. methyl). In another example of this embodiment there are two independently selected R²¹ groups in ring B wherein each R²¹ group is the same or different alkyl group (e.g., methyl). In another example of this embodiment there is one R²¹ group in Ring B and said R²¹ group is alkyl (e.g. methyl). In another example of this embodiment there are no R²¹ groups in ring B. In another example of this embodiment there are 0 to 3 R²¹ groups in the phenyl ring in 2E. In another example of this embodiment there are 1 to 3 R²¹ groups in the phenyl ring in 2E. In another example of this embodiment there is one R²¹ group in the phenyl ring in 2E. In another example of this embodiment there are two R²¹ groups in the phenyl ring in 2E. In another example of this embodiment there are three R²¹ groups in the phenyl ring in 2E. In another example of this embodiment there are 1 to 3 R²¹ groups in the phenyl ring in 2E, wherein said R²¹ groups are the same or different halo. In another example of this embodiment there are 1 to 3 R²¹ groups in the phenyl ring in 2E, wherein said R²¹ groups are F.

In another embodiment of this invention, the compounds of formula (I) are selected from the group consisting of:

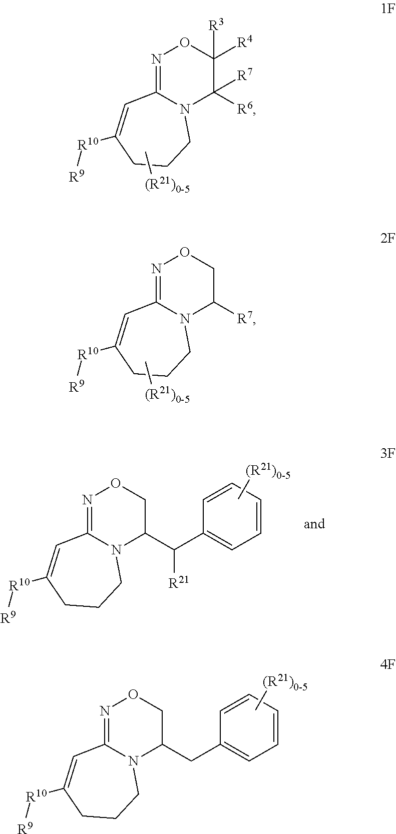

wherein R³, R⁴, R⁶, R⁷, R⁹, R¹⁰, and R²¹ are as defined for formula (I) or any of the embodiments thereof. In one example of this embodiment there are 0 to 2 R²¹ groups (i.e. there are no R²¹ groups, or there is one R²¹ group, or there are two independently selected R²¹ groups) in ring B. In another example of this embodiment there are two independently selected R²¹ groups in ring B. In another example of this embodiment there is one R²¹ group in ring B. In one example of this embodiment there are 0 to 2 R²¹ groups in ring B wherein said R²¹ groups are the same or different alkyl groups (e.g. methyl). In another example of this embodiment there are two independently selected R²¹ groups in ring B wherein each R²¹ group is the same or different alkyl group (e.g., methyl). In another example of this embodiment there is one R²¹ group in Ring B and said R²¹ group is alkyl (e.g. methyl). In another example of this embodiment there are no R²¹ groups in ring B. In another example of this embodiment there are 0 to 3 R²¹ groups in the phenyl ring in 3F. In another example of this embodiment there are 1 to 3 R²¹ groups in the phenyl ring in 3F. In another example of this embodiment there is one R²¹ group in the phenyl ring in 3F. In another example of this embodiment there are two R²¹ groups in the phenyl ring in 3F. In another example of this embodiment there are three R²¹ groups in the phenyl ring in 3F. In another example of this embodiment there are 1 to 3 R²¹ groups in the phenyl ring in 3F, wherein said R²¹ groups are the same or different halo. In another example of this embodiment there are 1 to 3 R²¹ groups in the phenyl ring in 3F, wherein said R²¹ groups are F. In another example of this embodiment there are 0 to 3 R²¹ groups in the phenyl ring in 4F. In another example of this embodiment there are 1 to 3 R²¹ groups in the phenyl ring in 4F. In another example of this embodiment there is one R²¹ group in the phenyl ring in 4F. In another example of this embodiment there are two R²¹ groups in the phenyl ring in 4F. In another example of this embodiment there are three R²¹ groups in the phenyl ring in 4F. In another example of this embodiment there are 1 to 3 R²¹ groups in the phenyl ring in 4F, wherein said R²¹ groups are the same or different halo. In another example of this embodiment there are 1 to 3 R²¹ groups in the phenyl ring in 4F, wherein said R²¹ groups are F.

In another embodiment of this invention, the compounds of formula (I) are selected from the group consisting of:

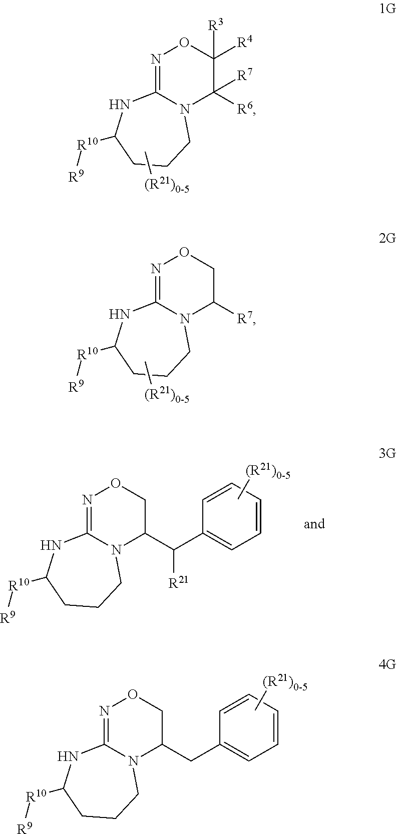

wherein R³, R⁴, R⁶, R⁷, R⁹, R¹⁰, and R²¹ are as defined for formula (I) or any of the embodiments thereof. In one example of this embodiment there are 0 to 2 R²¹ groups (i.e. there are no R²¹ groups, or there is one R²¹ group, or there are two independently selected R²¹ groups) in ring B. In another example of this embodiment there are two independently selected R²¹ groups in ring B. In another example of this embodiment there is one R²¹ group in ring B. In one example of this embodiment there are 0 to 2 R²¹ groups in ring B wherein said R²¹ groups are the same or different alkyl groups (e.g. methyl). In another example of this embodiment there are two independently selected R²¹ groups in ring B wherein each R²¹ group is the same or different alkyl group (e.g., methyl). In another example of this embodiment there is one R²¹ group in Ring B and said R²¹ group is alkyl (e.g. methyl). In another example of this embodiment there are no R²¹ groups in ring B. In another example of this embodiment there are 0 to 3 R²¹ groups in the phenyl ring in 3G. In another example of this embodiment there are 1 to 3 R²¹ groups in the phenyl ring in 3G. In another example of this embodiment there is one R²¹ group in the phenyl ring in 3G. In another example of this embodiment there are two R²¹ groups in the phenyl ring in 3G. In another example of this embodiment there are three R²¹ groups in the phenyl ring in 3G. In another example of this embodiment there are 1 to 3 R²¹ groups in the phenyl ring in 3G, wherein said R²¹ groups are the same or different halo. In another example of this embodiment there are 1 to 3 R²¹ groups in the phenyl ring in 3G, wherein said R²¹ groups are F. In another example of this embodiment there are 0 to 3 R²¹ groups in the phenyl ring in 4G. In another example of this embodiment there are 1 to 3 R²¹ groups in the phenyl ring in 4G. In another example of this embodiment there is one R²¹ group in the phenyl ring in 4G. In another example of this embodiment there are two R²¹ groups in the phenyl ring in 4G. In another example of this embodiment there are three R²¹ groups in the phenyl ring in 4G. In another example of this embodiment there are 1 to 3 R²¹ groups in the phenyl ring in 4G, wherein said R²¹ groups are the same or different halo. In another example of this embodiment there are 1 to 3 R²¹ groups in the phenyl ring in 4G, wherein said R²¹ groups are F.

Representative compounds of the invention include, but are not limited to:

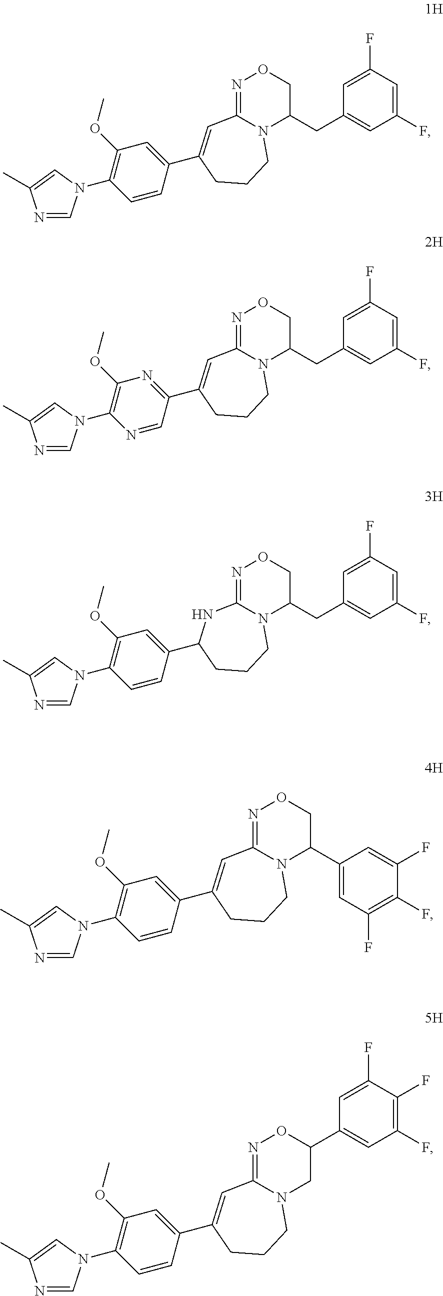

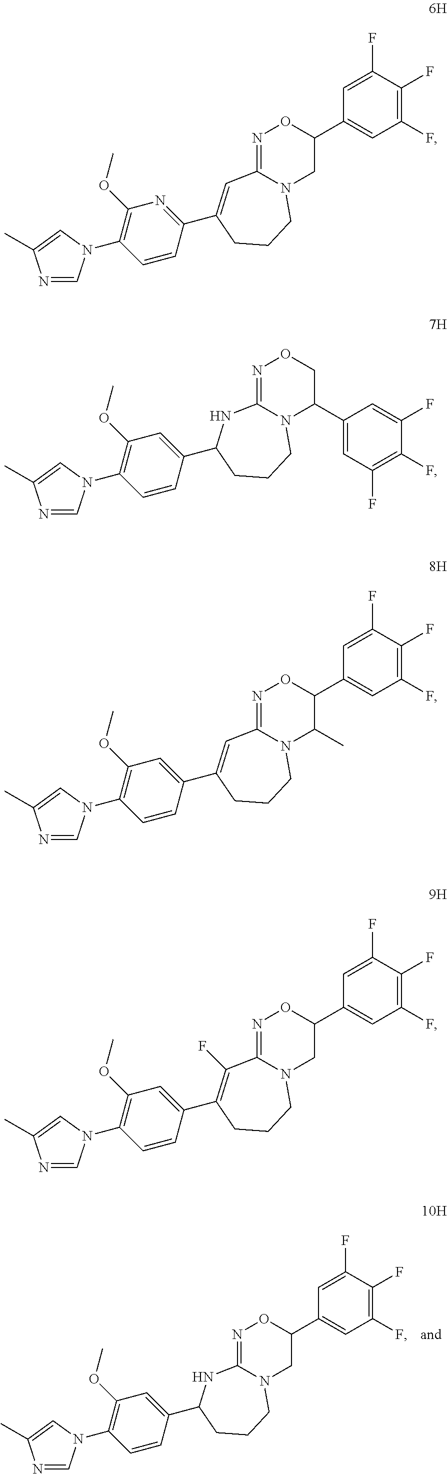

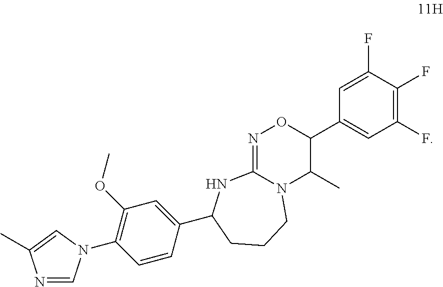

In another embodiment of this invention the compound of formula (I) is a compound of formula 1A.

In another embodiment of this invention the compound of formula (I) is a compound of formula 2A.

In another embodiment of this invention the compound of formula (I) is a compound of formula 3A.

In another embodiment of this invention the compound of formula (I) is a compound of formula 4A.

In another embodiment of this invention the compound of formula (I) is a compound of formula 5A.

In another embodiment of this invention the compound of formula (I) is a compound of formula 6A.

In another embodiment of this invention the compound of formula (I) is a compound of formula 7A.

In another embodiment of this invention the compound of formula (I) is a compound of formula 8A.

In another embodiment of this invention the compound of formula (I) is a compound of formula 9A.

In another embodiment of this invention the compound of formula (I) is a compound of formula 10A.

In another embodiment of this invention the compound of formula (I) is a compound of formula 1B.

In another embodiment of this invention the compound of formula (I) is a compound of formula 2B.

In another embodiment of this invention the compound of formula (I) is a compound of formula 3B.

In another embodiment of this invention the compound of formula (I) is a compound of formula 4B.

In another embodiment of this invention the compound of formula (I) is a compound of formula 5B.

In another embodiment of this invention the compound of formula (I) is a compound of formula 6B.

In another embodiment of this invention the compound of formula (I) is a compound of formula 7B.

In another embodiment of this invention the compound of formula (I) is a compound of formula 8B.

In another embodiment of this invention the compound of formula (I) is a compound of formula 9B.

In another embodiment of this invention the compound of formula (I) is a compound of formula 10B.

In another embodiment of this invention the compound of formula (I) is a compound of formula 1C.

In another embodiment of this invention the compound of formula (I) is a compound of formula 2C.

In another embodiment of this invention the compound of formula (I) is a compound of formula 3C.

In another embodiment of this invention the compound of formula (I) is a compound of formula 4C.

In another embodiment of this invention the compound of formula (I) is a compound of formula 5C.

In another embodiment of this invention the compound of formula (I) is a compound of formula 6C.

In another embodiment of this invention the compound of formula (I) is a compound of formula 7C.

In another embodiment of this invention the compound of formula (I) is a compound of formula 8C.

In another embodiment of this invention the compound of formula (I) is a compound of formula 9C.

In another embodiment of this invention the compound of formula (I) is a compound of formula 10C.

In another embodiment of this invention the compound of formula (I) is a compound of formula 1D.

In another embodiment of this invention the compound of formula (I) is a compound of formula 2D.

In another embodiment of this invention the compound of formula (I) is a compound of formula 3D.

In another embodiment of this invention the compound of formula (I) is a compound of formula 4D.

In another embodiment of this invention the compound of formula (I) is a compound of formula 5D.

In another embodiment of this invention the compound of formula (I) is a compound of formula 6D.

In another embodiment of this invention the compound of formula (I) is a compound of formula 7D.

In another embodiment of this invention the compound of formula (I) is a compound of formula 8D.

In another embodiment of this invention the compound of formula (I) is a compound of formula 9D.

In another embodiment of this invention the compound of formula (I) is a compound of formula 10D.

In another embodiment of this invention the compound of formula (I) is a compound of formula 1E.

In another embodiment of this invention the compound of formula (I) is a compound of formula 2E.

In another embodiment of this invention the compound of formula (I) is a compound of formula 3E.

In another embodiment of this invention the compound of formula (I) is a compound of formula 1F.

In another embodiment of this invention the compound of formula (I) is a compound of formula 2F.

In another embodiment of this invention the compound of formula (I) is a compound of formula 3F.

In another embodiment of this invention the compound of formula (I) is a compound of formula 4F.

In another embodiment of this invention the compound of formula (I) is a compound of formula 1G.

In another embodiment of this invention the compound of formula (I) is a compound of formula 2G.

In another embodiment of this invention the compound of formula (I) is a compound of formula 3G.

In another embodiment of this invention the compound of formula (I) is a compound of formula 4G.

In another embodiment of this invention, the compounds of formula (I) are selected from the group consisting of: 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C, 1D, 2D, 3D, 4D, 5D, 6D, 7D, 8D, 9D and 10D wherein the —R¹⁰—R⁹ moiety is selected from the group consisting of:

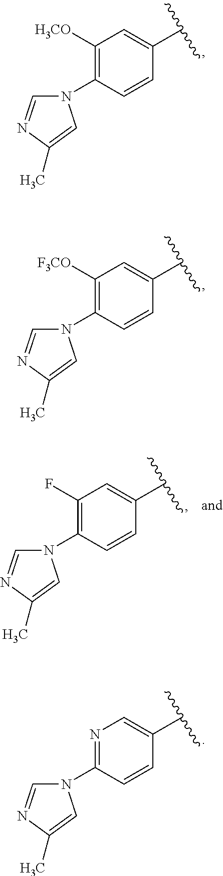

In another embodiment of this invention, the compounds of formula (I) are selected from the group consisting of: 1E, 2E, 3E, 1F, 2F, 3F, 4F, 1G, 2G, 3G, and 4G wherein the —R¹⁰—R⁹ moiety is selected from the group consisting of:

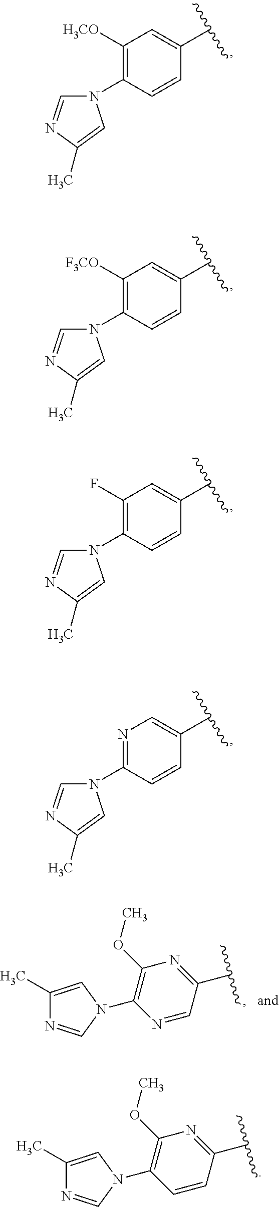

In another embodiment of this invention, the compounds of formula (I) are selected from the group consisting of: 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C, 1D, 2D, 3D, 4D, 5D, 6D, 7D, 8D, 9D and 10D, wherein the —R¹⁰—R⁹ moiety is selected from the group consisting of:

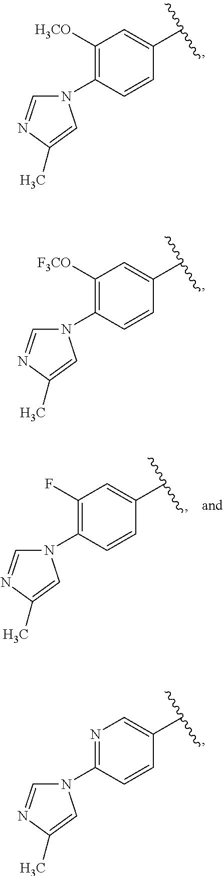

and
wherein R⁶ is alkyl (e.g., methyl), and R⁷ is substituted phenyl (e.g. fluoro substituted phenyl, such as, for example, p-F-phenyl).

In another embodiment of this invention, the compounds of formula (I) are selected from the group consisting of: 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C, 1D, 2D, 3D, 4D, 5D, 6D, 7D, 8D, 9D and 10D, wherein the —R¹⁰—R⁹ moiety is:

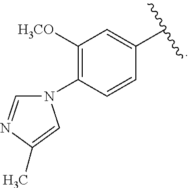

In another embodiment of this invention, the compounds of formula (I) are selected from the group consisting of: 1E, 2E, 3E, 1F, 2F, 3F, 4F, 1G, 2G, 3G, and 4G wherein the —R¹⁰—R⁹ moiety is selected from the group consisting of:

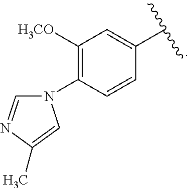

In another embodiment of this invention, the compounds of formula (I) are selected from the group consisting of: 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C, 1D, 2D, 3D, 4D, 5D, 6D, 7D, 8D, 9D and 10D, wherein the —R¹⁰—R⁹ moiety is:

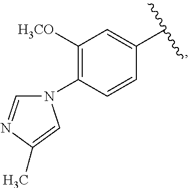

R⁶ is alkyl (e.g., methyl), and R⁷ is substituted phenyl (e.g. fluoro substituted phenyl, such as, for example, p-F-phenyl).

In one embodiment of this invention the compound of formula (I) is 1A, the —R¹⁰—R⁹ moiety is:

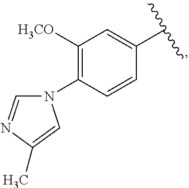

R⁶ is alkyl (e.g., methyl), and R⁷ is substituted phenyl (e.g. fluoro substituted phenyl, such as, for example, p-F-phenyl).

In one embodiment of this invention the compound of formula (I) is 2A, the —R¹⁰—R⁹ moiety is.

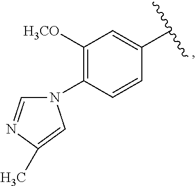

R⁶ is alkyl (e.g., methyl), and R⁷ is substituted phenyl (e.g. fluoro substituted phenyl, such as, for example, p-F-phenyl).

In one embodiment of this invention the compound of formula (I) is 3A, the —R¹⁰—R⁹ moiety is:

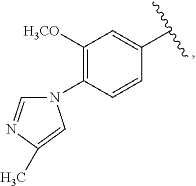

R⁶ is alkyl (e.g., methyl), and R⁷ is substituted phenyl (e.g. fluoro substituted phenyl, such as, for example, p-F-phenyl).

In one embodiment of this invention the compound of formula (I) is 4A, the —R¹⁰—R⁹ moiety is:

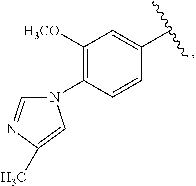

R⁶ is alkyl (e.g., methyl), and R⁷ is substituted phenyl (e.g. fluoro substituted phenyl, such as, for example, p-F-phenyl).

In one embodiment of this invention the compound of formula (I) is 5A, the —R¹⁰—R⁹ moiety is.

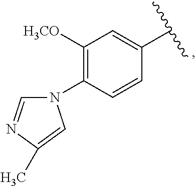

R⁶ is alkyl (e.g., methyl), and R⁷ is substituted phenyl (e.g. fluoro substituted phenyl, such as, for example, p-F-phenyl).

In one embodiment of this invention the compound of formula (I) is 6A, the —R¹⁰—R⁹ moiety is:

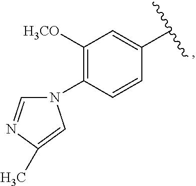

R⁶ is alkyl (e.g., methyl), and R⁷ is substituted phenyl (e.g. fluoro substituted phenyl, such as, for example, p-F-phenyl).

In one embodiment of this invention the compound of formula (I) is 7A, the —R¹⁰—R⁹ moiety is:

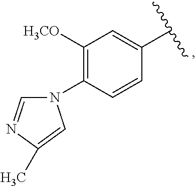

R⁶ is alkyl (e.g., methyl), and R⁷ is substituted phenyl (e.g. fluoro substituted phenyl, such as, for example, p-F-phenyl).

In one embodiment of this invention the compound of formula (I) is 8A, the —R¹⁰—R⁹ moiety is:

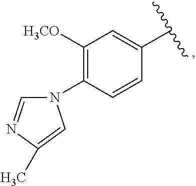

R⁶ is alkyl (e.g., methyl), and R⁷ is substituted phenyl (e.g. fluoro substituted phenyl, such as, for example, p-F-phenyl).

In one embodiment of this invention the compound of formula (I) is 1B, the —R¹⁰—R⁹ moiety is:

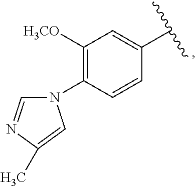

R⁶ is alkyl (e.g., methyl), and R⁷ is substituted phenyl (e.g. fluoro substituted phenyl, such as, for example, p-F-phenyl).

In one embodiment of this invention the compound of formula (I) is 2B, the —R¹⁰—R⁹ moiety is:

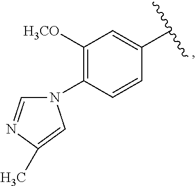

R⁶ is alkyl (e.g., methyl), and R⁷ is substituted phenyl (e.g. fluoro substituted phenyl, such as, for example, p-F-phenyl).

In one embodiment of this invention the compound of formula (I) is 3B, the —R¹⁰—R⁹ moiety is:

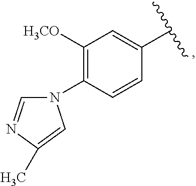

R⁶ is alkyl (e.g., methyl), and R⁷ is substituted phenyl (e.g. fluoro substituted phenyl, such as, for example, p-F-phenyl).

In one embodiment of this invention the compound of formula (I) is 4B, the —R¹⁰—R⁹ moiety is:

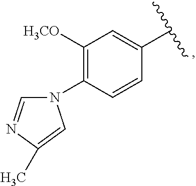

R⁶ is alkyl (e.g., methyl), and R⁷ is substituted phenyl (e.g. fluoro substituted phenyl, such as, for example, p-F-phenyl).

In one embodiment of this invention the compound of formula (I) is 5B, the —R¹⁰—R⁹ moiety is:

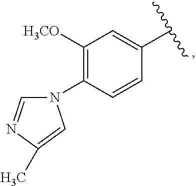

R⁶ is alkyl (e.g., methyl), and R⁷ is substituted phenyl (e.g. fluoro substituted phenyl, such as, for example, p-F-phenyl).

In one embodiment of this invention the compound of formula (I) is 6B, the —R¹⁰—R⁹ moiety is:

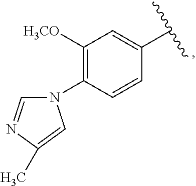

R⁶ is alkyl (e.g., methyl), and R⁷ is substituted phenyl (e.g. fluoro substituted phenyl, such as, for example, p-F-phenyl).

In one embodiment of this invention the compound of formula (I) is 7B, the —R¹⁰—R⁹ moiety is:

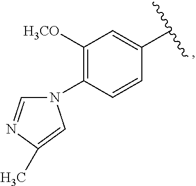

R⁶ is alkyl (e.g., methyl), and R⁷ is substituted phenyl (e.g. fluoro substituted phenyl, such as, for example, p-F-phenyl).

In one embodiment of this invention the compound of formula (I) is 8B, the —R¹⁰—R⁹ moiety is:

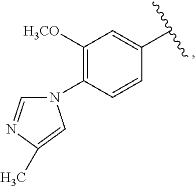

R⁶ is alkyl (e.g., methyl), and R⁷ is substituted phenyl (e.g. fluoro substituted phenyl, such as, for example, p-F-phenyl).

In one embodiment of this invention the compound of formula (I) is 1C, the —R¹⁰—R⁹ moiety is:

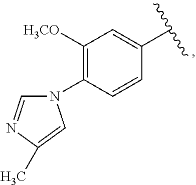

R⁶ is alkyl (e.g., methyl), and R⁷ is substituted phenyl (e.g. fluoro substituted phenyl, such as, for example, p-F-phenyl).

In one embodiment of this invention the compound of formula (I) is 2C, the —R¹⁰—R⁹ moiety is:

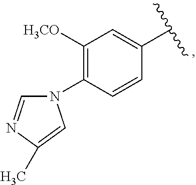

R⁶ is alkyl (e.g., methyl), and R⁷ is substituted phenyl (e.g. fluoro substituted phenyl, such as, for example, p-F-phenyl).

In one embodiment of this invention the compound of formula (I) is 3C, the —R¹⁰—R⁹ moiety is:

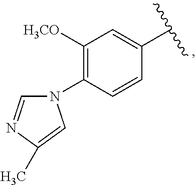

R⁶ is alkyl (e.g., methyl), and R⁷ is substituted phenyl (e.g. fluoro substituted phenyl, such as, for example, p-F-phenyl).

In one embodiment of this invention the compound of formula (I) is 4C, the —R¹⁰—R⁹ moiety is:

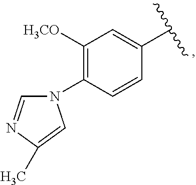

R⁶ is alkyl (e.g., methyl), and R⁷ is substituted phenyl (e.g. fluoro substituted phenyl, such as, for example, p-F-phenyl).

In one embodiment of this invention the compound of formula (I) is 5C, the —R¹⁰—R⁹ moiety is:

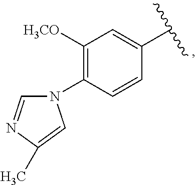

R⁶ is alkyl (e.g., methyl), and R⁷ is substituted phenyl (e.g. fluoro substituted phenyl, such as, for example, p-F-phenyl).

In one embodiment of this invention the compound of formula (I) is 6C, the —R¹⁰—R⁹ moiety is:

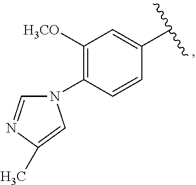

R⁶ is alkyl (e.g., methyl), and R⁷ is substituted phenyl (e.g. fluoro substituted phenyl, such as, for example, p-F-phenyl).

In one embodiment of this invention the compound of formula (I) is 7C, the —R¹⁰—R⁹ moiety is:

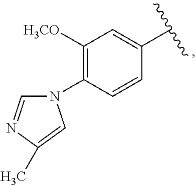

R⁶ is alkyl (e.g., methyl), and R⁷ is substituted phenyl (e.g. fluoro substituted phenyl, such as, for example, p-F-phenyl).

In one embodiment of this invention the compound of formula (I) is 8C, the —R¹⁰—R⁹ moiety is:

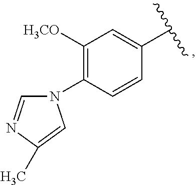

R⁶ is alkyl (e.g., methyl), and R⁷ is substituted phenyl (e.g. fluoro substituted phenyl, such as, for example, p-F-phenyl).

In one embodiment of this invention the compound of formula (I) is 1D, the —R¹⁰—R⁹ moiety is:

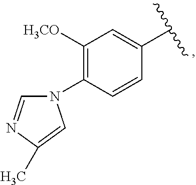

R⁶ is alkyl (e.g., methyl), and R⁷ is substituted phenyl (e.g. fluoro substituted phenyl, such as, for example, p-F-phenyl).

In one embodiment of this invention the compound of formula (I) is 2D, the —R¹⁰—R⁹ moiety is:

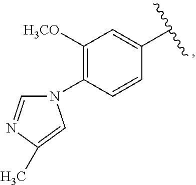

R⁶ is alkyl (e.g., methyl), and R⁷ is substituted phenyl (e.g. fluoro substituted phenyl, such as, for example, p-F-phenyl).

In one embodiment of this invention the compound of formula (I) is 3D, the —R¹⁰—R⁹ moiety is:

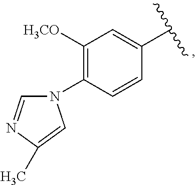

R⁶ is alkyl (e.g., methyl), and R⁷ is substituted phenyl (e.g. fluoro substituted phenyl, such as, for example, p-F-phenyl).

In one embodiment of this invention the compound of formula (I) is 4D, the —R¹⁰—R⁹ moiety is:

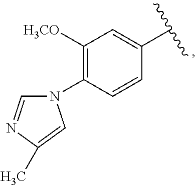

R⁶ is alkyl (e.g., methyl), and R⁷ is substituted phenyl (e.g. fluoro substituted phenyl, such as, for example, p-F-phenyl).

In one embodiment of this invention the compound of formula (I) is 5D, the —R¹⁰—R⁹ moiety is:

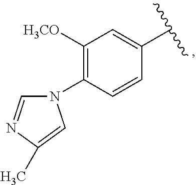

R⁶ is alkyl (e.g., methyl), and R⁷ is substituted phenyl (e.g. fluoro substituted phenyl, such as, for example, p-F-phenyl).

In one embodiment of this invention the compound of formula (I) is 6D, the —R¹⁰—R⁹ moiety is:

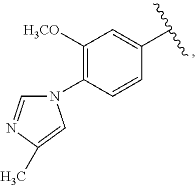

R⁶ is alkyl (e.g., methyl), and R⁷ is substituted phenyl (e.g. fluoro substituted phenyl, such as, for example, p-F-phenyl).

In one embodiment of this invention the compound of formula (I) is 7D, the —R¹⁰—R⁹ moiety is:

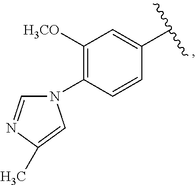

R⁶ is alkyl (e.g., methyl), and R⁷ is substituted phenyl (e.g. fluoro substituted phenyl, such as, for example, p-F-phenyl).

In one embodiment of this invention the compound of formula (I) is 8D, the —R¹⁰—R⁹ moiety is:

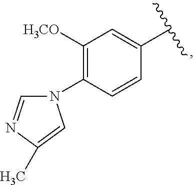

R⁶ is alkyl (e.g., methyl), and R⁷ is substituted phenyl (e.g. fluoro substituted phenyl, such as, for example, p-F-phenyl).

Other embodiments of this invention are directed to compounds 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C, 1D, 2D, 3D, 4D, 5D, 6D, 7D, 8D, 9D and 10D, wherein

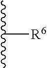

has have the stereochemistry:

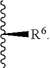

Other embodiments of this invention are directed to compounds 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C, 1D, 2D, 3D, 4D, 5D, 6D, 7D, 8D, 9D and 10D, wherein

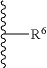

has have the stereochemistry:

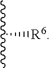

Other embodiments of this invention are directed to compounds 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C wherein the R²¹ groups on ring B have the stereochemistry:

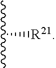

Other embodiments of this invention are directed to compounds 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C wherein the R²¹ groups on ring B have the stereochemistry:

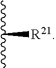

Other embodiments of this invention are directed to compounds 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C wherein each R²¹ group on ring B independently has the stereochemistry selected from the group consisting of:

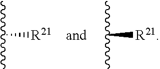

Other embodiments of this invention are directed to compounds 1E, 2E, 3E, 1F, 2F, 3F, 4F, 1G, 2G, 3G, and 4G, wherein

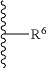

has have the stereochemistry:

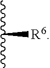

Other embodiments of this invention are directed to compounds 1E, 1F, and 1G wherein

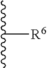

has have the stereochemistry:

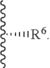

Other embodiments of this invention are directed to compounds 1E, 2E, 1F, 2F, 1G, and 2G wherein the R²¹ groups on ring B have the stereochemistry:

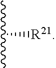

Other embodiments of this invention are directed to compounds 1E, 2E, 1F, 2F, 1G, and 2G wherein the R²¹ groups on ring B have the stereochemistry:

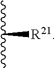

Other embodiments of this invention are directed to compounds 1E, 2E, 1F, 2F, 1G, and 2G wherein each R²¹ group on ring B independently has the stereochemistry selected from the group consisting of:

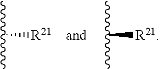

In another embodiment of this invention R²¹ is selected from the group consisting of: alkyl, —OR¹⁵, —C(O)OR¹⁵, —C(O)NR¹⁵R¹⁶, and alkyl substituted with 1 to 5 independently selected R²² groups (e.g., halo, such as, for example, F, Cl, and Br).

In another embodiment of this invention R²¹ is selected from the group consisting of: alkyl, —OR¹⁵, —C(O)OR¹⁵, —C(O)NR¹⁵R¹⁶, and alkyl substituted with 1 to 5 independently selected R²² groups (e.g., halo, such as, for example, F, Cl, and Br, and wherein in one example the alkyl substituted R²¹ group is —CF₃), wherein R¹⁵ and R¹⁶ are independently selected from the group consisting of: H, alkyl, (R¹⁸)ₙ-arylalkyl- (wherein, for example, n is 1, and R¹⁸ is —OR²⁰, and R²⁰ is alkyl (e.g., methyl), cycloalkyl (e.g., cyclobutyl), and (R¹⁸)ₙ-alkyl (e.g, n is 1, R¹⁸ is —OR²⁰, and R²⁰ is alkyl (e.g., methyl).

In another embodiment of this invention R²¹ is selected from the group consisting of: (a) alkyl, —OR¹⁵ (wherein R¹⁵ is alkyl, e.g., methyl and ethyl), (b) —C(O)OR¹⁵ (wherein R¹⁵ is alkyl,e.g., methyl), (c) —C(O)NR¹⁵R¹⁶ (wherein R¹⁵ and R¹⁶ are independently selected from the group consisting of: H, alkyl, (R¹⁸)ₙ-arylalkyl- (wherein, for example, n is 1, and R¹⁸ is —OR²⁰, and R²⁰ is alkyl (e.g., methyl), cycloalkyl (e.g., cyclobutyl), and (R¹⁸)ₙ-alkyl (e.g, n is 1, R¹⁸ is —OR²⁰, and R²⁰ is alkyl (e.g., methyl), and in one example, only one of R¹⁵ and R¹⁶ is H), and (d) alkyl substituted with 1 to 5 independently selected R²² groups (e.g., halo, such as, for example, F, Cl, and Br, and wherein in one example the alkyl substituted R²¹ group is —CF₃).

Other embodiments of this invention are directed to compounds of formula (I) wherein R⁶ or R⁷ is selected from the group consisting of: benzofusedcycloalkyl (i.e., fused benzocycloalkyl), fused benzoheterocycloalkyl, fused heteroarylcycloalkyl, fused heteroarylheterocycloalkyl, and wherein said R⁶ or R⁷ group is optionally substituted with 1-5 independently selected R²¹ groups. In one example, the R²¹ groups are halo (e.g., F).

Examples of the fused ring R⁶ or R⁷ groups include, but are not limited to:

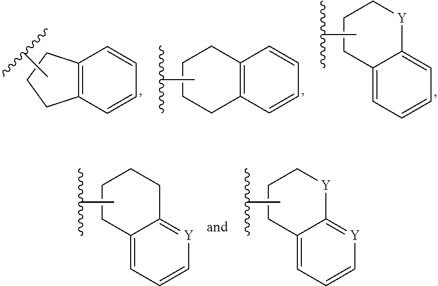

wherein each Y is independently selected from the group consisting of: —O—, —NR¹⁴— and —C(R²¹)_{q}—, (wherein q is 0, 1 or 2 and each R²¹ is independently selected), and wherein R¹⁴ and R²¹ are as defined for formula (I). Examples of these fused ring R⁶ or R⁷ groups include, for example:

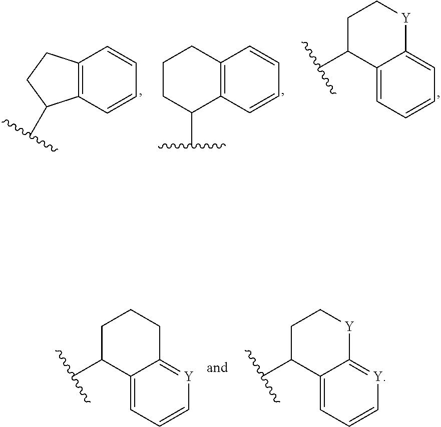

Compounds of formula (I) also include compounds wherein R⁶ or R⁷ is an alkyl group (e.g., methyl or ethyl) substituted with one R²¹ group. Examples of such groups include alkyl (e.g., methyl or ethyl) substituted with the R²¹ moiety aryl (e.g., phenyl or naphthyl). Examples of said R⁶ or R⁷ groups also include alkyl (e.g., methyl or ethyl) substituted with the R²¹ moiety aryl (e.g., phenyl or naphthyl), which in turn is substituted with one or more (e.g., one or two) independently selected R²² groups (e.g., R²² is halo, such as, for example, F).

Examples of the substituted R⁶ or R⁷ alkyl groups include, but are not limited to:

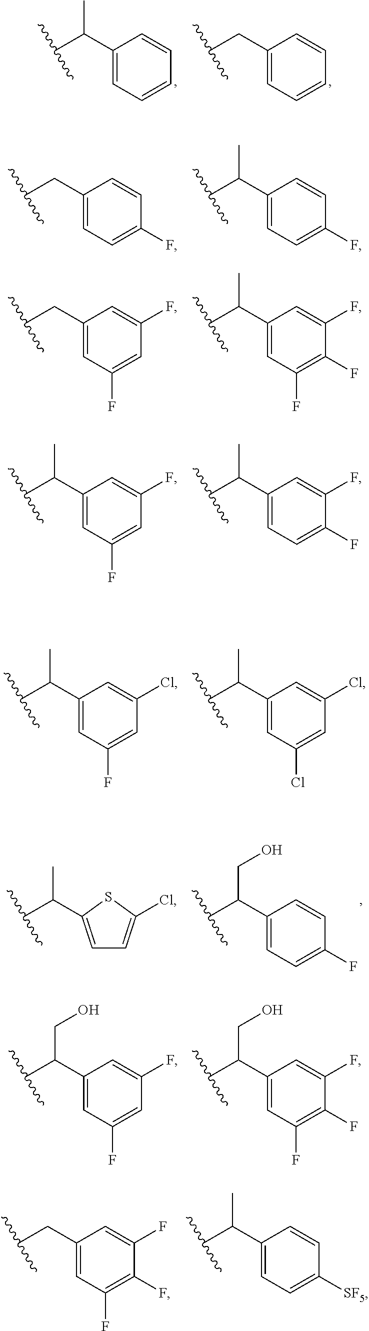

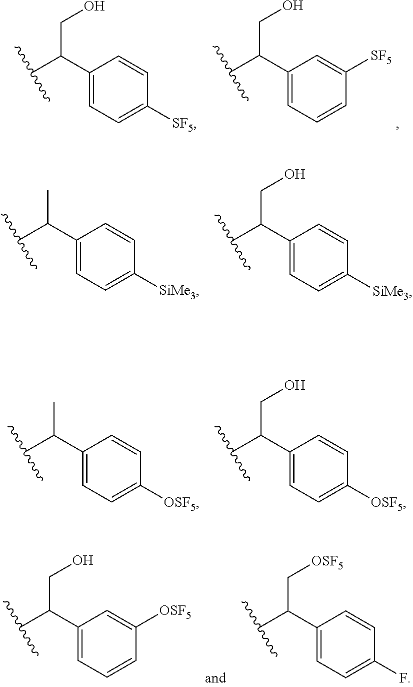

Other embodiments of this invention are directed to compounds of formula (I) wherein R⁶ or R⁷ is a cycloalkyl group (e.g., cyclopropyl or cyclobutyl) substituted with one R²¹ group (e.g., aryl, such as, for example, phenyl), or a cycloalkyl group (e.g., cyclopentyl or cyclohexyl) substituted with one R²¹ group (e.g., aryl, such as, for example, phenyl) which in turn is substituted with one or more (e.g., one or two) independently selected R²² groups (e.g., halo, such as, for example, F). In one example the R²¹ group is bound to the same carbon of the R⁶ or R⁷ group that binds the RR⁶ or R⁷ group to the rest of the molecule.

Examples of the cycloalkyl R⁶ or R⁷ groups include, but are not limited to:

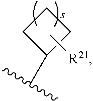

such as, for example,

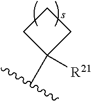

wherein s is 0 (i.e., the ring is cyclopropyl), or 1 (i.e., the ring is cyclobutyl). Examples of these R⁶ or R⁷ groups include, but are not limited to:

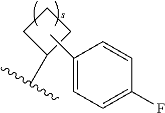

such as, for example,

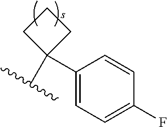

wherein s is 0 (i.e., the ring is cyclopropyl), or 1 (i.e., the ring is cyclobutyl).

Other embodiments of this invention are directed to compounds of formula (I) wherein R⁶ or R⁷ is

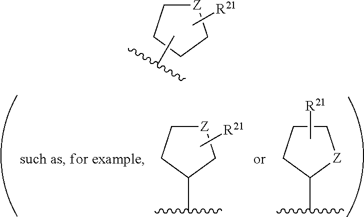

wherein Z is selected from the group consisting of: (1) —O—, (2) —NR¹⁴—, (3) —C(R²¹)_{q}— wherein q is 0, 1 or 2, and each R²¹ is independently selected, (4) —C(R²¹)_{q}—C(R²¹)_{q}— wherein each q is independently 0, 1 or 2 and each R²¹ is independently selected, (5) —(C(R²¹)_{q})_{q}—O—(C(R²¹)_{q})_{q}— wherein each q is independently 0, 1 or 2, and each R²¹ is independently selected, and (6) —(C(R²¹)_{q})_{q}—N(R¹⁴)—(C(R²¹)_{q})_{q}— wherein each q is independently 0, 1 or 2, and each R²¹ is independently selected. Examples of R²¹ include, but are not limited to, aryl (e.g., phenyl) and aryl (e.g., phenyl) substituted with one or more (e.g., one or two, or one) independently selected R²² groups (e.g., halo, such as, for example, F). Examples of this R⁶ or R⁷ include, but are not limited to:

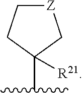

Thus, examples of this R⁶ or R⁷ group include, but are not limited to:

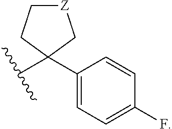

Examples of R⁶ or R⁷ also include, but are not limited to

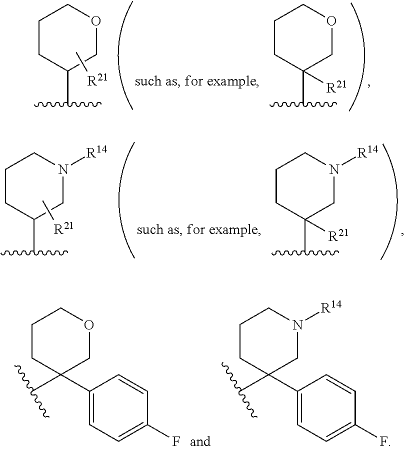

Examples of the R⁶ or R⁷ group

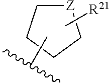

also include, but are not limited to:

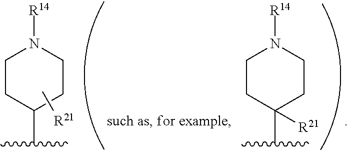

Examples of the R⁶ or R⁷ group

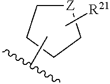

also include, but are not limited to:

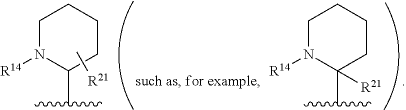

Examples of the R⁶ or R⁷ group

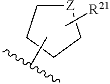

also include, but are not limited to:

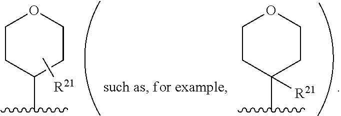

Examples of the R⁶ or R⁷ group

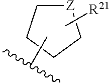

also include, but are not limited to:

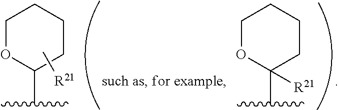

Other embodiments of this invention are directed to compounds of formula (I) wherein R¹⁰ is aryl (e.g., phenyl) or aryl (e.g., phenyl) substituted with one or more (e.g., one or two, or one) R²¹ groups (e.g., −OR¹⁵, wherein, for example, R¹⁵ is alkyl, such as, for example, methyl), and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Thus, examples of the —R¹⁰—R⁹ moiety moiety of the compounds of this invention include, but are not limited to:

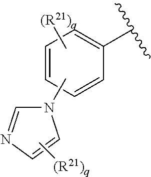

wherein q is 0, 1 or 2, such as, for example,

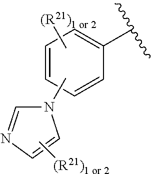

such as, for example,

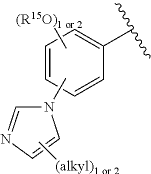

wherein R¹⁵ is alkyl (e.g., methyl), such as, for example,

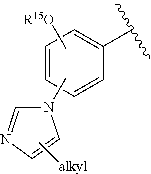

wherein R¹⁵ is alkyl (e.g., methyl), such as, for example,

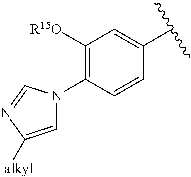

wherein R¹⁵ is alkyl (e.g., methyl), such as, for example,

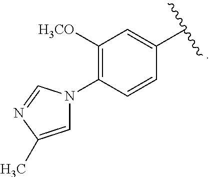

In another embodiment of the compounds of formula (I) R⁶ or R⁷ is benzofusedcycloalkyl.

In another embodiment of the compounds of formula (I) R⁶ or R⁷ is:

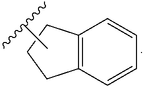

In another embodiment of the compounds of formula (I) R⁶ or R⁷ is:

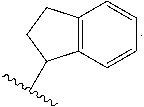

In another embodiment of the compounds of formula (I) R⁶ or R⁷ is:

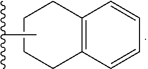

In another embodiment of the compounds of formula (I) R⁶ or R⁷ is:

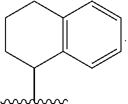

In another embodiment of the compounds of formula (I) R⁶ or R⁷ is alkyl substituted with one R²¹ group.

In another embodiment of the compounds of formula (I) R⁶ or R⁷ is alkyl substituted with one R²¹ group, and said alkyl is

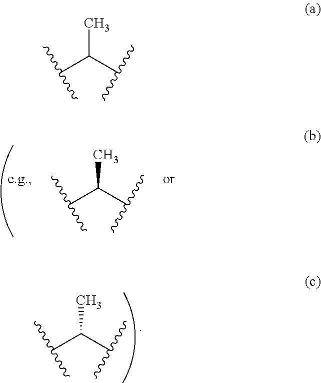

In another embodiment of the compounds of formula (I) R⁶ or R⁷ is alkyl (e.g., (a), (b) or (c) described above) substituted with one R²¹ group wherein said R²¹ group is aryl.

In another embodiment of the compounds of formula (I) R⁶ or R⁷ is alkyl (e.g., (a), (b) or (c) described above) substituted with one R²¹ group wherein said R²¹ group is phenyl.

In another embodiment of the compounds of formula (I) R⁶ or R⁷ is alkyl (e.g., (a), (b) or (c) described above) substituted with one R²¹ group wherein said R²¹ group is naphthyl.

In another embodiment of the compounds of formula (I) R⁶ or R⁷ is alkyl substituted with one R²¹ group, and said R²¹ group is substituted with two independently selected R²² groups.

In another embodiment of the compounds of formula (I) R⁶ or R⁷ is alkyl substituted with one R²¹ group, and said R²¹ group is substituted with one R²² group.

In another embodiment of the compounds of formula (I) R⁶ or R⁷ is alkyl substituted with one R²¹ group, wherein said alkyl group is (a) (e.g., (b) or (c)), as described above, and said R²¹ group is substituted with two independently selected R²² groups.

In another embodiment of the compounds of formula (I) R⁶ or R⁷ is alkyl substituted with one R²¹ group, wherein said alkyl group is (a) (e.g., (b) or (c)), as described above, and said R²¹ group is substituted with one R²² group.

In another embodiment of the compounds of formula (I) R⁶ or R⁷ is alkyl substituted with one R²¹ group, wherein said R²¹ group is aryl, and said R²¹ group is substituted with two independently selected R²² groups.

In another embodiment of the compounds of formula (I) R⁶ or R⁷ is alkyl substituted with one R²¹ group, wherein said R²¹ group is aryl, said alkyl group is (a) (e.g., (b) or (c)), as described above, and said R²¹ group is substituted with two independently selected R²² groups.

In another embodiment of the compounds of formula (I) R⁶ or R⁷ is alkyl substituted with one R²¹ group, wherein said R²¹ group is aryl, wherein said alkyl group is (a) (e.g., (b) or (c)), as described above, and said R²¹ group is substituted with one R²² group.

In another embodiment of the compounds of formula (I) R⁶ or R⁷ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is halo.

In another embodiment of the compounds of formula (I) R⁶ or R⁷ is alkyl substituted with one R²¹ group, wherein said R²¹ group is aryl, wherein said alkyl group is (a) (e.g., (b) or (c)), as described above, and said R²¹ group is substituted with one R²² group and said R²² is halo.

In another embodiment of the compounds of formula (I) R⁶ or R⁷ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is F.

In another embodiment of the compounds of formula (I) R⁶ or R⁷ is alkyl substituted with one R²¹ group, wherein said R²¹ group is aryl, wherein said alkyl group is (a) (e.g., (b) or (c)), as described above, and said R²¹ group is substituted with one R²² group. and said R²² is F.

In another embodiment of the compounds of formula (I) R⁶ or R⁷ is:

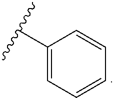

In another embodiment of the compounds of formula (I) R⁶ or R⁷ is:

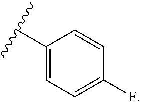

In another embodiment of the compounds of formula (I) R⁶ or R⁷ is:

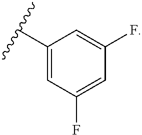

In another embodiment of this invention R⁶ or R⁷ is:

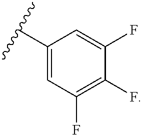

In another embodiment of this invention R⁶ or R⁷ is:

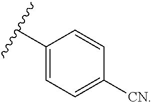

In another embodiment of this invention R⁶ or R⁷ is:

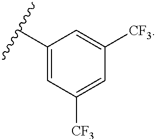

In another embodiment of this invention R⁶ or R⁷ is:

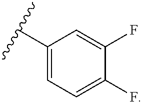

In another embodiment of this invention R⁶ or R⁷ is:

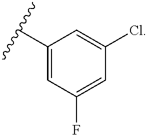

In another embodiment of this invention R⁶ or R⁷ is:

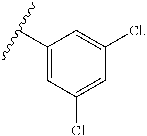

In another embodiment of this invention R⁶ or R⁷ is:

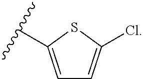

In another embodiment of this invention R⁶ or R⁷ is:

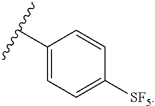

In another embodiment of this invention R⁶ or R⁷ is:

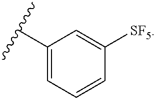

In another embodiment of this invention R⁶ or R⁷ is:

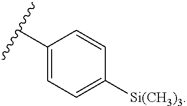

In another embodiment of this invention R⁶ or R⁷ is:

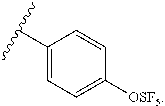

In another embodiment of this invention R⁶ or R⁷ is:

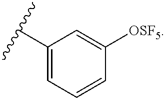

Examples of R²¹ groups include —OR¹⁵ wherein, for example, R¹⁵ is alkyl (such as methyl or ethyl), or R¹⁵ is cycloalkylalkyl (such as, for example, —CH₂-cyclopropyl), or R¹⁵ is -alkyl-(R¹⁸)ₙ (wherein, for example, said R¹⁸ is —OR²⁰, and said R²⁰ is alkyl, and wherein examples of said -alkyl-(R¹⁸)ₙ moiety is —(CH₂)₂OCH₃). Examples of the R²¹ moiety in the embodiments of this invention include, but are not limited to: (a) —OR¹⁵, (b) —OR¹⁵ wherein R¹⁵ is alkyl, (c) —OR¹⁵ wherein R¹⁵ is alkyl and said alkyl is methyl or ethyl, (d) —OR¹⁵ wherein R¹⁵ is cycloalkylalkyl, (e) —OR¹⁵ wherein R¹⁵ is -alkyl-(R¹⁸)ₙ, (f) —OR¹⁵ wherein R¹⁵ is -alkyl-(R¹⁸)ₙ and wherein said R¹⁸ is −OR²⁰, (g) —OR¹⁵ wherein R¹⁵ is -alkyl-(R¹⁸)ₙ and wherein said R¹⁸ is —OR²⁰ and said R²⁰ is alkyl. Examples of the R²¹ moiety include but are not limited to: —OCH₃, —OCH₂CH₃, —O(CH₂)₂OCH₃, and —CH₂-cyclopropyl.

Examples of R²¹ also include —C(O)OR¹⁵ wherein, for example, R¹⁵ is alkyl, such as, for example, methyl).

Examples of R²¹ also include —C(O)NR¹⁵R¹⁶, wherein, for example, one of R¹⁵ or R¹⁸ is H, and the other is selected from the group consisting of: (R¹⁸)ₙ-arylalkyl-, (R¹⁸)ₙ-alkyl-, and cycloalkyl. In one example of this —C(O)NR¹⁵R¹⁶ moiety the R¹⁸ is —OR²⁰, n is 1, R²⁰ is alkyl, said cycloalkyl is cyclobutyl, and said arylalkyl- is benzyl.

Examples of R²¹ also include halo (e.g., Br, Cl or F).

Examples of R²¹ also include arylalkyl, such as, for example, benzyl.

In the embodiments below, Groups A, B and C are defined as follows:
(1) Group A: 1A to 10A, 1B to 10B, 1C to 10C, 1D to 10D, 1E to 3E, 1F to 4F, 1G to 4G, the compounds in numbered paragraphs (1) to (214), the final compound of Example 1, and the final compound of Example 2, the final compound of Example 4, the final compound of Example 5, the final compound of Example 6, the final compound of Example 7, the final compound of Example 8, the final compound of Example 9, and compounds 1H to 11H;
(2) Group B: 1A to 10A, 1B to 10B, 1C to 10C, 1D to 10D, 1E to 3E, 1F to 4F, and 1G to 4G; and
(3) Group C: the compounds in numbered paragraphs (1) to (214).

One embodiment of this invention is directed to a compound of formula (I).

Another embodiment of this invention is directed to a pharmaceutically acceptable salt of a compound of formula (I). And in one example the salt is a salt of a compound selected from the group consisting of Group A. And in another example the salt is a salt of a compound selected from the group consisting of Group B. And in another example the salt is a salt of a compound selected from the group consisting of Group C. And in another example the salt is a salt of a compound selected from the group consisting of Group B. And in another example the salt is a salt of a compound selected from the group consisting of the final compounds of Examples 1, 2, 4 to 9, and compounds 1H to 11H.

Another embodiment of this invention is directed to a pharmaceutically acceptable ester of a compound of formula (I). And in one example the ester is an ester of a compound selected from the group consisting of Group A. And in another example the ester is an ester of a compound selected from the group consisting of Group B. And in another example the ester is an ester of a compound selected from the group consisting of Group C. And in another example the ester is an ester of a compound selected from the group consisting of Group B. And in another example the ester is an ester of a compound selected from the group consisting of the final compounds of Examples 1, 2, 4 to 9, and compounds 1H to 11H.

Another embodiment of this invention is directed to a solvate of a compound of formula (I). And in one example the solvate is a solvate of a compound selected from the group consisting of Group A. And in another example the solvate is a solvate of a compound selected from the group consisting of Group B. And in another example the solvate is a solvate of a compound selected from the group consisting of Group C. And in another example the solvate is a solvate of a compound selected from the group consisting of Group B. And in another example the solvate is a solvate of a compound selected from the group consisting of the final compounds of Examples 1, 2, 4 to 9, and compounds 1H to 11H.

Another embodiment of this invention is directed to a compound of formula (I) in pure and isolated form. And in one example the compound of formula (I) is selected from the group consisting of the final compounds of Examples 1, 2, 4 to 9, and compounds 1H to 11H.

Another embodiment of this invention is directed to a compound of formula (I) in pure form. And in one example the compound of formula (I) is selected from the group consisting of the final compounds of Examples 1, 2, 4 to 9, and compounds 1H to 11H.

Another embodiment of this invention is directed to a compound of formula (I) in isolated form. And in one example the compound of formula (I) is selected from the group consisting of the final compounds of Examples 1, 2, 4 to 9, and compounds 1H to 11H.

Another embodiment of this invention is directed to a compound of formula (I) selected from the group consisting of the final compounds of Examples 1, 2, 4 to 9, and compounds 1H to 11H.

Another embodiment of this invention is directed to pharmaceutically acceptable salt of a compound of formula (I), said compound being selected from the group consisting of the final compounds of Examples 1, 2, 4 to 9, and compounds 1H to 11H

Another embodiment of this invention is directed to the final compound of Example 1.

Another embodiment of this invention is directed to the final compound of Example 2.

Another embodiment of this invention is directed to the final compound of Example 4.

In another embodiment of this invention the compound of formula (I) is the final compound of Example 5.

In another embodiment of this invention the compound of formula (I) is the final compound of Example 6.

In another embodiment of this invention the compound of formula (I) is the final compound of Example 7.

In another embodiment of this invention the compound of formula (I) is the final compound of Example 8.

In another embodiment of this invention the compound of formula (I) is the final compound of Example 9.

In another embodiment of this invention the compound of formula (I) is compound 1H.

In another embodiment of this invention the compound of formula (I) is compound 2H.

In another embodiment of this invention the compound of formula (I) is compound 3H.

In another embodiment of this invention the compound of formula (I) is compound 4H.

In another embodiment of this invention the compound of formula (I) is compound 5H.

In another embodiment of this invention the compound of formula (I) is compound 6H.

In another embodiment of this invention the compound of formula (I) is compound 7H.

In another embodiment of this invention the compound of formula (I) is compound 8H.

In another embodiment of this invention the compound of formula (I) is compound 9H.

In another embodiment of this invention the compound of formula (I) is compound 10H.

In another embodiment of this invention the compound of formula (I) is compound 11H.

Another embodiment of this invention is directed to a pharmaceutically acceptable salt of the final compound of Example 1.

Another embodiment of this invention is directed to a pharmaceutically acceptable salt of the final compound of Example 2.

Another embodiment of this invention is directed to a pharmaceutically acceptable salt of the final compound of Example 4.

Another embodiment of this invention is directed to a pharmaceutically acceptable salt of the final compound of Example 5.

Another embodiment of this invention is directed to a pharmaceutically acceptable salt of the final compound of Example 6.

Another embodiment of this invention is directed to a pharmaceutically acceptable salt of the final compound of Example 7.

Another embodiment of this invention is directed to a pharmaceutically acceptable salt of the final compound of Example 8.

Another embodiment of this invention is directed to a pharmaceutically acceptable salt of the final compound of Example 9.

Another embodiment of this invention is directed to a pharmaceutically acceptable salt of compound 1H.

Another embodiment of this invention is directed to a pharmaceutically acceptable salt of compound 2H.

Another embodiment of this invention is directed to a pharmaceutically acceptable salt of compound 3H.

Another embodiment of this invention is directed to a pharmaceutically acceptable salt of compound 4H.

Another embodiment of this invention is directed to a pharmaceutically acceptable salt of compound 5H.

Another embodiment of this invention is directed to a pharmaceutically acceptable salt of compound 6H.

Another embodiment of this invention is directed to a pharmaceutically acceptable salt of compound 7H.

Another embodiment of this invention is directed to a pharmaceutically acceptable salt of compound 8H.

Another embodiment of this invention is directed to a pharmaceutically acceptable salt of compound 9H.

Another embodiment of this invention is directed to a pharmaceutically acceptable salt of compound 10H.

Another embodiment of this invention is directed to a pharmaceutically acceptable salt of compound 11H.

Another embodiment of this invention is directed to a pharmaceutically acceptable ester of the final compound of Example 1.

Another embodiment of this invention is directed to a pharmaceutically acceptable ester of the final compound of Example 2.

Another embodiment of this invention is directed to a pharmaceutically acceptable ester of the final compound of Example 4.

Another embodiment of this invention is directed to a pharmaceutically acceptable ester of the final compound of Example 5.

Another embodiment of this invention is directed to a pharmaceutically acceptable ester of the final compound of Example 6.

Another embodiment of this invention is directed to a pharmaceutically acceptable ester of the final compound of Example 7.

Another embodiment of this invention is directed to a pharmaceutically acceptable ester of the final compound of Example 8.

Another embodiment of this invention is directed to a pharmaceutically acceptable ester of the final compound of Example 9.

Another embodiment of this invention is directed to a pharmaceutically acceptable ester of compound 1H.

Another embodiment of this invention is directed to a pharmaceutically acceptable ester of compound 2H.

Another embodiment of this invention is directed to a pharmaceutically acceptable ester of compound 3H.

Another embodiment of this invention is directed to a pharmaceutically acceptable ester of compound 4H.

Another embodiment of this invention is directed to a pharmaceutically acceptable ester of compound 5H.

Another embodiment of this invention is directed to a pharmaceutically acceptable ester of compound 6H.

Another embodiment of this invention is directed to a pharmaceutically acceptable ester of compound 7H.

Another embodiment of this invention is directed to a pharmaceutically acceptable ester of compound 8H.

Another embodiment of this invention is directed to a pharmaceutically acceptable ester of compound 9H.

Another embodiment of this invention is directed to a pharmaceutically acceptable ester of compound 10H.

Another embodiment of this invention is directed to a pharmaceutically acceptable ester of compound 11H.

Another embodiment of this invention is directed to a solvate of the final compound of Example 1.

Another embodiment of this invention is directed to a solvate of the final compound of Example 2.

Another embodiment of this invention is directed to a solvate of the final compound of Example 4.

Another embodiment of this invention is directed to a solvate of the final compound of Example 5.

Another embodiment of this invention is directed to a solvate of the final compound of Example 6.

Another embodiment of this invention is directed to a solvate of the final compound of Example 7.

Another embodiment of this invention is directed to a solvate of the final compound of Example 8.

Another embodiment of this invention is directed to a solvate of the final compound of Example 9.

Another embodiment of this invention is directed to a solvate of compound 1H.

Another embodiment of this invention is directed to a solvate of compound 2H.

Another embodiment of this invention is directed to a solvate of compound 3H.

Another embodiment of this invention is directed to a solvate of compound 4H.

Another embodiment of this invention is directed to a solvate of compound 5H.

Another embodiment of this invention is directed to a solvate of compound 6H.

Another embodiment of this invention is directed to a solvate of compound 7H.

Another embodiment of this invention is directed to a solvate of compound 8H.

Another embodiment of this invention is directed to a solvate of compound 9H.

Another embodiment of this invention is directed to a solvate of compound 10H.

Another embodiment of this invention is directed to a solvate of compound 11H.

Another embodiment of this invention is directed to a pharmaceutical composition comprising a therapeutically effective amount of at least one compound of Formula (I), or a pharmaceutically acceptable salt, solvate, or ester thereof, and at least one pharmaceutically acceptable carrier.

Another embodiment of this invention is directed to a pharmaceutical composition comprising an effective amount of one or more (e.g., one) compounds of formula (I) and a pharmaceutically acceptable carrier.

Another embodiment of this invention is directed to a pharmaceutical composition comprising an effective amount of a pharmaceutically acceptable salt of one or more (e.g., one) compounds of formula (I) and a pharmaceutically acceptable carrier.

Another embodiment of this invention is directed to a pharmaceutical composition comprising an effective amount of a pharmaceutically acceptable ester of one or more (e.g., one) compounds of formula (I) and a pharmaceutically acceptable carrier.

Another embodiment of this invention is directed to a pharmaceutical composition comprising an effective amount of a solvate of one or more (e.g., one) compounds of formula (I) and a pharmaceutically acceptable carrier.

Another embodiment of this invention is directed to a pharmaceutical composition comprising an effective amount of one or more (e.g., one) compounds of formula (I), and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising a therapeutically effective amount of at least one compound of Formula (I), or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Another embodiment of this invention is directed to a pharmaceutical composition comprising an effective amount of one or more (e.g., one) compounds of formula (I), and effective amount of one or more BACE inhibitors, and a pharmaceutically acceptable carrier.

Another embodiment of this invention is directed to a pharmaceutical composition comprising an effective amount of one or more (e.g., one) compounds of formula (I), and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds of formula (I), and effective amount of one or more muscarinic antagonists (e.g., m₁ or m₂ antagonists), and a pharmaceutically acceptable carrier.

Another embodiment of this invention is directed to a pharmaceutical composition comprising an effective amount of one or more (e.g., one) compounds of formula I, and effective amount of Exelon (rivastigmine), and a pharmaceutically acceptable carrier.

Another embodiment of this invention is directed to a pharmaceutical composition comprising an effective amount of one or more (e.g., one) compounds of formula I, and effective amount of Cognex (tacrine), and a pharmaceutically acceptable carrier.

Another embodiment of this invention is directed to a pharmaceutical composition comprising an effective amount of one or more (e.g., one) compounds of formula I, and effective amount of a Tau kinase inhibitor, and a pharmaceutically acceptable carrier.

Another embodiment of this invention is directed to a pharmaceutical composition comprising an effective amount of one or more (e.g., one) compounds of formula I, and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of formula I, and effective amount of one anti-Abeta vaccine (active immunization), and a pharmaceutically acceptable carrier.

Another embodiment of this invention is directed to a pharmaceutical composition comprising an effective amount of one or more (e.g., one) compounds of formula I, and effective amount of one or more APP ligands, and a pharmaceutically acceptable carrier.

Another embodiment of this invention is directed to a pharmaceutical composition comprising an effective amount of one or more (e.g., one) compounds of formula I, and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of formula I, and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of formula I, and effective amount of one or more fibrates (for example, clofibrate, Clofibride, Etofibrate, Aluminium Clofibrate), and a pharmaceutically acceptable carrier

Another embodiment of this invention is directed to a pharmaceutical composition comprising an effective amount of one or more (e.g., one) compounds of formula I, and effective amount of one or more LXR agonists, and a pharmaceutically acceptable carrier.

Another embodiment of this invention is directed to a pharmaceutical composition comprising an effective amount of one or more (e.g., one) compounds of formula I, and effective amount of one or more LRP mimics, and a pharmaceutically acceptable carrier.

Another embodiment of this invention is directed to a pharmaceutical composition comprising an effective amount of one or more (e.g., one) compounds of formula I, and effective amount of one or more 5-HT6 receptor antagonists, and a pharmaceutically acceptable carrier.

Another embodiment of this invention is directed to a pharmaceutical composition comprising an effective amount of one or more (e.g., one) compounds of formula I, and effective amount of one or more nicotinic receptor agonists, and a pharmaceutically acceptable carrier.

Another embodiment of this invention is directed to a pharmaceutical composition comprising an effective amount of one or more (e.g., one) compounds of formula I, and effective amount of one or more H3 receptor antagonists, and a pharmaceutically acceptable carrier.

Another embodiment of this invention is directed to a pharmaceutical composition comprising an effective amount of one or more (e.g., one) compounds of formula I, and effective amount of one or more histone deacetylase inhibitors, and a pharmaceutically acceptable carrier.

Another embodiment of this invention is directed to a pharmaceutical composition comprising an effective amount of one or more (e.g., one) compounds of formula I, and effective amount of one or more hsp90 inhibitors, and a pharmaceutically acceptable carrier.

Another embodiment of this invention is directed to a pharmaceutical composition comprising an effective amount of one or more (e.g., one) compounds of formula I, and effective amount of one or more ml muscarinic receptor agonists, and a pharmaceutically acceptable carrier.

Another embodiment of this invention is directed to combinations, i.e., a pharmaceutical composition, comprising a pharmaceutically acceptable carrier, an effective (i.e., therapeutically effective) amount of one or more compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

Another embodiment of this invention is directed to a pharmaceutical composition comprising an effective amount of one or more (e.g., one) compounds of formula I, and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of formula I, and effective amount of one or more one mGluR2/3 antagonists, and a pharmaceutically acceptable carrier.

Another embodiment of this invention is directed to a pharmaceutical composition comprising an effective amount of one or more (e.g., one) compounds of formula I, and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of formula I, and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of formula I, and effective amount of one or more PAI-1 inhibitors, and a pharmaceutically acceptable carrier.

Another embodiment of this invention is directed to a pharmaceutical composition comprising an effective amount of one or more (e.g., one) compounds of formula I, and effective amount of one or more agents that can induce Abeta efflux such as gelsolin, and a pharmaceutically acceptable carrier.

Other embodiments of this invention are directed to any one of the above embodiments directed to pharmaceutical compositions wherein the compound of formula (I) is selected from the group consisting of Group A.

Other embodiments of this invention are directed to any one of the above embodiments directed to pharmaceutical compositions wherein the compound of formula (I) is selected from the group consisting of Group B.

Other embodiments of this invention are directed to any one of the above embodiments directed to pharmaceutical compositions wherein the compound of formula (I) is selected from the group consisting of Group C.

Other embodiments of this invention are directed to any one of the above embodiments directed to pharmaceutical compositions wherein the compound of formula (I) is selected from the group consisting of the final compounds of Examples 1 and 2.

The compounds of formula (I) can be useful as gamma secretase modulators and can be useful in the treatment and prevention of diseases such as, for example, central nervous system disorders (such as Alzheimers disease and Downs Syndrome), mild cognitive impairment, glaucoma, cerebral amyloid angiopathy, stroke, dementia, microgliosis, brain inflammation, and olfactory function loss.

Another embodiment of this invention is directed to a method of treating a central nervous system disorder comprising administering a therapeutically effective amount of at least one compound of Formula (I) to a patient in need of such treatment.

Another embodiment of this invention is directed to a method of treating a central nervous system disorder comprising administering a therapeutically effective amount of a pharmaceutical composition comprising a therapeutically effective amount of at least one compound of Formula (I), or a pharmaceutically acceptable salt, solvate, or ester thereof, and at least one pharmaceutically acceptable carrier. Another embodiment of this invention is directed to a method of treating a central nervous system disorder comprising administering a therapeutically effective amount of a pharmaceutical composition comprising a therapeutically effective amount of at least one compound of Formula (I), or a pharmaceutically acceptable salt, solvate, or ester thereof, and at least one pharmaceutically acceptable carrier, and a therapeutically effective amount of one or more compounds selected from the group consisting of cholinesterase inhibitors, A13 antibody inhibitors, gamma secretase inhibitors and beta secretase inhibitors.

Another embodiment of this invention is directed to a method for modulating (including inhibiting, antagonizing and the like) gamma-secretase comprising administering an effective amount of one or more (e.g., one) compounds of formula (I) to a patient in need of such treatment.

Another embodiment of this invention is directed to a method for modulating (including inhibiting, antagonizing and the like) gamma-secretase, comprising administering an effective amount of a compound of formula (I) to a patient in need of treatment.

Another embodiment of this invention is directed to a method of treating one or more neurodegenerative diseases, comprising administering an effective amount of one or more (e.g., one) compounds of formula (I) to a patient in need of treatment.

Another embodiment of this invention is directed to a method of treating one or more neurodegenerative diseases, comprising administering an effective amount of a compound of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of one or more (e.g., one) compounds of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of a compound of formula (I) to a patient in need of treatment.

Another embodiment of this invention is directed to a method of treating Alzheimer's disease, comprising administering an effective amount of one or more (e.g., one) compounds of formula (I) to a patient in need of treatment.

Another embodiment of this invention is directed to a method of treating Alzheimer's disease, comprising administering an effective amount of a compound of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula (I) to a patient in need of treatment.

Another embodiment of this invention is directed to a method of treating mild cognitive impairment, comprising administering an effective amount of one or more (e.g., one) compounds of formula (I) to a patient in need of treatment.

Another embodiment of this invention is directed to a method of treating glaucoma, comprising administering an effective amount of one or more (e.g., one) compounds of formula (I) to a patient in need of treatment.

Another embodiment of this invention is directed to a method of treating cerebral amyloid angiopathy, comprising administering an effective amount of one or more (e.g., one) compounds of formula (I) to a patient in need of treatment.

Another embodiment of this invention is directed to a method of treating stroke, comprising administering an effective amount of one or more (e.g., one) compounds of formula (I) to a patient in need of treatment.

Another embodiment of this invention is directed to a method of treating dementia, comprising administering an effective amount of one or more (e.g., one) compounds of formula (I) to a patient in need of treatment.

Another embodiment of this invention is directed to a method of treating microgliosis, comprising administering an effective amount of one or more (e.g., one) compounds of formula (I) to a patient in need of treatment.

Another embodiment of this invention is directed to a method of treating brain inflammation, comprising administering an effective amount of one or more (e.g., one) compounds of formula (I) to a patient in need of treatment.

Another embodiment of this invention is directed to a method of treating olfactory function loss, comprising administering an effective amount of one or more (e.g., one) compounds of formula (I) to a patient in need of treatment.

Another embodiment of this invention is directed to a method of treating Downs syndrome, comprising administering an effective amount of one or more (e.g., one) compounds of formula (I) to a patient in need of treatment.

Another embodiment of this invention is directed to a method of treating Downs syndrome, comprising administering an effective amount of a compound of formula (I) to a patient in need of treatment.

Other embodiments of this invention are directed to any one of the above embodiments directed to methods of treating wherein the compound of formula (I) is selected from the group consisting of Group A.

Other embodiments of this invention are directed to any one of the above embodiments directed to methods of treating wherein the compound of formula (I) is selected from the group consisting of Group B.

Other embodiments of this invention are directed to any one of the above embodiments directed to methods of treating wherein the compound of formula (I) is selected from the group consisting of Group C.

Other embodiments of this invention are directed to any one of the above embodiments directed to methods of treating wherein the compound of formula (I) is selected from the group consisting of the final compounds of Examples 1 and 2.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula (I) and the administration of an effective amount of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

Thus, other embodiments of this invention are directed to any one of the methods of treatment, or methods of inhibiting, described herein, wherein an effective amount of the compound of formula (I) is used in combination with an effective amount of one or more other pharmaceutically active ingredients (e.g., drugs). The other pharmaceutically active ingredients (i.e., drugs) are selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to a method of treating Alzheimer's disease, comprising administering an effective amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of a compound of formula (I), in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of one or more (e.g., one) compounds of formula (I), in combination with an effective amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective amount of one or more (e.g., one) compounds of formula (I), in combination with an effective amount of one or more BACE inhibitors.

Another embodiment of this invention is directed to a method of treating Alzheimer's disease, comprising administering an effective amount of one or more compounds of formula I, in combination with an effective amount of Exelon (rivastigmine).

Another embodiment of this invention is directed to a method of treating Alzheimer's disease, comprising administering an effective amount of one or more compounds of formula I, in combination with an effective amount of Cognex (tacrine).

Another embodiment of this invention is directed to a method of treating Alzheimer's disease, comprising administering an effective amount of one or more compounds of formula I, in combination with an effective amount of a Tau kinase inhibitor.

Another embodiment of this invention is directed to a method of treating Alzheimer's disease, comprising administering an effective amount of one or more compounds of formula I, in combination with an effective amount of one or more Tau kinase inhibitor (e.g., GSK3beta inhibitor, cdk5 inhibitor, ERK inhibitor).

This invention also provides a method of treating Alzheimer's disease, comprising administering an effective amount of one or more compounds of formula I, in combination with an effective amount of one anti-Abeta vaccination (active immunization).

Another embodiment of this invention is directed to a method of treating Alzheimer's disease, comprising administering an effective amount of one or more compounds of formula I, in combination with an effective amount of one or more APP ligands.

Another embodiment of this invention is directed to a method of treating Alzheimer's disease, comprising administering an effective amount of one or more compounds of formula I, in combination with an effective amount of one or more agents that upregulate insulin degrading enzyme and/or neprilysin.

Another embodiment of this invention is directed to a method of treating Alzheimer's disease, comprising administering an effective amount of one or more compounds of formula I, in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

This invention also provides a method of treating Alzheimer's disease, comprising administering an effective amount of one or more compounds of formula I, in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds of formula I, in combination with an effective amount of one or more LXR agonists.

Another embodiment of this invention is directed to a method of treating Alzheimer's disease, comprising administering an effective amount of one or more compounds of formula I, in combination with an effective amount of one or more LRP mimics.

Another embodiment of this invention is directed to a method of treating Alzheimer's disease, comprising administering an effective amount of one or more compounds of formula I, in combination with an effective amount of one or more 5-HT6 receptor antagonists.

Another embodiment of this invention is directed to a method of treating Alzheimer's disease, comprising administering an effective amount of one or more compounds of formula I, in combination with an effective amount of one or more nicotinic receptor agonists.

Another embodiment of this invention is directed to a method of treating Alzheimer's disease, comprising administering an effective amount of one or more compounds of formula I, in combination with an effective amount of one or more H3 receptor antagonists.

This invention also provides a method of treating Alzheimer's disease, comprising administering an effective amount of one or more compounds of formula I, in combination with an effective amount of one or more histone deacetylase inhibitors.

Another embodiment of this invention is directed to a method of treating Alzheimer's disease, comprising administering an effective amount of one or more compounds of formula I, in combination with an effective amount of one or more hsp90 inhibitors.

Another embodiment of this invention is directed to a method of treating Alzheimer's disease, comprising administering an effective amount of one or more compounds of formula I, in combination with an effective amount of one or more ml muscarinic receptor agonists.

Another embodiment of this invention is directed to a method of treating Alzheimer's disease, comprising administering an effective amount of one or more compounds of formula I, in combination with an effective amount of one or more 5-HT6 receptor antagonists mGluR1 or mGluR5 positive allosteric modulators or agonists

Another embodiment of this invention is directed to a method of treating Alzheimer's disease, comprising administering an effective amount of one or more compounds of formula I, in combination with an effective amount of one or more mGluR2/3 antagonists.

Another embodiment of this invention is directed to a method of treating Alzheimer's disease, comprising administering an effective amount of one or more compounds of formula I, in combination with an effective amount of one or more anti-inflammatory agents that can reduce neuroinflammation.

Another embodiment of this invention is directed to a method of treating Alzheimer's disease, comprising administering an effective amount of one or more compounds of formula I, in combination with an effective amount of one or more Prostaglandin EP2 receptor antagonists.

Another embodiment of this invention is directed to a method of treating Alzheimer's disease, comprising administering an effective amount of one or more compounds of formula I, in combination with an effective amount of one or more PAI-1 inhibitors.

Another embodiment of this invention is directed to a method of treating Alzheimer's disease, comprising administering an effective amount of one or more compounds of formula I, in combination with an effective amount of one or more agents that can induce Abeta efflux such as gelsolin.

Another embodiment of this invention is directed to a method of treating Downs syndrome, comprising administering an effective amount of one or more (e.g., one) compounds of formula (I), in combination with an effective amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective amount of a compound of formula (I), in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group A.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group B.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of Group C.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of the final compounds of Examples 1 and 2.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase, or (e) mild cognitive impairment, or (f) glaucoma, or (g) cerebral amyloid angiopathy, or (h) stroke, or (i) dementia, or (j) microgliosis, or (k) brain inflammation, or (I) olfactory function loss.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any one of the above embodiments directed to kits wherein the compound of formula (I) is selected from the group consisting of Group A.

Other embodiments of this invention are directed to any one of the above embodiments directed to kits wherein the compound of formula (I) is selected from the group consisting of Group B.

Other embodiments of this invention are directed to any one of the above embodiments directed to kits wherein the compound of formula (I) is selected from the group consisting of Group C.

Other embodiments of this invention are directed to any one of the above embodiments directed to kits wherein the compound of formula (I) is selected from the group consisting of the final compounds of Examples 1, 2 and 4.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ agonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

It is noted that the carbons of formula (I) and other formulas herein may be replaced with 1 to 3 silicon atoms so long as all valency requirements are satisfied.

As used above, and throughout this disclosure, the following terms, unless otherwise indicated, shall be understood to have the following meanings:

“Patient” includes both human and animals.

“Mammal” means humans and other mammalian animals.

“One or more” means that there is at least one and there can be more than one, and examples include 1, 2 or 3, or 1 and 2, or 1.

“At least one” means there is at least one and there can be more than one, and examples include 1, 2 or 3, or 1 and 2, or 1.

“An effective amount” as used to describe the amount of a compound of formula (I) in a pharmaceutical composition, or to describe the amount of a compound of formula (I) used in a method of treatment, or to describe the amount of a pharmaceutical composition used in a method of treatment, or to describe the amount of other pharmaceutic ingredients (i.e., drugs) used in a pharmaceutical compositions or methods of treatment, means a therapeutically effective amount.

“Bn” means benzyl.

“Et” means ethyl.

“i-pr” means isopropyl.

“Me” means methyl.

“Pr” means propyl.

“t-Bu” means tert-butyl.

“TBDMSCl” means tert-butyldimethylsilyl chloride.

“DMAP” means 4-(dimethylamino)pyridine.

“Carbocyclic” means a non-aromatic saturated or unsaturated mono- or multicyclic ring system comprising about 3 to about 10 carbon atoms, preferably about 5 to about 10 carbon atoms. Carbocyclic rings include cycloalkyl rings and cycloalkenyl rings as defined below. Thus, examples of carbocyclic rings include bicyclic rings, such as, for example, norbornyl, adamantly, norbornenyl, and

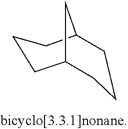

The carbocyclic rings are optionally substituted with one or more independently selected “ring system substituents” as defined below.

“Fused benzocycloalkyl ring” means a phenyl ring fused to a cycloalkyl ring (as cycloalkyl is defined below), such as, for example,

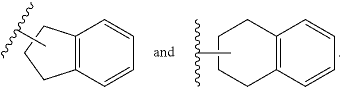

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched.

“Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, ═O, ═N—OY₁, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

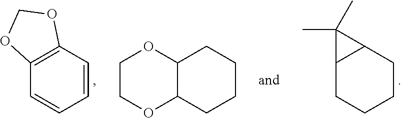

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” or “heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes rings wherein ═O replaces two available hydrogens on the same carbon atom on a ring system (i.e., heterocyclyl includes rings having a carbonyl in the ring). An example of such moiety is pyrrolidone:

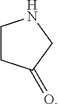

“Heterocyclylalkyl” (or “heterocycloalkylalkyl”) means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” (or “heterocycloalkenyl”) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein ═O replaces two available hydrogens on the same carbon atom on a ring system (i.e., heterocyclyl includes rings having a carbonyl in the ring). An example of such moiety is pyrrolidinone:

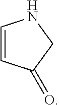

“Heterocyclenylalkyl” (or “heterocycloalkenylalkyl”) means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

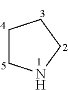

there is no —OH attached directly to carbons marked 2 and 5.

It should also be noted that tautomeric forms such as, for example, the moieties:

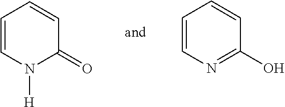

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof,

Compounds of Formula (I), and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl and ¹²³I, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. E.g., those labeled with positron-emitting isotopes like ¹¹C or ¹⁸F can be useful for application in Positron Emission Tomography (PET) and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half lives (T1/2>1 day), can generally be prepared by following procedures analogous to those disclosed in the Schemes and/or in the Examples herein below, by substituting an appropriate isotopically labeled reagent for a non-isotopically labeled reagent.

Polymorphic forms of the compounds of Formula (I), and of the salts, solvates, esters and prodrugs of the compounds of Formula (I), are intended to be included in the present invention.

The compounds according to the invention can have pharmacological properties; in particular, the compounds of Formula (I) can be modulators of gamma secretase (including inhibitors, antagonists and the like).

More specifically, the compounds of Formula (I) can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula (I), or a pharmaceutically acceptable salt, solvate, ester or prodrug of said compound to the mammal.

A preferred dosage is about 0.001 to 500 mg/kg of body weight/day of the compound of Formula (I). An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula (I), or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula I, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula (I), or a pharmaceutically acceptable salt, solvate, ester or prodrug of said compound and a pharmaceutically acceptable carrier, vehicle or diluent.

Yet another aspect of this invention is a kit comprising an amount of at least one compound of Formula (I), or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative schemes and examples which should not be construed to limit the scope of the disclosure. Alternative mechanistic pathways and analogous structures will be apparent to those skilled in the art.

### Example 1

The reaction below is followed to obtain the final compound.

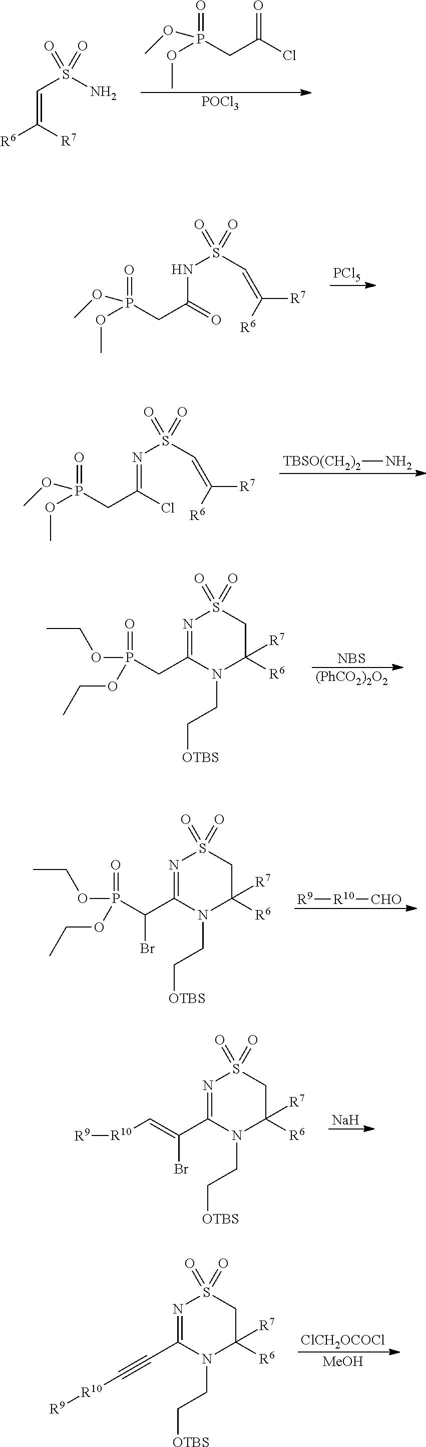

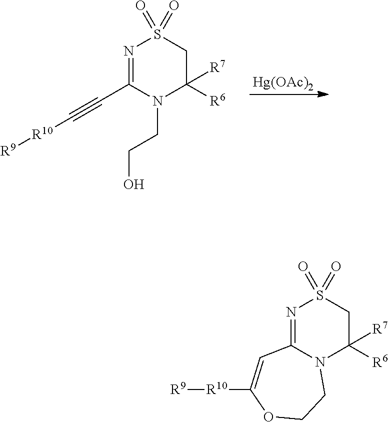

In Example 1, R⁶=Methyl, R⁷=4-F-Phenyl, R⁹=4-(4-Methylimidazol-1-yl), and R¹⁰=3-MeO-Phenyl.

### Example 2

The reaction below is followed to obtain the final compound.

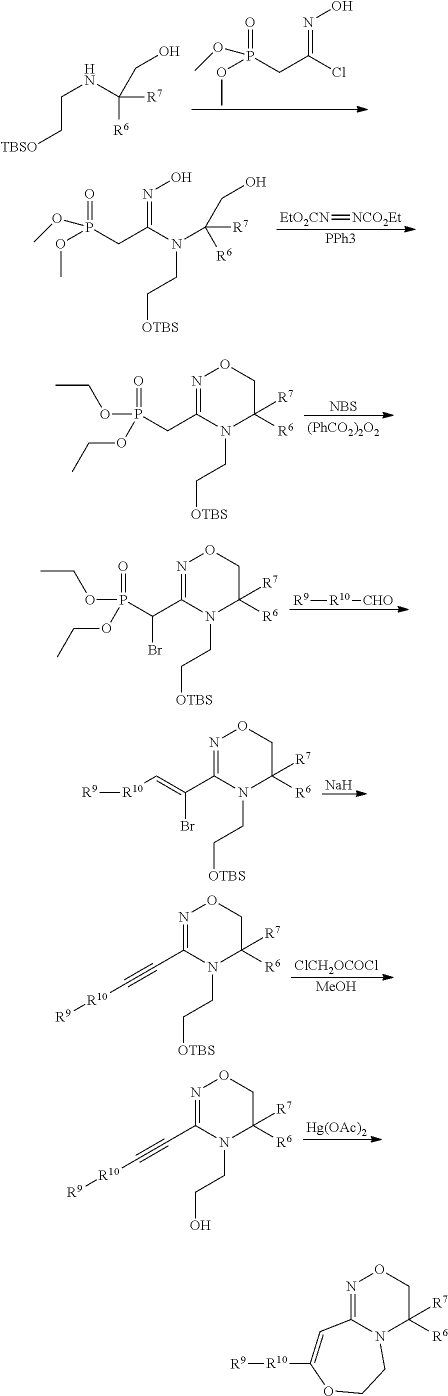

In Example 2, R⁶=Methyl, R⁷=4-F-Phenyl, R⁹=4-(4-Methylimidazol-1-yl), and R¹⁹=3-MeO-Phenyl.

### Example 3

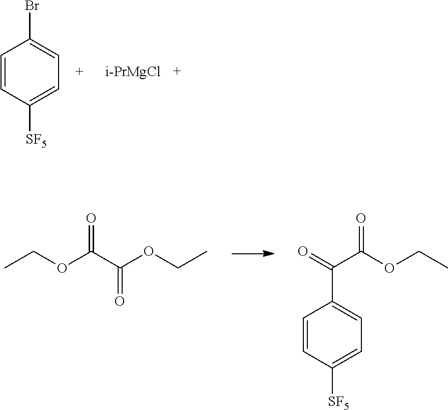

4-bromophenyl sulfur pentabromide (5.2 g, 18.4 mmole) was dissolved in 50 ml THF and the reaction was cooled to −40° C. Isopropylmagnesium chloride lithium chloride complex (1.3M in THF, 14.1 ml) was added and the reaction was stirred for two hours with bath temperature rising to 0° C. This solution was then cannulated to Diethyl oxalate (2.68 g, 18.4 mmole) in 50 ml THF at −78° C. The reaction was stirred for three hours with temperature slowly rising to room temperature. 200 ml water and 200 ml EtOAc were added. The organic layer was washed with water (2×200 ml), dried over Na₂SO₄ and concentrated. The residue was purified by column (EtOAc/hexane from 0/100 to 25/75 in 45 minutes). Yield: 2.0 g. ¹H NMR (CDCl₃ 400 MHz): 8.16 (d, J=8.8 Hz, 2H), 7.91 (d, J=8.8 Hz, 2H), 4.47 (q, J=7.3 Hz, 2H), 1.44 (t, J=7.3 Hz, 3H).

Following a similar procedure the following compound is prepared:

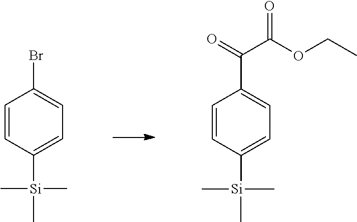

Following these procedures and techniques well known in the art, compounds having —SF₅, —OSF₅ or —Si(R¹⁵)₃ are prepared.

### Example 4

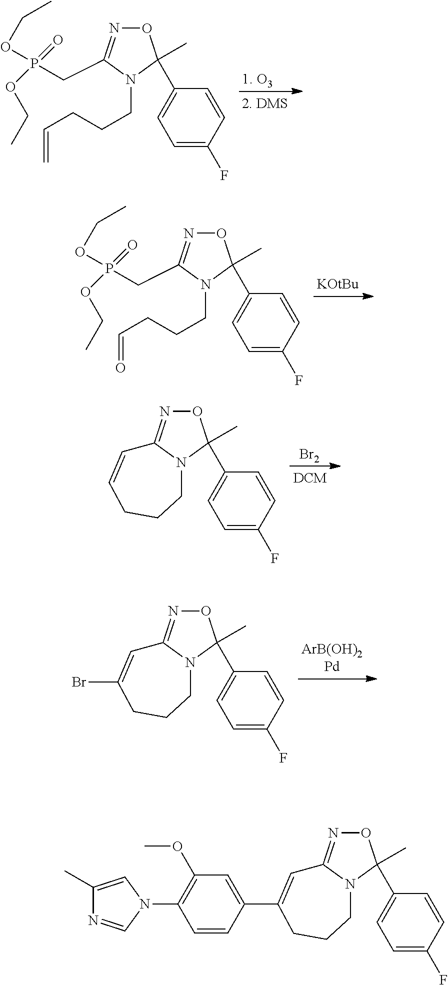

### Example 5

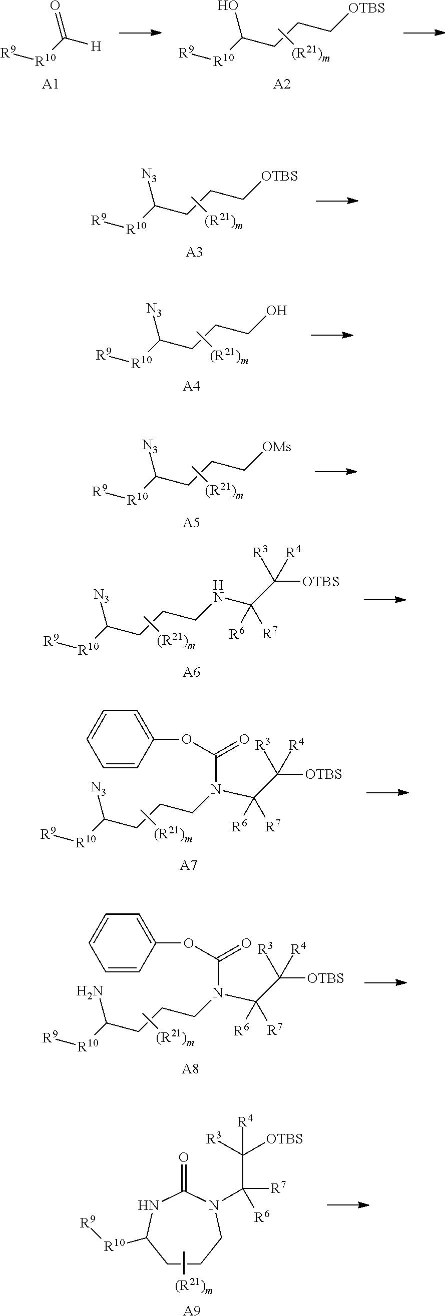

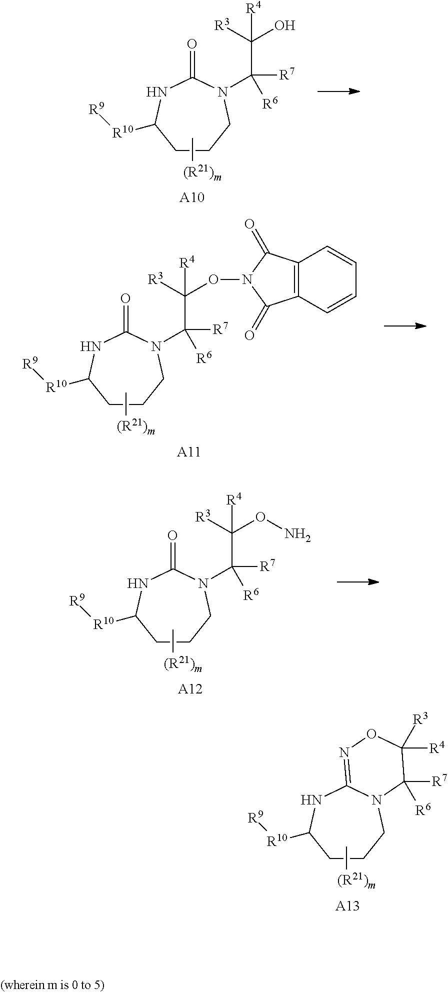

### Example 5

### Step 1

A solution of A1 [R¹⁰=3-MeO-Phenyl; R⁹=4-(4-Methyl-imidazol-1-yl, 9.0 g] in 60 mL anhydrous THF was added dropwise to a stirred suspension of 3-(tert-butyldimethylsiloxy)propylmagnesium bromide (100 mL, 0.5M in THF) at −50° C. under nitrogen atmosphere. The reaction mixture was stirred between −50° C. and room temperature. The reaction mixture was quenched over iced aqueous NH₄Cl, and extracted with EtOAc. The organic phase was dried over anhydrous sodium sulfate. The crude was purified via a flash silica gel column with Hexanes/EtOAc to give product A2 (R¹⁰=3-MeO-Phenyl; R⁹=4-(4-Methyl-imidazol-1-yl; m=0).

### Example 5

### Step 2

DBU (701.5 mg) was added dropwise to a stirred solution of A2 (R¹⁰=3-MeO-Phenyl; R⁹=4-(4-Methyl-imidazol-1-yl; m=0, 1.5 g) and diphenylphosphoryl azide in 8 mL of anhydrous THF at 0° C. under nitrogen atmosphere. Once the addition was completed, the ice-bath was removed and reaction mixture was allowed to continue stirring at room temperature for 2 days. The reaction mixture was then worked up under aqueous condition and extracted with DCM. The crude was purified via a flash silica gel column with Hexanes/EtOAc to afford product A3 (R¹⁰=3-MeO-Phenyl; R⁹=4-(4-Methyl-imidazol-1-yl; m=0).

### Example 5

### Step 3

A solution of 4N hydrochloric acid in 1,4-dioxane (20 mL) and A3 (R¹³ MeO-Phenyl; R⁹=4-(4-Methyl-imidazol-1-yl; m=0, 1.0 g) was stirred at room temperature overnight. The solvent was removed in vacuo to obtain a residue that was then precipitated in DCM and ether solution to obtain a white, amorphous solid, which was dried in vacuo to afford A4 (R¹⁰=3-MeO-Phenyl; R⁹=4-(4-Methyl-imidazol-1-yl; m=0).

### Example 5

### Step 4

Methanesulfonyl chloride (171 mg) was added dropwise to a solution of A4 (R¹³=3-MeO-Phenyl; R⁹=4-(4-Methyl-imidazol-1-yl; m=0, 300 mg) and triethylamine (403 mg) in 10 mL of anhydrous DCM at 0° C. under nitrogen atmosphere. The reaction mixture was stirred between 0° C. and 10° C. over 2 hrs, and quenched over iced brine. The mixture was extracted with DCM. The organic phase was dried over anhydrous sodium sulfate to give crude A5 (R¹⁰=3-MeO-Phenyl; R⁹=4-(4-Methyl-imidazol-1-yl; m=0) which was used in next reaction without purification.

### Example 5

### Step 5

A suspension of A5 (R¹⁰=3-MeO-Phenyl; R⁹=4-(4-Methyl-imidazol-1-yl; m=0, 1 equiv), 2-(tert-butyldimethylsilyloxy)-1-(3,4,5-trifluorophenyl)ethanamine (5 equiv), and potassium iodide (0.2 equiv) in acetonitrile will be heated in a microwave reactor at 120° C. to afford A6 (R¹⁰=3-MeO-Phenyl; R⁹=4-(4-Methyl-imidazol-1-yl; R³, R⁴, and R⁷═H; m=0; R⁶=3,4,5-trifluorophenyl).

### Example 5

### Step 6

Phenyl chloroformate (1.1 equiv) in anhydrous DCM will be added dropwise to a stirred solution of A6 (R¹⁰=3-MeO-Phenyl; R⁹=4-(4-Methyl-imidazol-1-yl; R³, R⁴ and R⁷; m=0; R⁶=3,4,5-trifluorophenyl, 1 equiv) and triethylamine (1.8 equiv) in anhydrous DCM at 0° C. under nitrogen atmosphere. The reaction will be allowed to proceed at room temperature to afford A7 (R¹⁰=3-MeO-Phenyl; R⁹=4-(4-Methyl-imidazol-1-yl; R³, R⁴ and R⁷═H; m=0; R⁶=3,4,5-trifluorophenyl).

### Example 5

### Step 7

A7 (R¹⁰=3-MeO-Phenyl; R⁹=4-(4-Methyl-imidazol-1-yl; R³, R⁴ and R⁷═H; m=0; R⁶=3,4,5-trifluorophenyl) will be reduced by hydrogenation in EtOAc catalyzed by Pd/C to afford A8 (R¹⁰=3-MeO-Phenyl; R⁹=4-(4-Methyl-imidazol-1-yl; R³, R⁴ and R⁷═H; m=0; R⁶=3,4,5-trifluorophenyl).

### Example 5

### Step 8

A8 (R¹⁰=3-MeO-Phenyl; R⁹=4-(4-Methyl-imidazol-1-yl; R³, R⁴ and R⁷═H; m=0; R⁶=3,4,5-trifluorophenyl) will be treated with methylmagnesium bromide (3M in ether, 1.1 equiv) in DCM at 45° C. overnight to afford A9 (R¹⁰=3-MeO-Phenyl; R⁹=4-(4-Methyl-imidazol-1-yl; R³, R⁴ and R⁷═H; m=0; R⁶=3,4,5-trifluorophenyl).

### Example 5

### Step 9

A9 (R¹⁰=3-MeO-Phenyl; R⁹=4-(4-Methyl-imidazol-1-yl; R³, R⁴ and R⁷═H; m=0; R⁶ 3,4,5-trifluorophenyl) will be treated with 4N HCl in dioxane to afford A10 (R¹⁶=3-MeO-Phenyl; R⁹=4-(4-Methyl-imidazol-1-yl; R³, R⁴ and R⁷═H; m=0; R⁶=3,4,5-trifluorophenyl).

### Example 5

### Step 10

A10 (R¹⁰=3-MeO-Phenyl; R⁹=4-(4-Methyl-imidazol-1-yl; R³, R⁴ and R⁷═H; m=0; R⁶=3,4,5-trifluorophenyl, 1 equiv) will be treated with N-hydroxyphthalimide (1.8 equiv), Ph₃P (2 equiv), and DIAD (2 equiv) in THF at room temperature to afford All (R¹⁰=3-MeO-Phenyl; R⁹=4-(4-Methyl-imidazol-1-yl; R³, R⁴ and R⁷═H; m=0; R⁶=3,4,5-trifluorophenyl).

### Example 5

### Step 11

A solution of A11 (R¹⁰=3-MeO-Phenyl; R⁹=4-(4-Methyl-imidazol-1-yl; R³, R⁴ and R⁷═H; m=0; R⁶=3,4,5-trifluorophenyl, 1 equiv) in EtOH will be treated with 1M hydrazine in THF (1.5 equiv) at room temperature for 6 hrs to afford A12 (R¹⁰=3-MeO-Phenyl; R⁹=4-(4-Methyl-imidazol-1-yl; R³, R⁴ and R⁷=0; R⁶=3,4,5-trifluorophenyl).

### Example 5

### Step 12

A solution of A12 (R¹⁹=3-MeO-Phenyl; R⁹=4-(4-Methyl-imidazol-1-yl; R³, R⁴ and R⁷═H; m=0; R⁶=3,4,5-trifluorophenyl, 1 equiv) in EtOH will be treated with 85% H₃PO₄ (3 equiv) at 90° C. overnight to afford A13 (R¹⁹=3-MeO-Phenyl; R⁹=4-(4-Methyl-imidazol-1-yl; R³, R⁴ and R⁷═H; m=0; R⁶=3,4,5-trifluorophenyl).

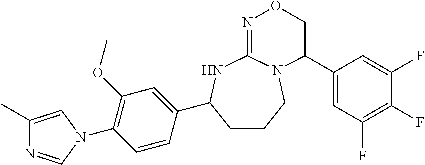

### Example 6

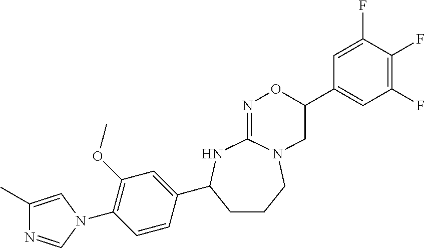

This compound will be made using a method similar to Example 5.

### Example 7

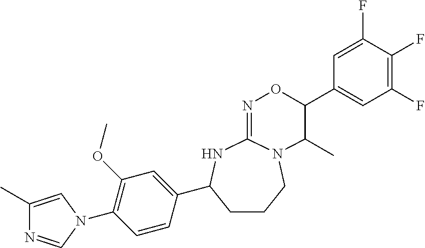

This compound will be made using a method similar to Example 5.

### Example 8

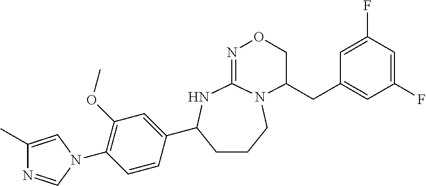

This compound will be made using a method similar to Example 5.

### Example 9

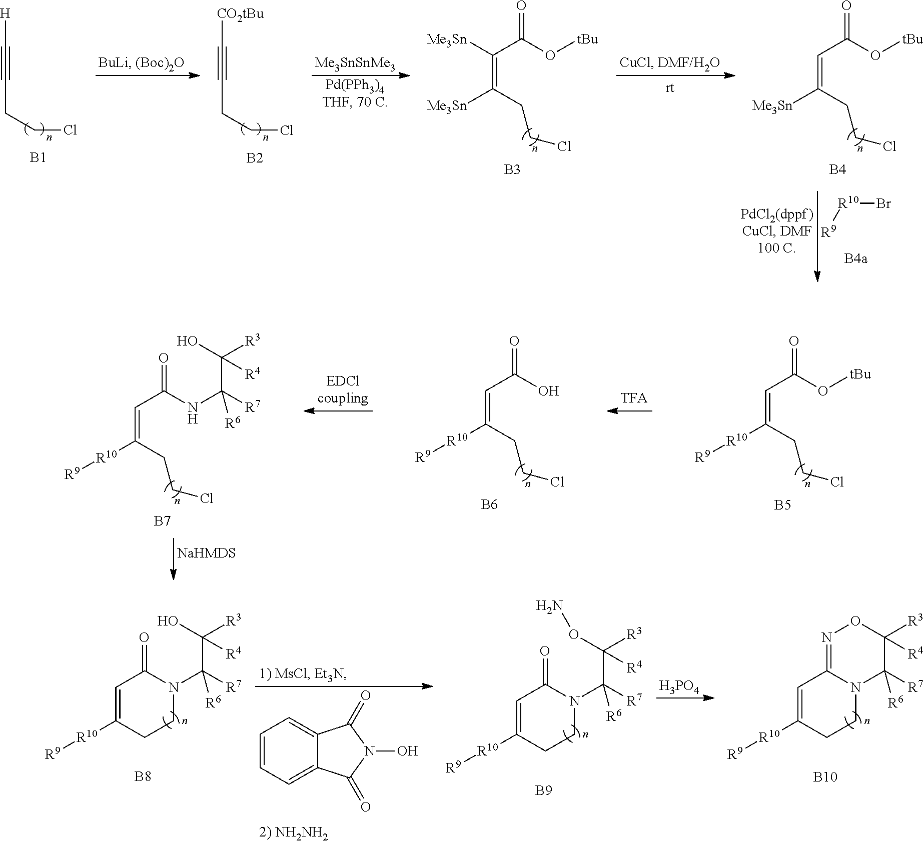

### Example 9

### Step 1

Acetylene B1 (n=2, 1 equiv) was treated with BuLi (1.1 equiv) in anhydrous ether at 0° C. After which, Boc₂O (1.1 equiv) was added and the mixture was allowed to warm up to room temperature. After regular aqueous workup, compound B2 (n=2) was obtained.

### Example 9

### Step 2

Compound B2 (n=2, 1 equiv) was treated with (Me₃Sn)₂ (1 equiv) and Pd(PPh₃)₄ (0.1 equiv) in anhydrous THF. After the mixture was degassed, it was heated at 70° C. for 6 hours. Remove all the volatile then afforded compound B3 (n=2).

### Example 9

### Step 3

Ditin compound B3 (n=2, 3 g) was dissolved in DMF/H₂O (10:1, 80 mL) at room temperature. CuCl (150 mg) was then added. The mixture resulted was stirred at this temperature for 2 hours before Et₂O was added. After regular aqueous workup, the monotin B4 (n=2) was obtained.

### Example 9

### Step 4

Monotin 64 (n=2, 1.8 g, 1 equiv), the bromo compound B4a (R¹⁰=3-MeO-Phenyl, R⁹=4-(4-Methyl-imidazol-1-yl) (1.2 equiv) was mixed with PdCl₂(dppf) (0.1 equiv) and CuCl (0.3 equiv) in DMF. After degas, the mixture was heated at 100° C. for 30 minutes. After regular aqueous work up, compound B5 (n=2, R¹⁰=3-MeO-Phenyl, R⁹=4-(4-Methyl-imidazol-1-yl)) was obtained.

### Example 9

### Step 5

The tbutyl ester 65 (n=2, R¹⁰=3-MeO-Phenyl, R⁹=4-(4-Methyl-imidazol-1-yl)) was dissolved in CH₂Cl₂ and was treated with TFA. After removal of all volatile, the desired acid B6 (n=2, R¹⁰=3-MeO-Phenyl, R⁹=4-(4-Methyl-imidazol-1-yl)) was precipitated out in Et₂O and hexane.

### Example 9

### Step 6

Acid B6 was treated with L-phenylalaminol, EDCI, HOBT, DIEA in DMF overnight. After regular aqueous workup, the desired product B7 (n=2, R¹⁰=3-MeO-Phenyl, R⁹=4-(4-Methyl-imidazol-1-yl), R³═R⁴═R⁶═H, R⁷=Bn) was obtained. MS observed: 468.2.

### Example 9

### Step 7

Amide 67 (n=2, R¹⁰=3-MeO-Phenyl, R⁹=4-(4-Methyl-imidazol-1-yl), R³═R⁴═R⁶═H, R⁷=Bn) (1 equiv) was dissolved in anhydrous THF. The mixture=was cooled down to −30° C. NaHMDS (2.5 equiv) was then added. After the mixture was allowed to warm up to room temperature, the reaction was quenched. And B8 (n=2, R¹⁰=3-MeO-Phenyl, R⁹=4-(4-Methyl-imidazol-1-yl), R³═R⁴═R⁶═H, R⁷=Bn) was then isolated. MS observed: 432.

### Example 9

### Step 8

Lactam B8 is treated with MsCl, Et₃N in THF at −30° C. N-Hydrozylphthalimide is then added. The mixture is then stirred until no starting material left. Remove all the volatile, the residue is then treated with NH₂NH₂ in EtOH. Regular work up will then provide compound B9 ((n=2, R¹⁰=3-MeO-Phenyl, R⁹=4-(4-Methyl-imidazol-1-yl), R³═R⁴═R⁶═H, R⁷=Bn)).

### Example 9

### Step 9

Compound B9 ((n=2, R¹⁰=3-MeO-Phenyl, R⁹=4-(4-Methyl-imidazol-1-yl), R³═R⁴═R⁶═H, R⁷=Bn)) is then treated in BuOH in the presence of 3 equiv of H₃PO₄ overnight at 120° C. Remove all the volatile and purification of the residue will then yield compound B10 ((n=2, R¹⁰=3-MeO-Phenyl, R⁹=4-(4-Methyl-imidazol-1-yl), R³═R⁴═R⁶═H, R⁷=Bn)).

### Assay:

Secretase Reaction and Aβ Analysis in Whole Cells: HEK293 cells overexpressing APP with Swedish and London mutations are treated with the specified compounds for 5 hour at 37° C. in 100 ml of DMEM medium containing 10% fetal bovine serum. At the end of the incubation, total Aβ, Aβ40 and Aβ42 are measured using electrochemiluminescence (ECL) based sandwich immunoassays. Total Aβ is determined using a pair of antibodies TAG-W02 and biotin-4G8, Aβ40 is identified with antibody pairs TAG-G2-10 and biotin-4G8, while Aβ42 is identified with TAG-G2-11 and biotin-4G8. The ECL signal is measured using Sector Imager 2400 (Meso Scale Discovery).

MS Analysis of Aβ Profile: Aβ profile in conditioned media is determined using surface enhanced laser desorption/ionization (SELDI) mass spectrometry. Conditioned media is incubated with antibody W02 coated PS20 ProteinChip array. Mass spectra of Aβ captured on the array is read on SELDI ProteinChip Reader (Bio-Rad) according to manufacture's instructions.

CSF Aβ Analysis: Aβ in rat CSF is determined using MSD technology as described above. Aβ40 is measured using antibody pair Tag-G2-10 and biotin-4G8, while Aβ42 is measured using Tag-anti Aβ42 (Meso Scale Discovery) and biotin-4G8. The ECL signal is measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra are acquired in the linear mode with an acceleration voltage of 20 kV. Each spectrum presented in this work represents an average of 256 laser shots. To prepare the sample-matrix solution, 1 μL of immunoprecipitated Aβ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1-35. (canceled)

36. A compound of the formula (I): 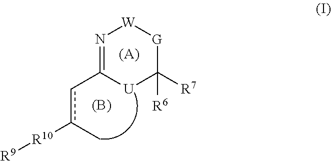
or a pharmaceutically acceptable salt thereof, wherein:
G, U, W, R⁶, R⁷, R⁹, and R¹⁰, are independently selected;
letters (A) and (B) in formula (I) are reference letters to identify the rings present in formula (I);
G is selected from the group consisting of —C(R³)(R⁴)—, —C(O)— and —N(R¹³)—, with the proviso that:
when W is —O— or —S— then G is not —N(R¹³)— or —C(O)—; and
when W is —SO— or —S(O)₂— then G is not —C(O)—;
U is CR⁵ or N;
W is selected from the group consisting of: —O—, —C(O)—, —S—, —S(O)—, —S(O₂)—, and —C(R¹¹)(R¹²)—;
the dotted line in Ring (B) represents an optional bond;
Ring (B) is a 5 to 8 membered ring (including the atoms common to Ring (A)), and: (1) when U is CR⁵ said Ring (B) optionally comprises 1 to 2 heteroatoms independently selected from the group consisting of O, NR² and S, and (2) when U is N said Ring (B) optionally comprises 1 to 2 additional heteroatoms independently selected from the group consisting of O, NR² and S; and said Ring (B) is optionally substituted with 1 to 5 independently selected R²¹ groups;
Each R² is independently selected from the group consisting of: H, —OH, —O-alkyl, —O-(halo substituted alky), —NH(R^{4A}), —N(R^{4A})₂ (wherein each R^{4A} is independently selected), —NH₂, —S(O)R^{4A}, —S(O)(OR^{4A}), —S(O)₂R^{4A}, —S(O)₂(OR^{4A}), —S(O)NHR^{4A}, —S(O)N(R^{4A})₂, —S(O)NH₂, —S(O)₂NHR^{4A}, —S(O)₂N(R^{4A})₂, —S(O)₂NH₂, —CN, —C(O)₂R^{4A}, —C(O)NHR⁴, —C(O)N(R^{4A})₂, —C(O)NH₂, —C(O)R^{4A}, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
each R³ and R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-; wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- group is optionally substituted with 1-5 independently selected R²¹ groups;
each R^{4A} is independently selected from the group consisting of: unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
R⁵ is selected from the group consisting of: H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-; and wherein each of said R⁵ alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- groups are optionally substituted with 1-5 independently selected R²¹ substituents;
R⁶ and R⁷ are each independently selected from the group consisting of: H, —C(O)R¹⁵, —C(O)OR¹⁵, —C(O)N(R¹⁵)(R¹⁶), —C(═NOR¹⁵)R¹⁶, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, benzofusedcycloalkyl, fused benzoheterocycloalkyl, fused heteroarylcycloalkyl, fused heteroarylheterocycloalkyl; and wherein each of said R⁶ and R⁷ alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl-, benzofusedcycloalkyl, fused benzoheterocycloalkyl, fused heteroarylcycloalkyl, and fused heteroarylheterocycloalkyl group is optionally substituted with 1-5 independently selected R²¹ substituents; or
R⁶ and R⁷, taken together with the carbon atom to which they are bound, form a spirocyclic carbocyclic moiety or a spirocyclic heterocyclic moiety, and:
(a) optionally, said spirocyclic carbocyclic moiety is substituted with 1-4 independently selected R²¹ substituents,
(b) optionally, said spirocyclic heterocyclic moiety is substituted with 1-4 independently selected R²¹ substituents,
(c) optionally, said spirocyclic carbocyclic moiety is fused with an aryl, heteroaryl, cycloalkyl, or heterocycloalkyl ring to form a fused ring moiety, and optionally, each ring of said fused ring moiety is substituted with 1-4 independently selected R²¹ substituents;
(d) optionally, said spirocyclic heterocyclic moiety is fused with an aryl, heteroaryl, cycloalkyl, or heterocycloalkyl ring to form a fused ring moiety, and optionally, each ring of said fused ring moiety is substituted with 1-4 independently selected R²¹ substituents;
R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-; wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- group is optionally substituted with 1-3 independently selected R²¹ groups;
R⁹ is selected from the group consisting of: alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, and, optionally, each R⁹ group is substituted with 1-3 independently selected R²¹ groups;
R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl-, 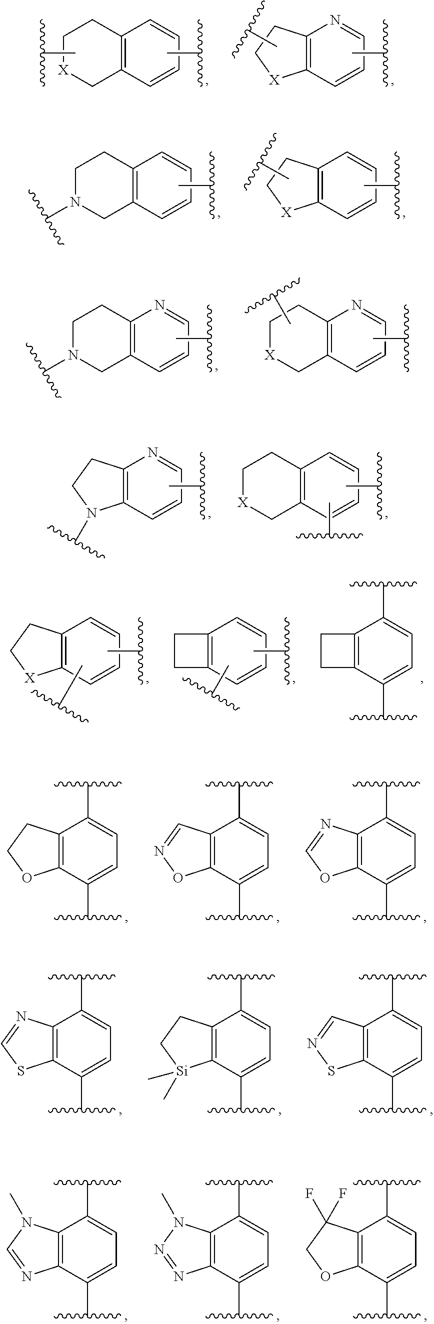 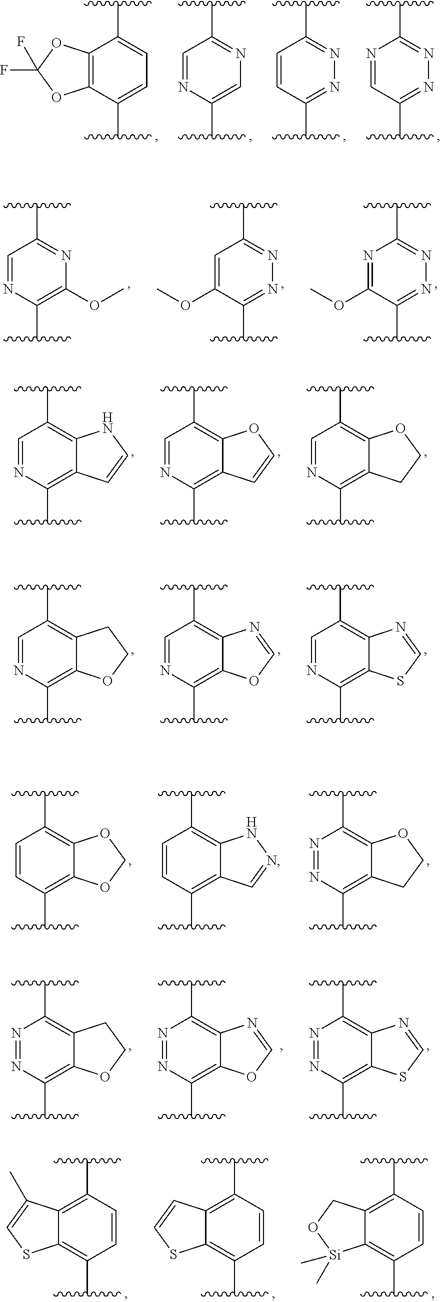 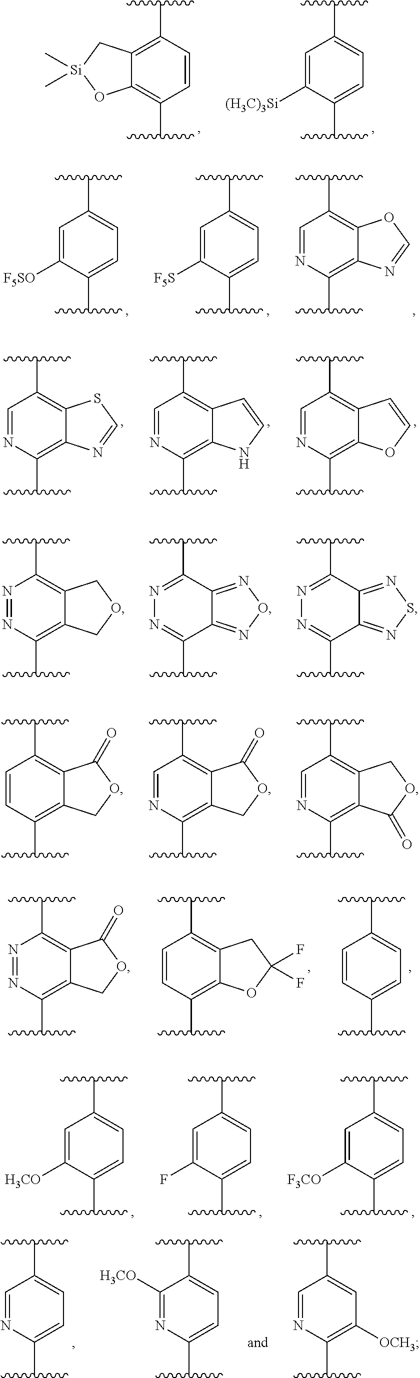
wherein X is selected from the group consisting of: O, N(R¹⁴) or S; and, optionally, each of said R¹⁰ groups are substituted with 1-3 independently selected R²¹ substitutents;
R¹¹ and R¹² are each independently selected from the group consisting of: H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-; and wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- group is optionally substituted with 1-5 independently selected R²¹;
R¹³ is independently selected from the group consisting of H, alkyl, arylalkyl, heteroarylalkyl-, cycloalkylalkyl-, heterocycloalkylalkyl-, arylcycloalkylalkyl-, heteroarylcycloalkylalkyl-, arylheterocycloalkylalkyl-, heteroarylheterocycloalkylalkyl-, cycloalkyl, arylcycloalkyl-, heteroarylcycloalkyl-, heterocycloalkyl-, arylheterocycloalkyl-, heteroarylheterocycloalkyl-, alkenyl, arylalkenyl-, cycloalkenyl, arylcycloalkenyl-, heteroarylcycloalkenyl-, heterocycloalkenyl, arylheterocycloalkenyl-, heteroarylheterocycloalkenyl-, alkynyl, arylalkynyl-, aryl, cycloalkylaryl-, heterocycloalkylaryl-, heterocycloalkenylaryl-, heteroaryl, cycloalkylheteroaryl-, heterocycloalkylheteroaryl-, cycloalkenylaryl-, heterocycloalkenylaryl-, —OR¹⁵, —CN, —C(O)R⁸, —C(O)OR⁸, —S(O)R¹⁰, —S(O)₂R¹⁰, —C(O)N(R¹¹)(R¹²), —S(O)N(R¹¹)(R¹²), —S(O)₂N(R¹¹)(R¹²), —NO₂, —N═C(R⁸)₂ and —N(R⁸)₂; and wherein said R¹³ alkyl, arylalkyl-, heteroarylalkyl-, cycloalkylalkyl-, heterocycloalkylalkyl-, arylcycloalkylalkyl-, heteroarylcycloalkylalkyl-, arylheterocycloalkylalkyl-, heteroarylheterocycloalkylalkyl-, cycloalkyl, arylcycloalkyl-, heteroarylcycloalkyl-, heterocycloalkyl, arylheterocycloalkyl-, heteroarylheterocycloalkyl-, alkenyl, arylalkenyl-, cycloalkenyl, arylcycloalkenyl-, heteroarylcycloalkenyl-, heterocycloalkenyl, arylheterocycloalkenyl-, heteroarylheterocycloalkenyl-, alkynyl, arylalkynyl-, aryl, cycloalkylaryl-, heterocycloalkylaryl-, heterocycloalkenylaryl-, heteroaryl, cycloalkylheteroaryl-, heterocycloalkylheteroaryl-, cycloalkenylaryl-, and heterocycloalkenylaryl- groups are optionally substituted with 1 to 5 groups independently selected from the group consisting of: alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁸)(R¹⁷), —CH₂—R¹⁵; —CH₂N(R¹⁵)(R¹⁶), —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, ═NOR¹⁵, —N₃, —NO₂ and —S(O)₂R^{15A},
R¹⁴ is selected from the group consisting of H, alkyl, alkenyl, alkynyl, cycloalkyl, cycloalkylalkyl-, cycloalkenyl, heterocyclyl, heterocyclenyl, heterocyclylalkyl-, heterocyclyalkenyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), and wherein each alkyl, alkenyl, alkynyl, cycloalkyl, cycloalkylalkyl-, cycloalkenyl, heterocyclyl, heterocyclenyl, heterocyclylalkyl-, heterocyclyalkenyl-, aryl, arylalkyl-, heteroaryl, heteroarylalkyl- group is optionally substituted with 1-5 independently selected R²¹ groups;
Each R^{15A} is independently selected from the group consisting of alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)₁₋₅-alkyl, (R¹⁸)₁₋₅-cycloalkyl, (R¹⁸)₁₋₅-cycloalkylalkyl-, (R¹⁸)₁₋₅-heterocyclyl, (R¹⁸)₁₋₅-heterocyclylalkyl-, (R¹⁸)₁₋₅-aryl, (R¹⁸)₁₋₅-arylalkyl-, (R¹⁸)₁₋₅-heteroaryl and (R¹⁸)₁₋₅-heteroarylalkyl-; and wherein each R¹⁸ in each group can be on any substitutable atom;
Each R^{16A} is independently selected from the group consisting of alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)₁₋₅-alkyl, (R¹⁸)₁₋₅-cycloalkyl, (R¹⁸)₁₋₅-cycloalkylalkyl-, (R¹⁸)₁₋₅-heterocyclyl, (R¹⁸)₁₋₅-heterocyclylalkyl-, (R¹⁸)₁₋₅-aryl, (R¹⁸)₁₋₅-arylalkyl-, (R¹⁸)₁₋₅-heteroaryl and (R¹⁸)₁₋₅-heteroarylalkyl-; and wherein each R¹⁸ in each group can be on any substitutable atom;
R¹⁵, R¹⁶ and R¹⁷ are independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)₁₋₅-alkyl, (R¹⁸)₁₋₅-cycloalkyl, (R¹⁸)₁₋₅-cycloalkylalkyl-, (R¹⁸)₁₋₅-heterocyclyl, (R¹⁸)₁₋₅-heterocyclylalkyl-, (R¹⁸)₁₋₅-aryl, (R¹⁸)₁₋₅-arylalkyl-, (R¹⁸)-heteroaryl and (R¹⁸)₁₋₅-heteroarylalkyl-; and wherein each R¹⁸ in each group can be on any substitutable atom;
Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NH R¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
or, two R¹⁸ moieties on adjacent carbons can be taken together with the atoms to which they are bound to form: 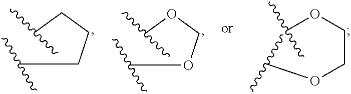
R¹⁹ is selected from the group consisting of: alkyl, cycloalkyl, aryl, arylalkyl and heteroarylalkyl;
R²⁰ is selected from the group consisting of: alkyl, cycloalkyl, aryl, halo substituted aryl, arylalkyl, heteroaryl and heteroarylalkyl;
Each R²¹ is independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SF_{S}, —OSF₅, —Si(R^{15A})₃ wherein each R^{15A} is independently selected —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)²R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, —N₃, —NO₂ and —S(O)₂R^{15A}; and, optionally, each of said alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl R²¹ groups are substituted with 1 to 5 independently selected R²² groups; and
Each R²² is independently selected from the group consisting of: alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R^{15A})₃ wherein each R¹⁵ is independently selected —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R^{16A}, —N(R¹⁵)S(O)₂R^{16A}—CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)_{C}(O)N(R¹⁶)(R¹⁷), —CH₂—N(R¹⁵)_{C}(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R^{15A} and —S(O)₂R^{15A}.

37. The compound of claim 36 wherein said R¹⁹ is phenyl substituted with one R²¹ group, and said R⁹ is imidazolyl substituted with one R²¹ group, wherein each R²¹ is independently selected.

38. The compound of claim 36 wherein the R⁹—R¹⁰— moiety is selected from the group consisting of:

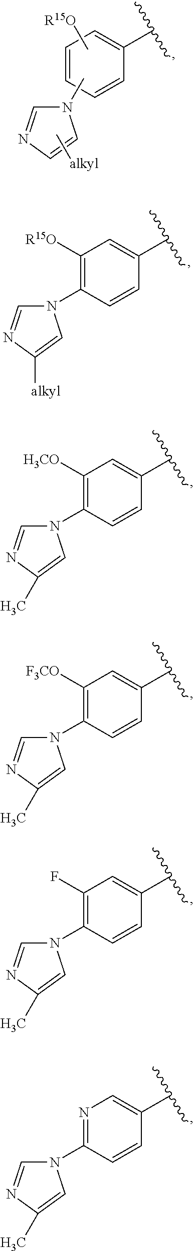

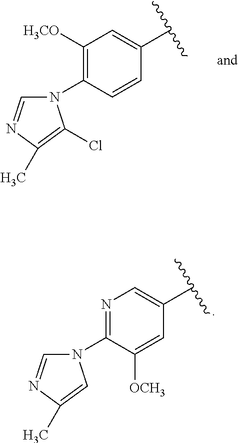

39. The compound of claim 36 wherein R⁶ is alkyl, and R⁷ is a substituted aryl group.

40. The compound of claim 36 wherein R⁷ is:
phenyl, or
phenyl substituted with one or more independently selected R²¹ groups, or
phenyl substituted with 1 to 3 independently selected R²¹ groups, or
phenyl substituted with 1 to 3 R²¹ groups, and each R²¹ group is the same or different halo, or
phenyl substituted with 1 to 3 F, or
phenyl substituted with one —CN group, or
phenyl substituted with one or two —CF₃ groups, or
phenyl substituted with R²¹ groups and at least one R²¹ group selected from the group consisting of: —SF₅, —OSF₅ and —Si(R^{15A})₃, wherein each R^{15A} is independently selected, or
phenyl substituted with R²¹ groups and at least one R²¹ group selected from the group consisting of: —SF₅, —OSF₅ and —Si(R^{15A})₃, and each R^{15A} is the same or different alkyl group, or
phenyl substituted with R²¹ groups and at least one R²¹ group selected from the group consisting of: —SF₅, —OSF₅ and —Si(R^{15A})₃, and each R^{15A} is the same or different alkyl group, or
phenyl substituted with R²¹ groups and at least one R²¹ group selected from the group consisting of: —SF₅, —OSF₅ and —Si(CH₃)₃.

41. The compound of claim 36 wherein R⁷ is selected from the group consisting of:

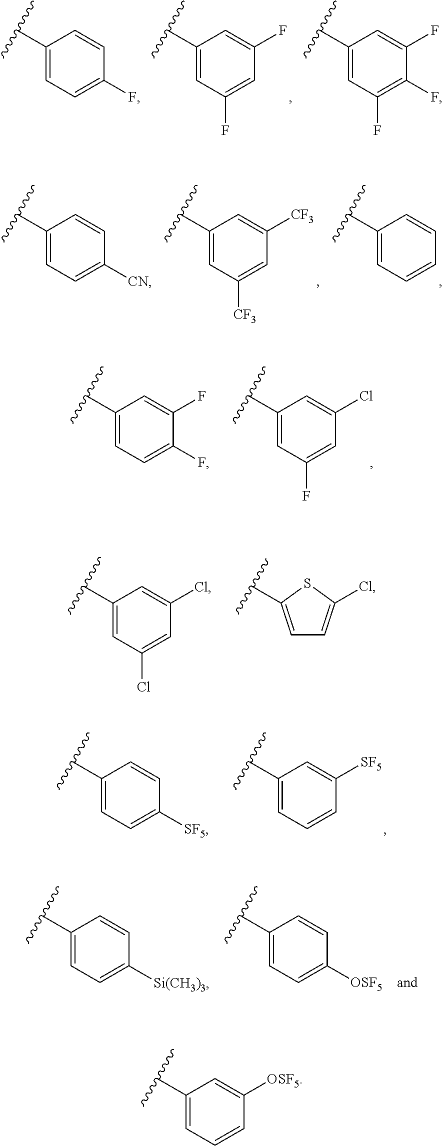

42. The compound of claim 36 wherein:
(1) W is —O—, G is C(R³)(R⁴), and U is CR⁵,
(2) W is —O—, G is C(R³)(R⁴), and U is N,
(3) W is —S—, G is C(R³)(R⁴), and U is CR⁵,
(4) W is —S—, G is C(R³)(R⁴), and U is N,
(5) W is —C(O)—, G is C(R³)(R⁴), and U is CR⁵,
(6) W is —C(O)—, G is C(R³)(R⁴), and U is N,
(7) W is —C(O)—, G is C(O)—, and U is CR⁵,
(8) W is —C(O)—, G is C(O)—, and U is N,
(9) W is —C(O)—, G is N(R¹³), and U is CR⁵,
(10) W is —C(O)—, G is N(R¹³), and U is N,
(11) W is —S(O)—, G is C(R³)(R⁴), and U is CR⁵,
(12) W is —S(O)—, G is C(R³)(R⁴), and U is N,
(13) W is —S(O)—, G is N(R¹³), and U is CR⁵,
(14) W is —S(O)—, G is N(R¹³), and U is N,
(15) W is —S(O₂)—, G is C(R³)(R⁴), and U is CR⁵,
(16) W is —S(O₂)—, G is C(R³)(R⁴), and U is N,
(17) W is —S(O₂)—, G is N(R¹³), and U is CR⁵,
(18) W is —S(O₂)—, G is N(R¹³), and U is N,
(19) W is —C(R¹¹)(R¹²)—, G is C(R³)(R⁴), and U is CR⁵,
(20) W is —C(R¹¹)(R¹²)—, G is C(R³)(R⁴), and U is N,
(21) W is —C(R¹¹)(R¹²)—, G is C(O)—, and U is CR⁵,
(22) W is —C(R¹¹)(R¹²)—, G is C(O)—, and U is N,
(23) W is —C(R¹¹)(R¹²)—, G is N(R¹³), and U is CR⁵, or
(24) W is —C(R¹¹)(R¹²)—, G is N(R¹³), and U is N.

43. The compound of claim 36 wherein: (a) the R⁹—R¹⁰— moiety is selected from the group consisting of: 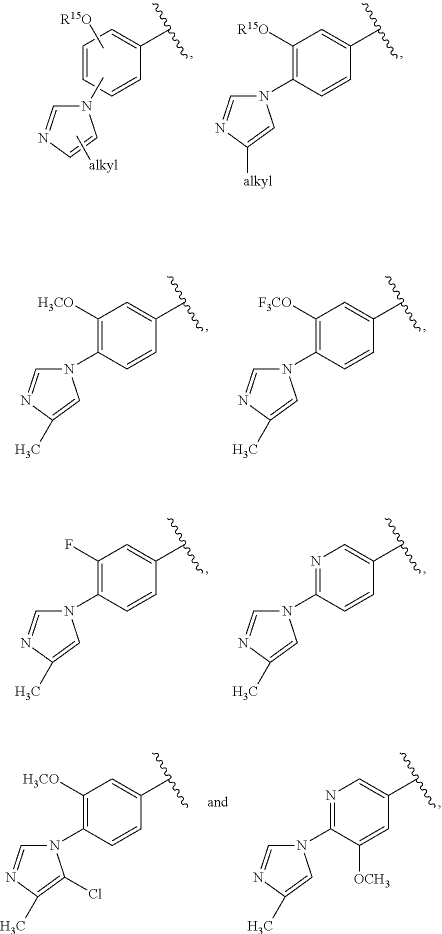
(b) R⁶ is alkyl, (c) R⁷ is a substituted aryl group, and wherein W, G and U are selected from the group consisting of:
(1) W is —O—, G is C(R³)(R⁴), and U is CR⁵,
(2) W is —O—, G is C(R³)(R⁴), and U is N,
(3) W is G is C(R³)(R⁴), and U is CR⁵,
(4) W is —S—, G is C(R³)(R⁴), and U is N,
(5) W is —C(O)—, G is C(R³)(R⁴), and U is CR⁵,
(6) W is —C(O)—, G is C(R³)(R⁴), and U is N,
(7) W is —C(O)—, G is C(O)—, and U is CR⁵,
(8) W is —C(O)—, G is C(O)—, and U is N,
(9) W is —C(O)—, G is N(R¹³), and U is CR⁵,
(10) W is —C(O)—, G is N(R¹³), and U is N,
(11) W is —S(O)—, G is C(R³)(R⁴), and U is CR⁵,
(12) W is —S(O)—, G is C(R³)(R⁴), and U is N,
(13) W is —S(O)—, G is N(R¹³), and U is CR⁵,
(14) W is —S(O)—, G is N(R¹³), and U is N,
(15) W is —S(O₂)—, G is C(R³)(R⁴), and U is CR⁵,
(16) W is —S(O₂)—, G is C(R³)(R⁴), and U is N,
(17) W is —S(O₂)—, G is N(R¹³), and U is CR⁵,
(18) W is —S(O₂)—, G is N(R¹³), and U is N,
(19) W is —C(R¹¹)(R¹²)—, G is C(R³)(R⁴), and U is CR⁵,
(20) W is —C(R¹¹)(R¹²)—, G is C(R³)(R⁴), and U is N,
(21) W is —C(R¹¹)(R¹²)—, G is C(O)—, and U is CR⁵,
(22) W is —C(R¹¹)(R¹²)—, G is C(O)—, and U is N,
(23) W is —C(R¹¹)(R¹²)—, G is N(R¹³), and U is CR⁵, and
(24) W is —C(R¹¹)(R¹²)—, G is N(R¹³), and U is N.

44. The compound of claim 43 wherein R⁷ is:
phenyl, or
phenyl substituted with one or more independently selected R²¹ groups, or
phenyl substituted with 1 to 3 independently selected R²¹ groups, or
phenyl substituted with 1 to 3 R²¹ groups, and each R²¹ group is the same or different halo, or
phenyl substituted with 1 to 3 F, or
phenyl substituted with one —CN group, or
phenyl substituted with one or two —CF₃ groups, or
phenyl substituted with R²¹ groups and at least one R²¹ group selected from the group consisting of: —SF₅, —OSF₅ and —Si(R^{15A})₃, wherein each R^{15A} is independently selected, or
phenyl substituted with R²¹ groups and at least one R²¹ group selected from the group consisting of: —SF₅, —OSF₅ and —Si(R^{15A})₃, and each R^{15A} is the same or different alkyl group, or
phenyl substituted with R²¹ groups and at least one R²¹ group selected from the group consisting of: —SF₅, —OSF₅ and —Si(R^{15A})₃, and each R^{15A} is the same or different alkyl group, or
phenyl substituted with R²¹ groups and at least one R²¹ group selected from the group consisting of: —SF₅, —OSF₅ and —Si(CH₃)₃.

45. The compound of claim 44 wherein R⁷ is selected from the group consisting of:

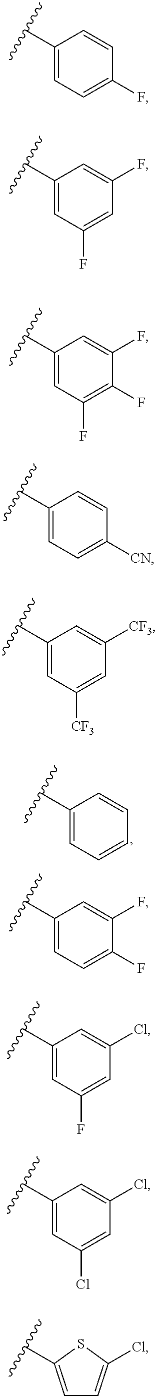

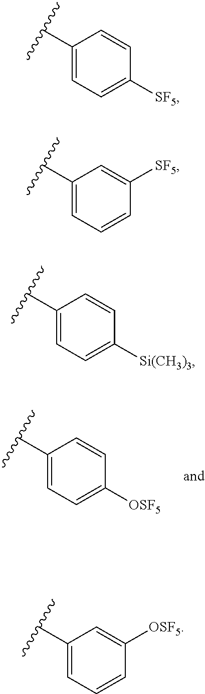

46. The compound of claim 36 wherein the compounds of formula (I) are selected from the group consisting of: 1A, 2A, 3A, 4A, 5A, 6A, 7A, 8A, 9A, 10A, 1B, 2B, 3B, 4B, 5B, 6B, 7B, 8B, 9B, 10B, 1C, 2C, 3C, 4C, 5C, 6C, 7C, 8C, 9C, 10C, 1D, 2D, 3D, 4D, 5D, 6D, 7D, 8D, 9D, 10D, 1E, 2E, 3E, 1F, 2F, 3F, 4F, 1G, 2G, 3G, and 4G.

47. The compound of claim 36 wherein:
R⁶ is alkyl;
R⁷ is:
phenyl, or
phenyl substituted with one or more independently selected R²¹ groups, or
phenyl substituted with 1 to 3 independently selected R²¹ groups, or
phenyl substituted with 1 to 3 R²¹ groups, and each R²¹ group is the same or different halo, or
phenyl substituted with 1 to 3 F, or
phenyl substituted with one —CN group, or
phenyl substituted with one or two —CF₃ groups, or
phenyl substituted with R²¹ groups and at least one R²¹ group selected from the group consisting of: —SF₅, —OSF₅ and —Si(R^{15A})₃, wherein each R^{15A} is independently selected, or
phenyl substituted with R²¹ groups and at least one R²¹ group selected from the group consisting of: —SF₅, —OSF₅ and —Si(R^{15A})₃, and each R^{15A} is the same or different alkyl group, or
phenyl substituted with R²¹ groups and at least one R²¹ group selected from the group consisting of: —SF₅, —OSF₅ and —Si(R^{15A})₃, and each R^{15A} is the same or different alkyl group, or
phenyl substituted with R²¹ groups and at least one R²¹ group selected from the group consisting of: —SF₅, —OSF₅ and —Si(CH₃)₃; and
the R⁹— R¹⁰— moiety is selected from the group consisting of:

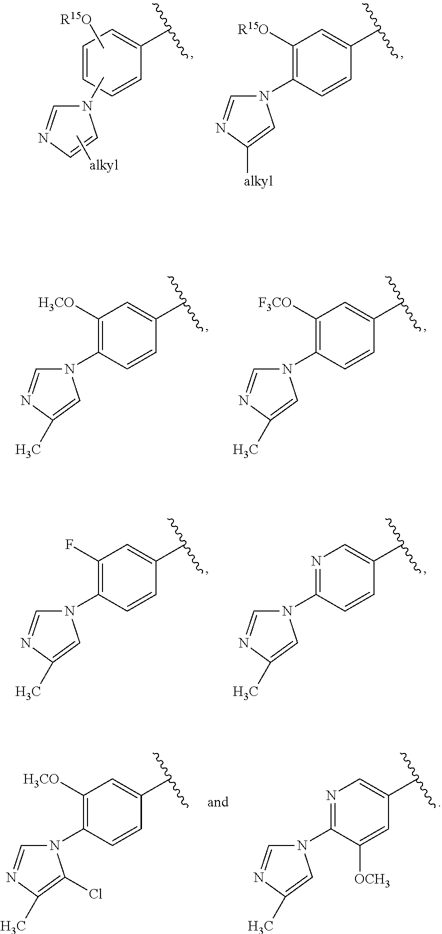

48. The compound of claim 47 wherein R⁷ is selected from the group consisting of:

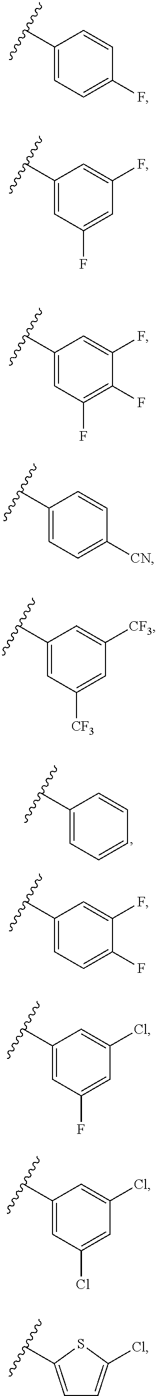

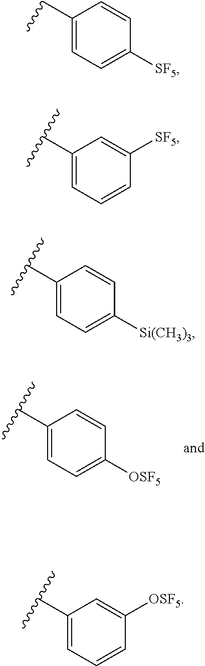

49. The compound of claim 36 selected from the group consisting of the final compounds of Examples 1, 2, and 4 to 9, and 1H to 11H.

50. A pharmaceutical composition comprising a therapeutically effective amount of a compound of claim 36 or a pharmaceutically acceptable salt thereof, and a pharmaceutically acceptable carrier.

51. A method of modulating gamma-secretase comprising administering an effective amount of one or more compounds of claim 36 or a pharmaceutically acceptable salt thereof to a patient in need of such treatment.

52. A method of inhibiting the deposition of amyloid protein in, on or around neurological tissue, comprising administering an effective amount of one or more compounds of claim 36 or a pharmaceutically acceptable salt thereof to a patient in need of treatment.
